(19) United States
(12) Patent Application Publication
(10) Pub. No.: US 2025/0250638 A1
(43) Pub. Date: Aug. 7, 2025

(54) GENOMIC AND METHYLATION BIOMARKERS FOR PREDICTION OF COPY NUMBER LOSS / GENE DELETION

(71) Applicant: GUARDANT HEALTH, INC., Palo Alto, CA (US)

(72) Inventors: Sean GORDON, San Diego, CA (US); Catalin BARBACIORU, Fremont, CA (US); Evan WARNER, vancouver (CA); Andrew M. GROSS, Cincinnati, OH (US); Denis TOLKUNOV, Dublin, CA (US); Geethika YALAMANCHILI, Palo Alto, CA (US)

(21) Appl. No.: 18/907,170

(22) Filed: Oct. 4, 2024

Related U.S. Application Data
(60) Provisional application No. 63/559,749, filed on Feb. 29, 2024.
(60) Provisional application No. 63/588,630, filed on Oct. 6, 2023.

(51) Int. Cl. C12Q 1/6886; C12Q 1/48

(52) U.S. Cl. CPC C12Q 1/6886; C12Q 1/48; C12Q 2600/106; C12Q 2600/154

(57) ABSTRACT

Disclosed herein are methods, compositions, and devices for use in diagnosis and treatment of cancer. The methods include sequencing a panel of regions in cell-free nucleic acid molecules and detecting one or more biomarkers that are indicative of a cancer.

### CROSS-REFERENCE TO RELATED APPLICATION

The present application claims the benefit of U.S. Provisional Application No. 63/559,749, filed Feb. 29, 2024 and U.S. Provisional Application No. 63/588,630, filed Oct. 6, 2023. Both are incorporated by reference in their entirety for all purposes.

### BACKGROUND

Cancer is a major cause of disease worldwide. Each year, tens of millions of people are diagnosed with cancer around the world, and more than half of the patients eventually die from it. In many countries, cancer ranks the second most common cause of death following cardiovascular diseases. Early detection is associated with improved outcomes for many cancers.

The 5′-methylthioadenosine phosphorylase (MTAP) gene is located in the chromosomal region 9p21. MTAP deletion is a frequent event in a wide variety of human cancers; however, its biological role in tumorigenesis remains unclear. The MTAP gene is flanked by CDKN2A and miR-31 and the gene is frequently co-deleted, in many different tumors, with the CDKN2A and CDKN2B genes that encode the tumor suppressors p15, p16, p19 and with the genes for interferons alpha and beta that lie telomeric to miR-31, Selective MTAP deficiency, without co-deletion of the CDKN2 genes, has also been reported, due either to selective deletion of the MTAP locus or to methylation of the MTAP promoter.

### SUMMARY OF INVENTION

Described herein is a method, including: detecting methylation in at least one of a plurality of sites; generating a plurality of one or more metrics for each of the plurality of sites; and processing the one or more metrics to characterize at least one cis regulatory network element in a regulatory network. In various embodiment, characterizing the at least one cis regulatory network element includes determine the presence, absence of at least one cis regulatory network element in a regulatory network. In various embodiment, the at least one cis regulatory network element includes, for example, a genetic variant, an indel, a homodel, a fusion, and/or other mutation. In various embodiments, the determination characterizes a sample. In other embodiments, the at least one cis regulatory network element is capable of altering a regulatory sub-circuit. In other embodiments, the cis regulatory network element includes an epigenetic regulator. In other embodiments, the regulatory sub-circuit is bi-directional. In other embodiments, the regulatory sub-circuit increases methylation in plurality of targets. In other embodiments, the regulatory sub-circuit increases methylation in plurality of targets. In other embodiments, the regulatory network includes an additional cis regulatory network element. In other embodiments, the additional cis regulatory network element is capable of altering the regulatory sub-circuit. In other embodiments, the additional cis regulatory network element includes a regulatory node. In other embodiments, the regulatory node is a point of interdiction. In other embodiments, the regulatory sub-circuit includes an additional regulatory node. In other embodiments, the additional regulatory node is a point of interdiction.

Described herein is a method, including: generating a methylation profile for at least one nucleic acid sequence obtained from a human subject; and selecting a treatment suitable for the human subject based on the methylation profile. In other embodiments, the methylation profile includes at least one differentially methylated region (DMR) In other embodiments, the at least one DMR is determined based on a comparison to a threshold determined from one or more healthy subjects. In other embodiments, the methylation profile characterizes a gain of function for at least one or more genes. In other embodiments, the methylation profile characterizes a loss of function for at least one or more genes. In other embodiments, the methylation profile characterizes a genomic mutation. In other embodiments, the methylation profile characterizes a gene target. In other embodiments, the methylation profile characterizes gene dysfunction. In other embodiments, the characterization is of a gene target capable of conferring synthetic lethality. In other embodiments, the characterization is based on hypermethylation status. In other embodiments, the one or more genes includes CDKN2A. In other embodiments, the one or more genes includes a metabolic enzyme. In other embodiments, the metabolic enzyme includes Methylthioadenosine Phosphorylase (MTAP). In other embodiments, the methylation profile is detected using a methyl binding domain (MBD) partitioning assay. In other embodiments, the MBD partitioning assay includes combining a plurality of nucleic acid molecules derived from the human subject with a solution including an amount of methyl binding domain (MBD) proteins to produce a nucleic acid-MBD protein solution; and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least the threshold cytosine-guanine content. In other embodiments, the treatment includes an PRMT5-MTA5 inhibitor and/or immunotherapy. In other embodiments, the genomic mutation and/or gene dysfunction modulates PRMT5-MTA complex and/or MTA accumulation. In other embodiments, the e detection includes a method selected from the group consisting of Next Generation Sequencing (NGS), Third-Generation Sequencing (TGS), Sanger Sequencing, Microarray Analysis, PCR, Fluorescence In Situ Hybridization (FISH), Real-Time PCR (RT-PCR), Digital PCR (dPCR), Droplet Digital PCR (ddPCR), Targeted Sequencing, Nanopore Sequencing, Short-Read Sequencing, Long-Read Sequencing Technologies (e.g., PacBio and Nanopore). In other embodiments, the e patient is afflicted with a cancer selected form the group consisting of: breast cancer, lung cancer, prostate cancer, colorectal cancer, skin cancer, ovarian cancer, pancreatic cancer, leukemia, lymphoma, liver cancer, cervical cancer, brain cancer, stomach cancer, kidney cancer, bladder cancer, thyroid cancer, esophageal cancer, melanoma, testicular cancer, epithelioid sarcoma, relapsed or refractory follicular lymphoma, non-Hodgkin lymphomas (NHL), relapsed or refractory adult T-cell leukaemia/lymphoma (R/R ATL), follicular lymphoma, castration-resistant prostate cancer (CRPC), small cell lung cancer, and sarcoma. In other embodiments, the sample is selected from the group consisting of blood, plasma, cell free DNA(cfDNA), cell free RNA(cfRNA), saliva, urine, cerebrospinal fluid, synovial fluid, amniotic fluid, lymph, semen, vaginal fluid, tears, breast milk, mucus, sweat, pericardial fluid, pleural fluid, peritoneal fluid, bile, and interstitial fluid. In other embodiments, the method includes selecting a treatment suitable for the human subject includes use of a database. In other embodiments, the database includes a plurality of nucleic acid sequence information, including methylation status from a plurality of subjects. In other embodiments, selecting a treatment includes identifying from a plurality of subjects with matching genetic, epigenetic information, prior treatment of the plurality of subjects with matching genetic information.

Described herein is a method, including: generating a methylation profile using a methyl binding domain (MBD) partitioning assay for at least one nucleic acid sequence obtained from a human subject; selecting a treatment suitable for the human subject based on the methylation profile using a database, wherein the database includes a plurality of nucleic acid sequence information, and methylation status from a plurality of subjects and identifying from the plurality of subjects with matching genetic and epigenetic information, prior treatment of the plurality of subjects with matching genetic information.

Described herein is a method, including: determining a state of biological molecules obtained from a sample derived from a human subject, and detecting biological molecules in the sample. In other embodiments, the biological molecules are one or more of: DNA, methylated DNA, RNA, methylated RNA, proteins, and peptides. In other embodiments, the method includes testing combining a plurality of nucleic acid molecules derived from a subject with a solution including an amount of methyl binding domain (MBD) proteins to produce a nucleic acid-MBD protein solution; and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least the threshold cytosine-guanine content. In other embodiments, the wash of the plurality of washes is performed with a solution having a concentration of sodium chloride (NaCl) and produces a nucleic acid fraction of the number of nucleic acid fractions having a range of binding strengths to MBD proteins. In other embodiments, the method includes determining that a first nucleic acid fraction is associated with a first partition of a plurality of partitions of nucleic acids, the first partition corresponding to a first range of binding strengths to MBD proteins, attaching a first molecular barcode to nucleic acids of the first nucleic acid fraction, the first molecular barcode being included in a first set of molecular barcodes associated with the first partition, determining that a second nucleic acid fraction is associated with a second partition of the plurality of partitions of nucleic acids, the second partition corresponding to a second range of binding energies to MBD proteins different from the first range of binding strengths to MBD proteins, and attaching a second molecular barcode to nucleic acids of the second nucleic acid fraction, the second molecular barcode being included in a second set of molecular barcodes associated with the second partition. In other embodiments, the method includes combining at least a portion of the number of nucleic acid fractions with an amount of restriction enzyme that cleaves molecules with one or more unmethylated cytosines to produce at least a portion of the plurality of samples used to produce the sequencing reads, wherein the threshold amount of methylated cytosines corresponds to a minimum frequency of methylated cytosines within a region having at least the threshold cytosine-guanine content.

In other embodiments, the method includes combining at least a portion of the number of nucleic acid fractions with an amount of a restriction enzyme that cleaves molecules with one or more methylated cytosines to produce at least a portion of the plurality of samples used to produce the sequencing reads, wherein the threshold amount of unmethylated cytosines corresponds to a maximum frequency of methylated cytosines that are not cleaved within a region having at least the threshold cytosine-guanine content. In other embodiments, the method includes testing including sequencing nucleic acid molecules derived from a sample obtained from a subject, analyzing sequence reads derived from the sequencing to identify one or more driver mutations in the nucleic acid molecules, and using information about the presence, absence, or amount of the one or more driver mutations in the nucleic acid molecules to identify a tumor in the subject. In other embodiments, the nucleic acid molecules are cell-free DNA. In other embodiments, the sample is at least one of blood, serum, plasma or tissue. In other embodiments, the method includes determination of treatment for the subject. In other embodiments, a limit of detection for the model to determine tumor fraction of samples is no greater than 0.05%. In other embodiments, the one or more driver mutations includes a somatic variant detected at a mutant allele frequency (MAF) of no more than 0.05%. In other embodiments, the one or more driver mutations includes a fusion detected at a mutant allele frequency (MAF) of no more than 0.1%. In other embodiments, the method includes detecting mutation distributions for each of one or more driver mutations, wherein the mutation distribution for each of the one or more driver mutations is detected with a correlation of at least 0.99 to a mutation distribution of the driver mutation detected in a cohort of the subject by tissue genotyping. In other embodiments, the method detects the tumor in the subject with a sensitivity of at least 85%, a specificity of at least 99%, and a diagnostic accuracy of at least 99%. In other embodiments, the method includes identifying circulating tumor DNA (ctDNA) and one or more driver mutations in the ctDNA. In other embodiments, the method includes obtaining, by a computing system having one or more hardware processors and memory, testing sequence data from a subject, the testing sequence data including testing sequencing reads derived from a sample of the subject, analyzing, by the computing system, the testing sequencing reads to determine a first quantitative measure derived from the testing sequencing reads to genomic regions of a reference genome, analyzing, by the computing system, the testing sequencing reads to determine a second quantitative measure derived from the testing sequencing reads to genomic regions of a reference genome, determining, by the computing system, a metric based on the first quantitative and the second quantitative measure, and generating, by the computing system, an input vector that includes the metrics, determining, by the computing system, an indication of cancer status in the subject by providing the input vector to a model that implements one or more machine learning techniques to generate indications of cancer status in subjects, the model including weights for individual classification regions of a plurality of classification regions and at least a portion of the weights of the individual classification regions being different from one another. In other embodiments, the individual testing sequencing reads include a nucleotide sequence corresponding to a fragment of a nucleic acid included in the sample and the individual testing sequencing reads correspond to molecules having a threshold amount of methylated cytosines included in regions of the nucleotide sequence having at least the threshold cytosine-guanine content, the first quantitative measure derived from the testing sequencing reads that correspond to individual classification regions of a plurality of classification regions at least a portion of the individual classification regions of the plurality of classification regions corresponding to genomic regions of a reference genome that have the threshold amount of methylated cytosines in subjects in which cancer is detected and that have at least the threshold cytosine-guanine content, the second quantitative measure derived from the testing sequencing reads that correspond to individual control regions a plurality of control regions, individual control regions of the plurality of control regions corresponding to additional genomic regions of the reference genome that have at least the threshold cytosine-guanine content and that have at least the threshold amount of methylated cytosines in subjects in which cancer is detected and in additional subjects in which cancer is not detected. In other embodiments, the method includes obtaining, by the computing system having one or more hardware processors and memory, training sequence data including training sequencing reads derived from a plurality of samples of a plurality of training subjects, individual training sequencing reads including a nucleotide sequence corresponding to a fragment of a nucleic acid included in a sample of the plurality of samples and individual training sequencing reads corresponding to molecules having a threshold amount of methylated cytosines included in regions of the nucleotide sequence having at least a threshold cytosine-guanine content, analyzing, by the computing system, the training sequencing reads to determine an additional first quantitative measure derived from the training sequencing reads that corresponds to individual classification regions of the plurality of classification regions, analyzing, by the computing system, the training sequencing reads to determine an additional second quantitative measure derived from the training sequencing reads that correspond to a plurality of control regions, determining, by the computing system, an additional metric for the individual classification regions of the plurality of classification regions based on the additional first quantitative measure for the individual classification regions and the additional second quantitative measure for the plurality of control regions, generating, by the computing device, training data that includes the additional metric for the individual classification regions of the plurality of classification regions for the training sequence reads from samples of the plurality of training subjects, implementing, by the computing system and using the training data, one or more machine learning algorithms to generate the model to determine the indications of cancer status in subjects based on amounts of methylated cytosines in at least a portion of the plurality of classification regions. In other embodiments, the one or more machine learning algorithms include one or more classification algorithms. In other embodiments, the one or more machine learning algorithms include one or more regression algorithms, and the indication corresponds to an estimate of tumor fraction of the sample. In other embodiments, the training sequencing reads comprise a first portion of the training sequence data and additional training sequencing reads comprise a second portion of the training sequence data, wherein the additional training sequencing reads are different from the training sequencing reads; and the method including analyzing, by the computing system, at least one of the first portion of the training sequence data or the second portion of the training sequence data to determine an individual frequency of a plurality of variants present in an individual sample of the plurality of samples, determining, by the computing system and for the individual sample, a variant of the plurality of variants having a maximum frequency that corresponds to the individual frequency having a greatest value among individual frequencies derived from an individual sample, and determining, by the computing system, individual measures of tumor fraction for an individual sample based on the greatest value of the individual frequencies derived from the individual sample. In other embodiments, the training data includes the individual measures of tumor fraction for the individual samples of the plurality of samples, and the model is generated based on the individual measures of tumor fraction for the individual samples of the plurality of samples. In other embodiments, the method includes generating, by a computing system including processing circuitry and memory, a data file including first tokens generated using a first hash function, individual first tokens corresponding to a respective individual of a group of individuals having data stored by a molecular data repository, sending, by the computing system, the data file to a electronic medical record, including for example, patient information, health insurance claims, lifestyle data, management system, obtaining, by the computing system and from the electronic medical record, including for example, patient information, health insurance claims, lifestyle data, management system, in response to the data file, health data corresponding to the group of individuals, generating, by the computing system, a number of identifiers using a second hash function that is different from the first hash function, each identifier corresponds to one or more tokens related to each individual of the group of individuals, obtaining, by the computing system and using the number of identifiers, second data from the molecular data repository for the group of individuals, determining, by the computing system, respective portions of the first data that correspond to respective portions of the second data for the group of individuals, and generating, by the computing system, an integrated data repository that stores the respective portions of the first data and the respective portions of the second data in relation to respective identifiers of the number of identifiers. In other embodiments, the method includes determining, by the computing system, a first set of data processing instructions that are executable in relation to first data stored by the integrated data repository, causing, by the computing system, the first set of data processing instructions to be executed to analyze first health insurance claims codes included in the first data to determine a first subset of the group of individuals in which a biological condition is present and generating, by the computing system, a first dataset indicating the subset of the group of individuals in which the biological condition is present. In other embodiments, the method includes determining, by the computing system, a second set of data processing instructions that are executable in relation to second data stored by the integrated data repository. causing, by the computing system, the second set of data processing instructions to be executed to analyze the second health insurance claims codes included in the second data to determine one or more treatments provided to a second subset of the group of individuals. and generating, by the computing system, a second dataset indicating the one or more treatments provided to the second subset of the group of individuals. In other embodiments, the method includes determining, by the computing system, a third subset of the group of individuals that includes a portion of the first subset of the group of individuals that overlaps with a portion of the second subset of the group of individuals, receiving, by the computing system, a request to perform an analysis of the first dataset and the second dataset in relation to the third subset of the group of individuals, and analyzing, by the computing system and in response to the request, the first dataset and the second dataset with respect to the third subset of the group of individuals to determine a measure of significance of a characteristic of the third subset of the group of individuals with respect to the biological condition.

In other embodiments, the method includes determining, by the computing system, one or more genomic mutations present in the third subset of the group of individuals, determining, by the computing system, a plurality of treatments provided to the third subset of the group of individuals, and determining, by the computing system, respective survival rates for the third subset of the group of individuals. In other embodiments, the measure of significance corresponds to survival rate with respect to a treatment of the plurality of treatments and a genomic mutation of the one or more genomic mutations. In other embodiments, the method includes determining, by the computing system and based on measure of significance, an effectiveness of the treatment for the third subset of the group of individuals. In other embodiments, the method includes determining, by the computing system, individuals in third subset of the group of individuals that have not received the treatment. In other embodiments, the method includes administering one or more therapeutically effective amounts of the treatment to the individuals in the third subset that have not received the treatment. In other embodiments, the integrated data repository is arranged according to a data repository schema that includes a plurality of data tables and a plurality of logical links between the plurality of data tables, individual logical links of the plurality of logical links indicating one or more rows of a data table of the plurality of data tables that correspond to one or more additional rows of an additional data table of the plurality of data tables.

In other embodiments, the plurality of data tables include a first data table that stores genomics data of the group of individuals, a second data that stores data related to one or more patient visits by individuals to one or more healthcare providers, a third data table that stores information corresponding to respective services provided to individuals with respect to one or more patient visits to one or more healthcare providers indicated by the second data table, a fourth data table that stores personal information of the group of individuals, a fifth data table that stores information related to a health insurance company or governmental entity that made payment for services provided to the group of individuals, a sixth data table storing information corresponding to health insurance coverage information for the group of individuals, and a seventh data table that stores information related to pharmaceutical treatments obtained by the group of individuals. In other embodiments, the number of identifiers generated using the second hash function comprise intermediate identifiers; and the method includes applying, by the computing system, a salt function to the intermediate identifiers to generate a final set of identifiers. In other embodiments, the method includes obtaining, by the computing system, information from an additional data repository that includes electronic medical records of an additional group of individuals, determining, by the computing system, a subset of the additional group of individuals that corresponds to the group of individuals having data stored by the genomics data repository, and modifying, by the computing system, the integrated data repository to store at least a portion of the information of the medical records of the subset of the additional group of individuals in relation to the number of identifiers. In other embodiments, the method includes performing, by the computing system, one or more optical character recognition operations with respect to the additional information, analyzing, by the computing system, the additional information obtained from the additional data repository to determine one or more portions of the additional information to remove to produce a corpus of information. In other embodiments, the method includes analyzing, by the computing system, the corpus of information to determine a portion of the subset of the additional group of individuals that correspond to one or more biomarkers, and generating, by the computing system, one or more data structures that store identifiers of the portion of the subset of the additional group of individuals and that store an indication that the portion of the subset of the additional group of individuals corresponds to the one or more biomarkers.

In other embodiments, the method includes storing, by the computing system, the one or more data structures in an intermediate data repository, performing, by the computing system, one or more de-identification operations with respect to the identifiers of the portion of the subset of the additional group of individuals before modifying the integrated data repository to store at least a portion of the additional information of the medical records of the portion of the subset of the additional group of individuals in relation to the number of identifiers. In other embodiments, the molecular data repository stores at least one or more of genomic information, genetic information, metabolomic information, transcriptomic information, fragmentomic information, immune receptor information, methylation information, epigenomic information, or proteomic information. In other embodiments, the method includes determining the likelihood of recurrence includes MRD test, real world evidence (RWE), or both.

### BRIEF DESCRIPTION OF FIGURES

FIG. 1. Clustering samples by methylation markers show potential for methylation signature of MTAP status: MTAP deletion enriched, MTAP depleted, and intermediate clusters Samples with MTAP loss (LoH or Homozgous deletions) trend toward distinct cluster from samples with intact MTAP Minimum >10% TF for all included samples.

FIG. 2. Methylation signature that broadens the population eligible for therapies.

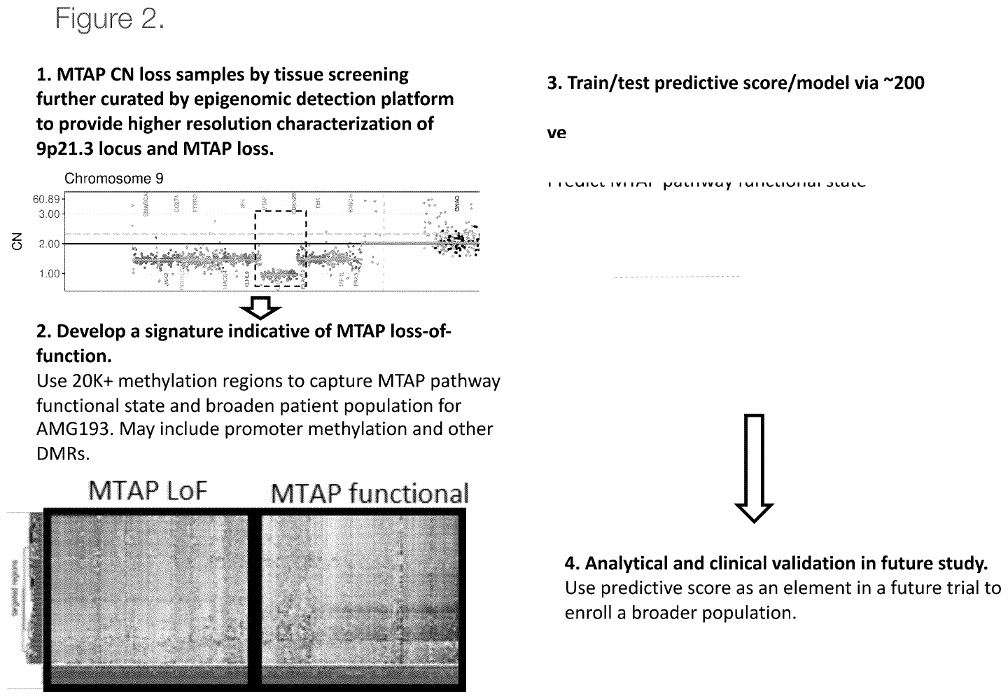

FIG. 3. Predictive Biomarker.

FIG. 4. Increased MTAP deletion in samples with high tumor fraction. Deletion detection is sensitive to tumor fraction. Table below shows the correlation of MTAP deletions wrt Tumor fraction.

FIG. 5. CDKN2A deletion adjacent to intact MTAP gene

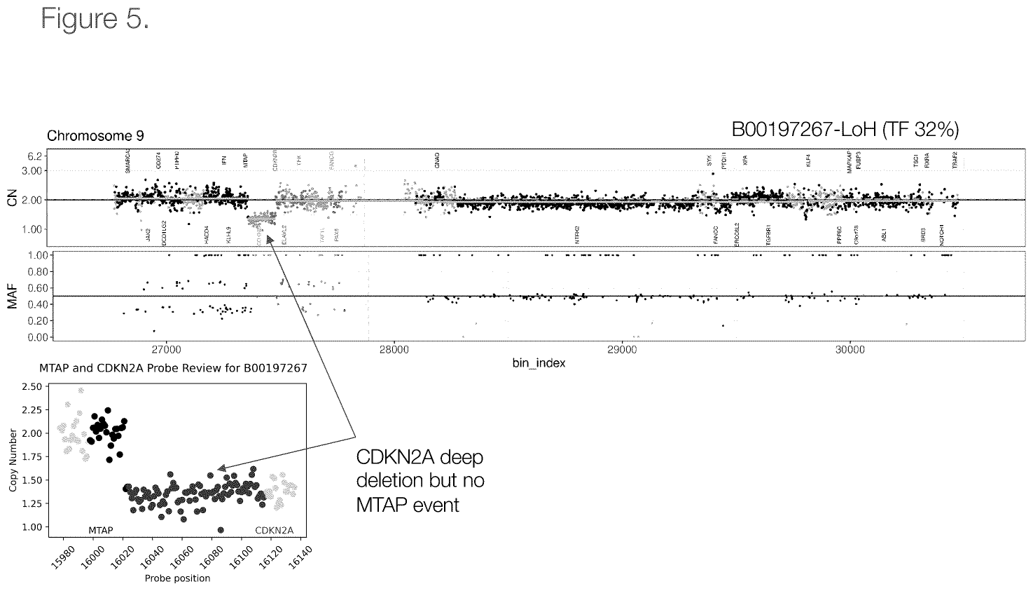

FIG. 6. MTAP loss-of-function detection

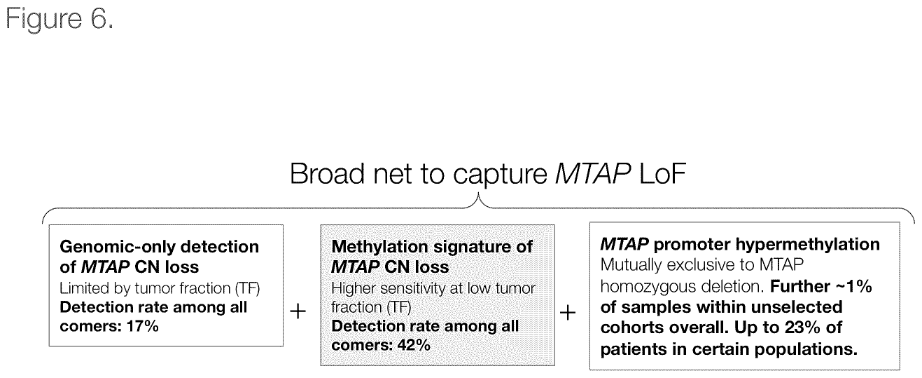

FIG. 7. Samples used to train models for MTAP copy number (CN) loss prediction

FIG. 8. Modeling algorithm flow for MTAP copy number (CN) loss prediction

FIG. 9. Feature Development

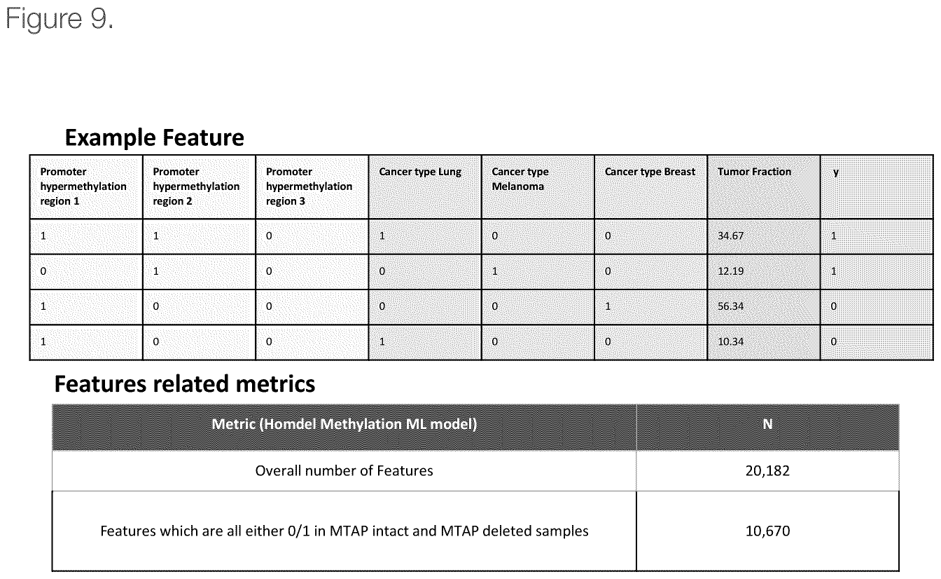

FIG. 10. Data Cleaning for MTAP copy number (CN) loss prediction. Balancing training datasets for cancer type and TF enables us to pick features which can be independent of cancer type

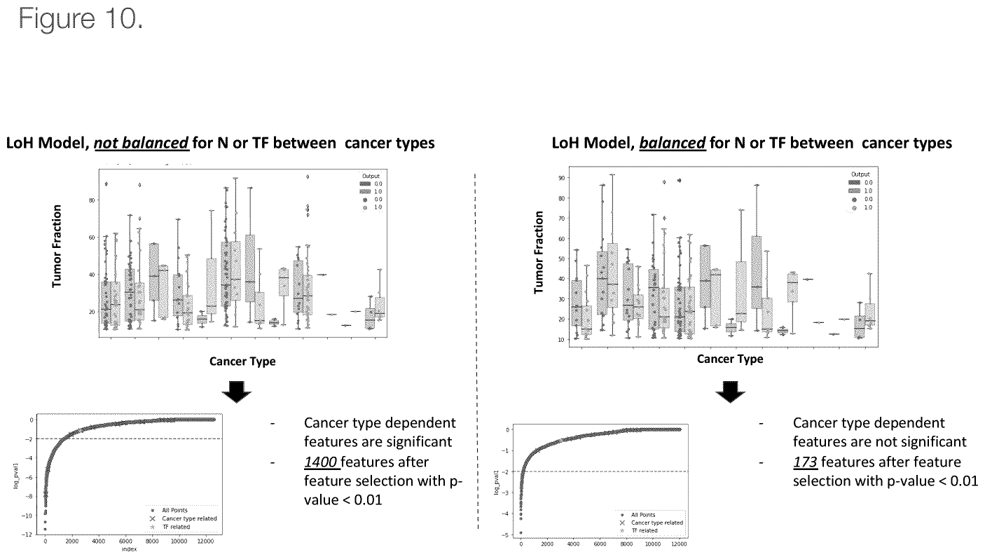

FIG. 11. Data Cleaning for MTAP Homdel copy number (CN) loss prediction

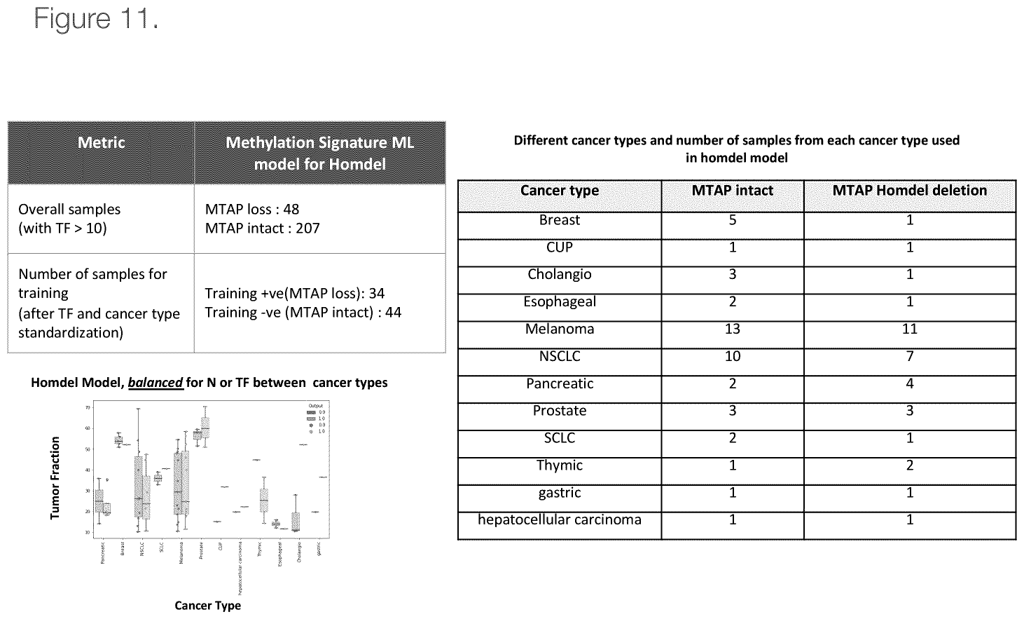

FIG. 12. Feature Selection for MTAP Homdel copy number (CN) loss prediction: Univariate Feature Selection with Logistic Regression. Left panel, Scatter plot visualizes the log-transformed p-values of features obtained from Univariate Feature Selection with Logistic Regression (Logit), Right panel, The plot visualizes the logit coefficients of features with statistically significant values obtained from logistic regression, highlighting the uncertainty with error bars (confidence intervals) N=46 features

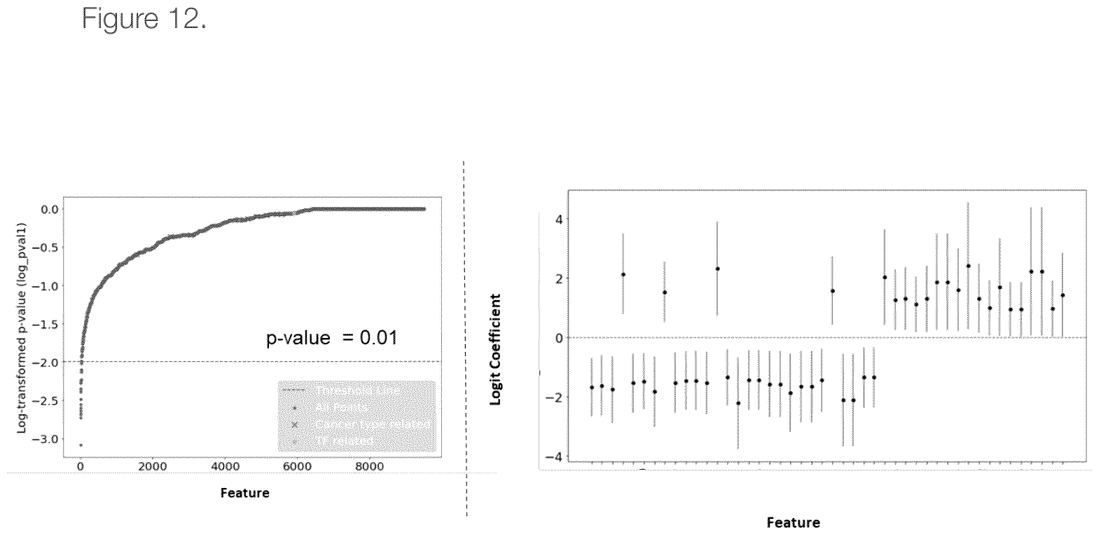

FIG. 13. Model Construction for MTAP Homdel copy number (CN) loss prediction: Logistic Regression model with Elastic Net Regularization. Hyper parameter optimization on C:Regularization helps prevent overfit. Smaller C, means higher penalization of large coefficients and hence stronger regularization. L1_ratio:L1 ratio is a parameter that controls the balance between two types of regularization: L1 regularization (lasso) and L2 regularization (ridge). Smaller L_ratio:model tens to be more L2 than L1. Final model after hyper parameter optimization for accuracy N=46 features

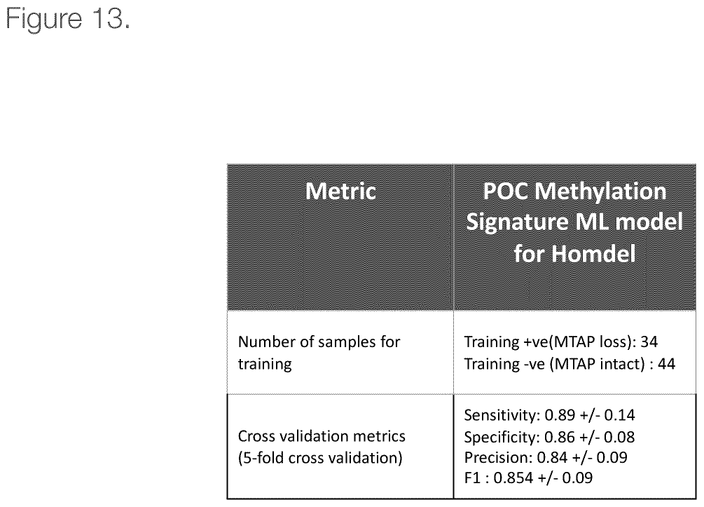

FIG. 14. Model Construction for MTAP Homdel copy number (CN) loss prediction: Logistic Regression model with Elastic Net Regularization. Left panel, Boxplot visualizes the distribution of average logistic regression coefficients for each feature across different folds, providing insights into the variability and central tendency of the coefficient estimates. N=46 features logistic coefficient represents weights assigned to each feature in the model, at each fold. Right panel, Clustered heatmap visualizes the relationships between significant features, identified through logistic regression

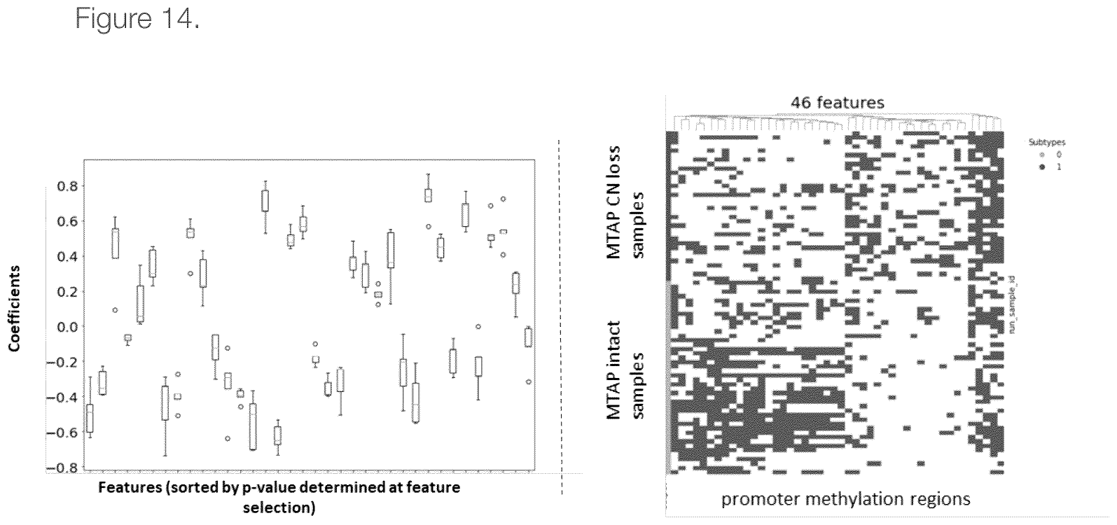

FIG. 15. Conservative number of methylation regions utilized within model in order to avoid overfitting. Left panel, MTAP loss leads to disrupted metabolism/signaling resulting in compensatory mechanisms such as methylation changes which can be used for diagnostic purposes. Right panel, Heatmap of methylation region signal within homozygous deletion model

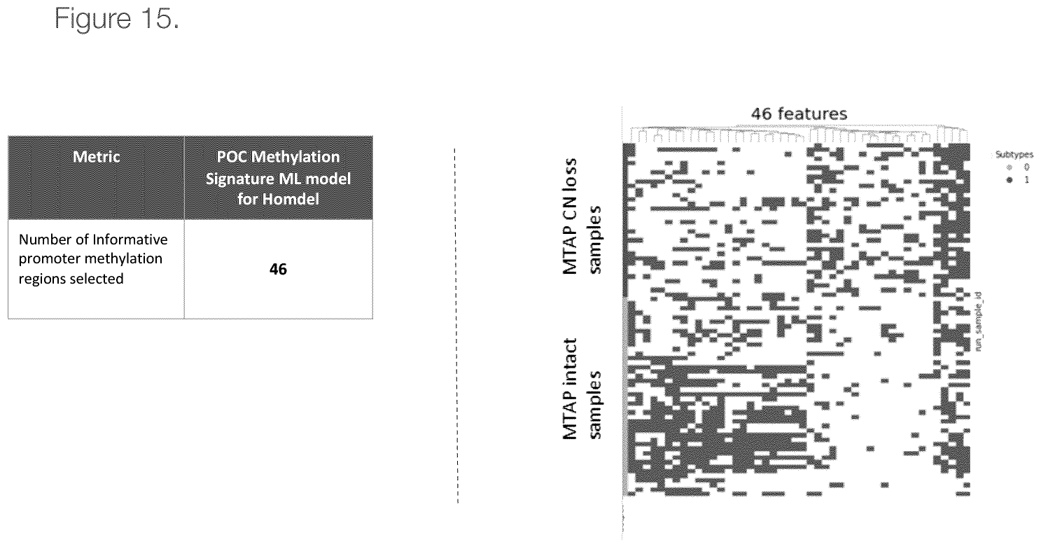

FIG. 16. Within training sensitivity and specificity for MTAP CN loss for homdel prediction is promising

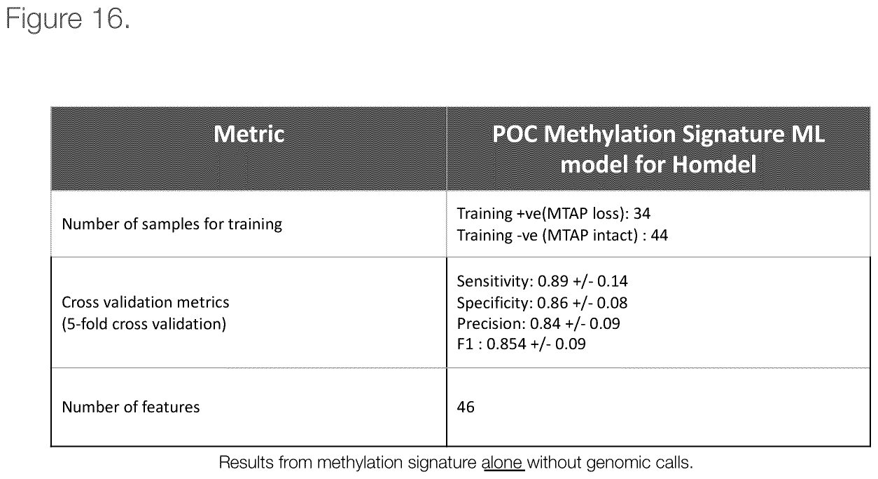

FIG. 17. MTAP methylation signature validation: Specificity 100 self-proclaimed cancer-free samples

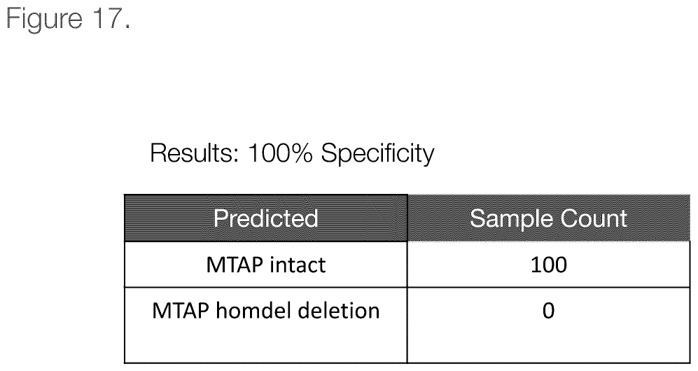

FIG. 18. Sensitivity of homdel methylation signature on development samples. The MTAP deletion methylation signature ML model exhibits heightened sensitivity compared to the genomic caller at lower tumor fractions (TF<10), and comparable sensitivity at higher tumor fractions (TF>10).

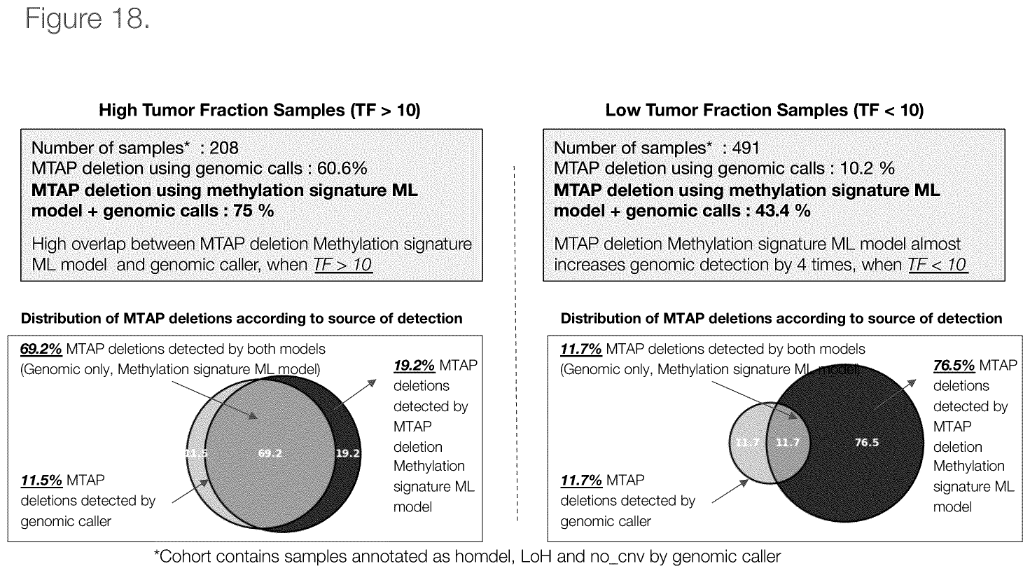

FIG. 19. Biological relevance of the regions observed (Only stringest clusters added here)

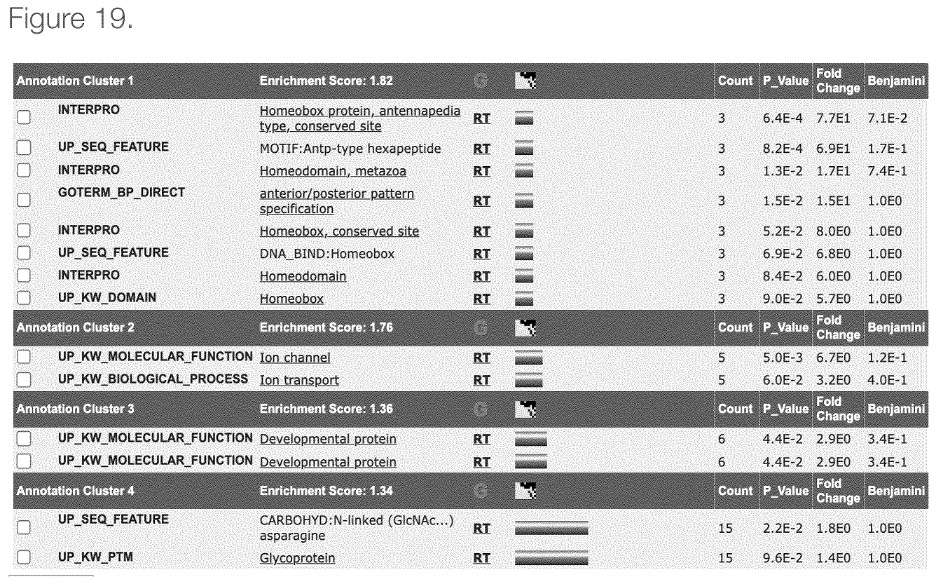

FIG. 20. Liquid Methylation region differential between +ve and −ve. Leveraging Individual DMR regions, to see if there is true enrichment between MTAP intact and MTAP deleted samples. p-values determined by MannWhittneyU Test. Each point corresponds to a DMR region

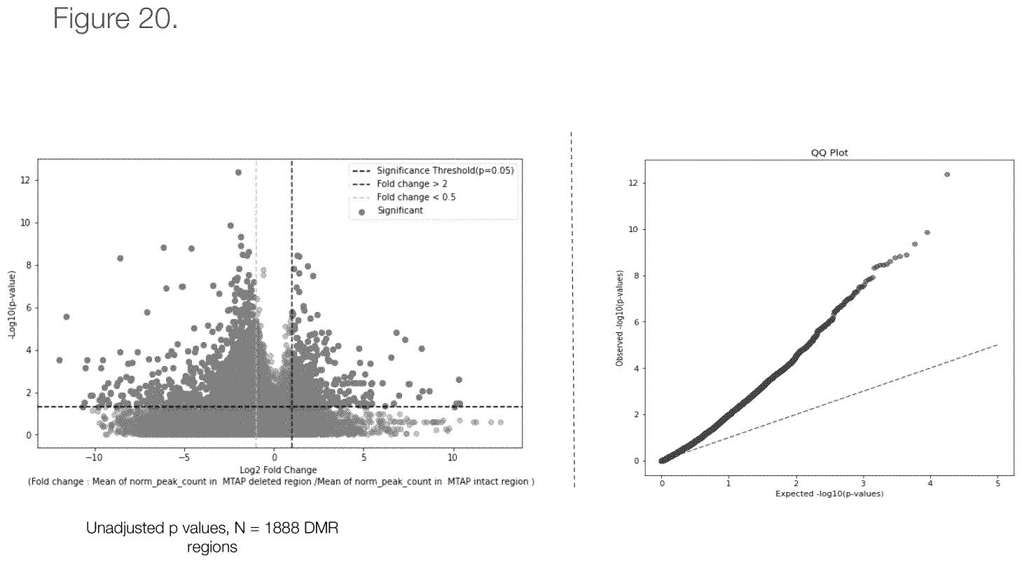

FIG. 21. Liquid Methylation region differential between +ve and −ve. Leveraging Individual DMR regions, to see if there is true enrichment between MTAP intact and MTAP deleted samples. p-values determined by MannWhittneyU Test. Each point corresponds to a DMR region

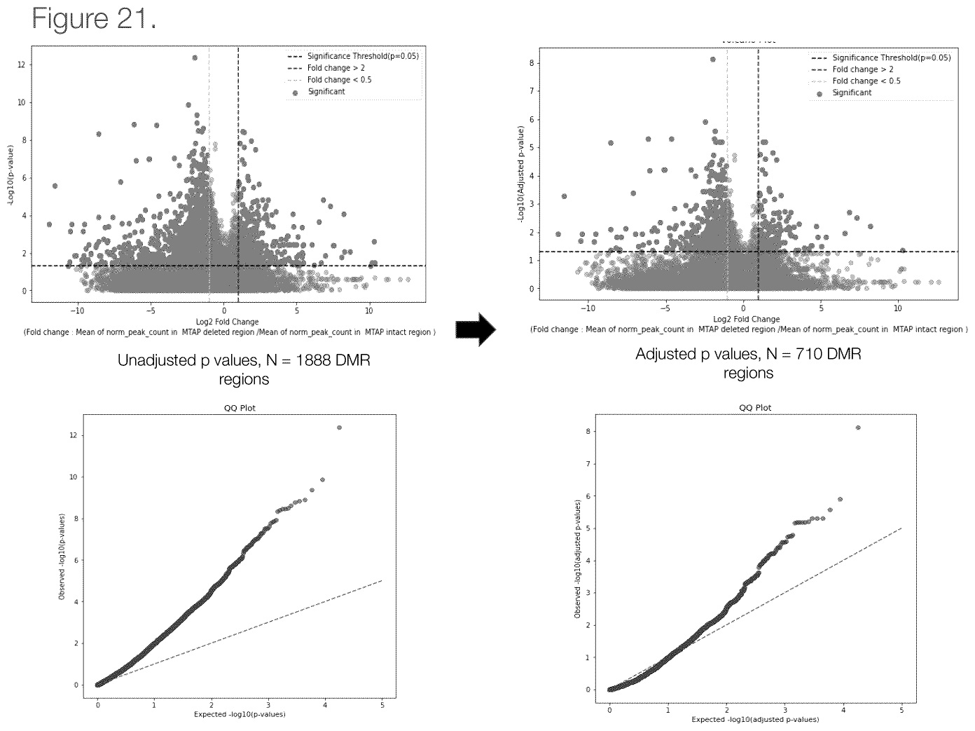

FIG. 22. Liquid Methylation region differential between +ve and −ve. Leveraging Individual DMR regions, to see if there is true enrichment between MTAP intact and MTAP deleted samples. Effect size calculated by hedges g 0—no effect size, 0.2—small effect size, 0.5—medium effect size, 0.8—large effect size.

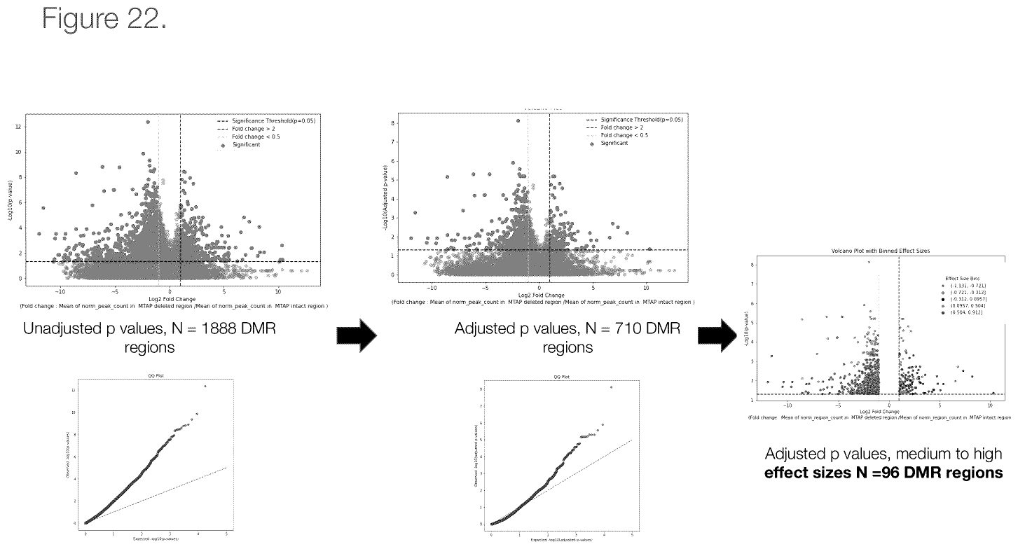

FIG. 23. MTAP deletion DMRs are hypo methylated

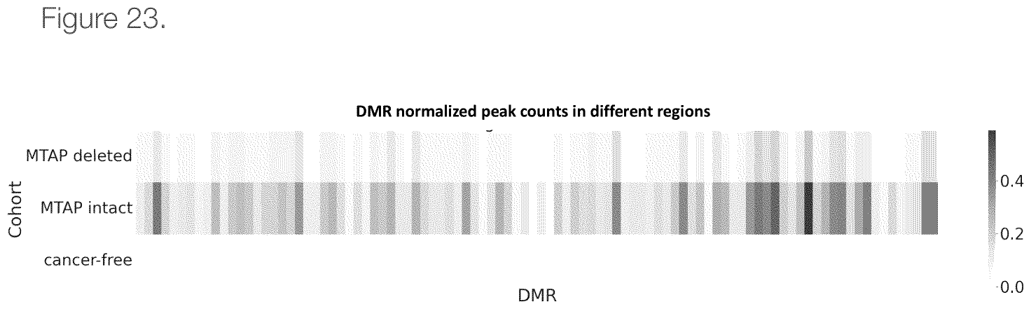

FIG. 24. Some DMRs show dependence on TF. 14/96 DMRs have dependence on Tumor Fraction for MTAP intact and MTAP deleted cohorts

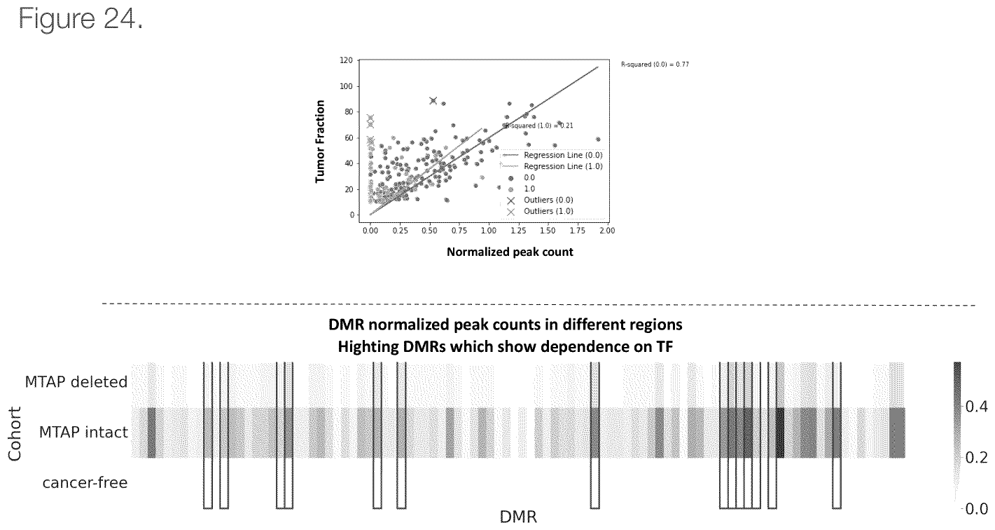

FIG. 25. Some DMRs have high baseline noise. Baseline is calculated from CGP and dev samples. 20/96 DMRs have baseline Coefficient of Variance (CV) and mean to be same as that of MTAP deleted regions

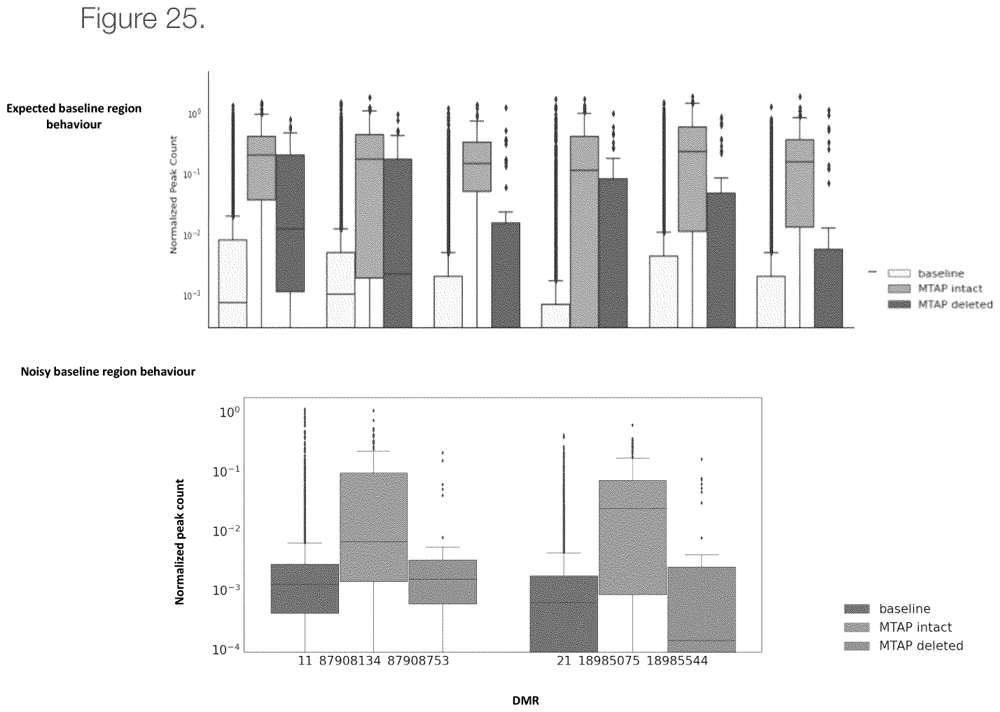

FIG. 26. Some DMRs have high baseline noise. Baseline is calculated from CGP and dev samples. 20/96 DMRs have baseline Coefficient of Variance (CV) and mean to be same as that of MTAP deleted regions

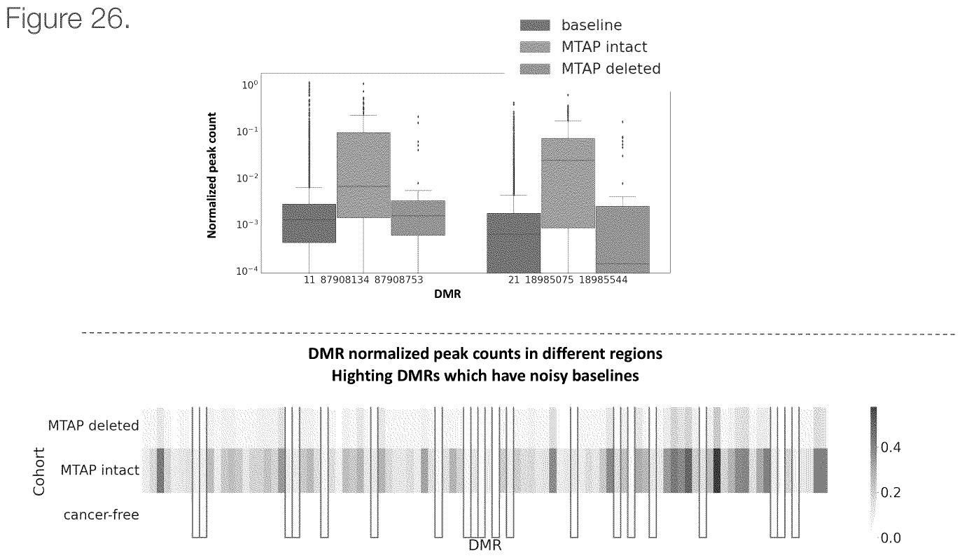

FIG. 27. Biological relevance of the DMRs observed

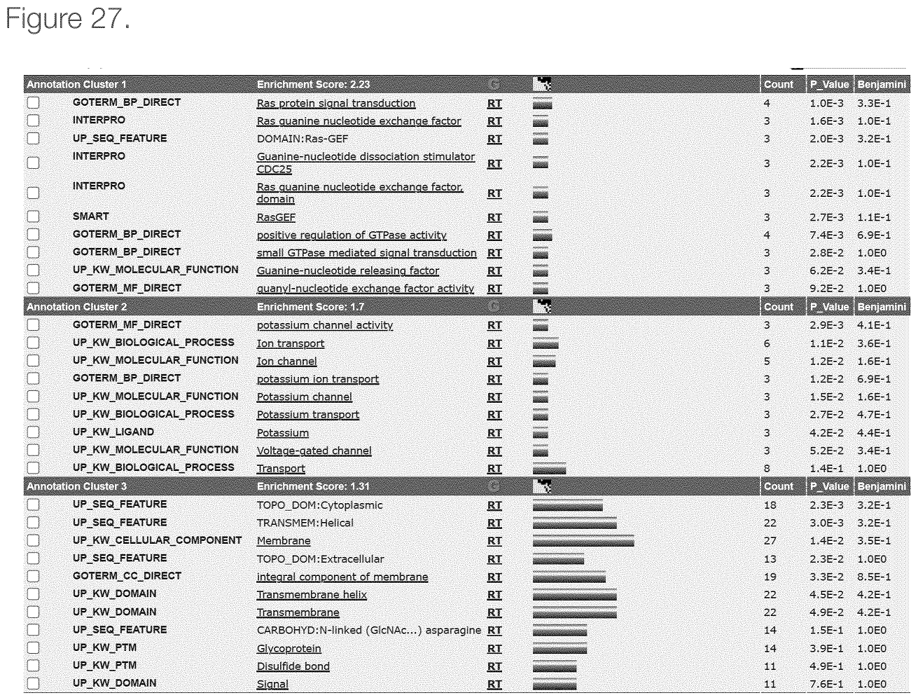

FIG. 28. Biological relevance of the DMRs observed

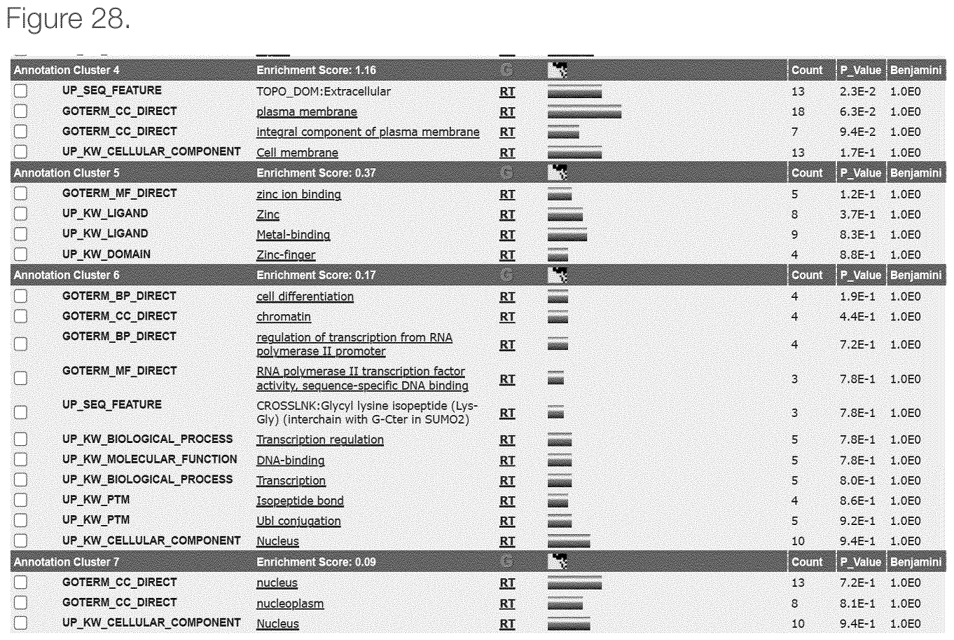

FIG. 29. Observed difference in DMR mean beta values for TCGA cohort. Samples with MTAP homozygous deletion have a higher mean level of methylation that samples with intact MTAP. Left panel, Stronger right-skewed distribution of beta values for 391 DMRs in MTAP-intact samples. Right panel, Fold differences and p-values for 391 DMRs between MTAP-intact and MTAP-homdel samples.

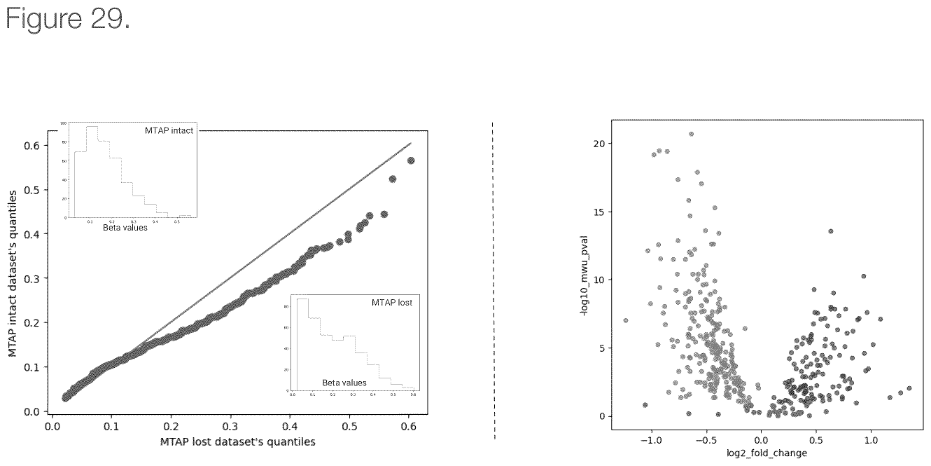

FIG. 30. EGFR mutation variation detection. EGFR activating mutation predictive model development using 47 lung samples, 70 DMRs. Variants included: Exon 19 short dels, Exon 20 short insertions, L858R Variants, G719 Variants, L861Q Variants, Control—no EGFR variants or CNVs. Model performs surprisingly well despite low sample count. Slight TF bias (higher in Lung-non-mut.

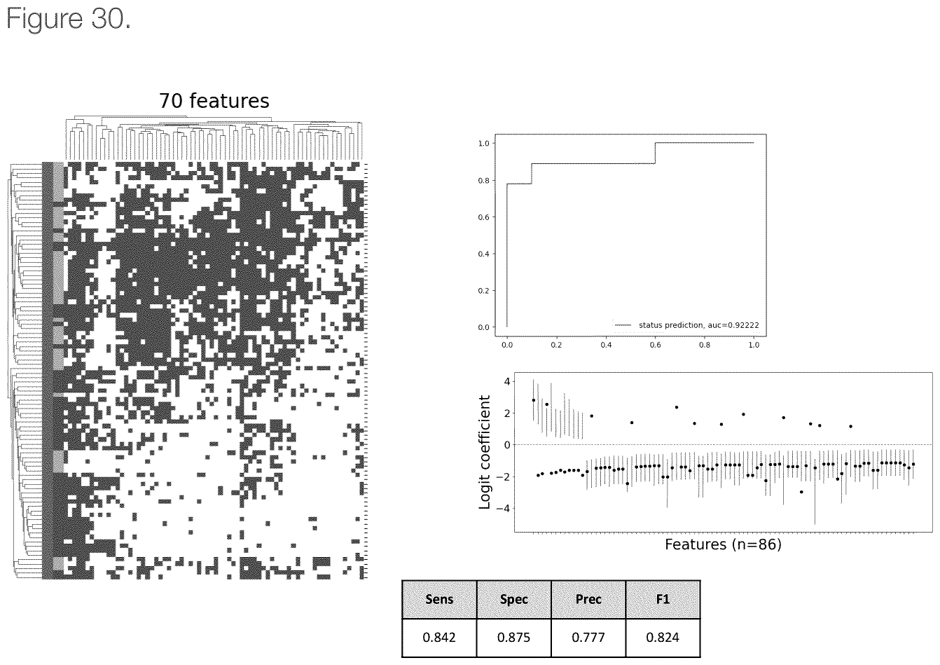

FIG. 31. PTEN model. PTEN model development utilized 186 samples, 256 DMRs, including performing independent hierarchical clustering (ward linkage) and KMeans clustering. Able to observed PIK3CA/AKT1 activating mutations in PCa samples, eEvaluated chromosomal aneuploidy, with potential for correlation with clustering.

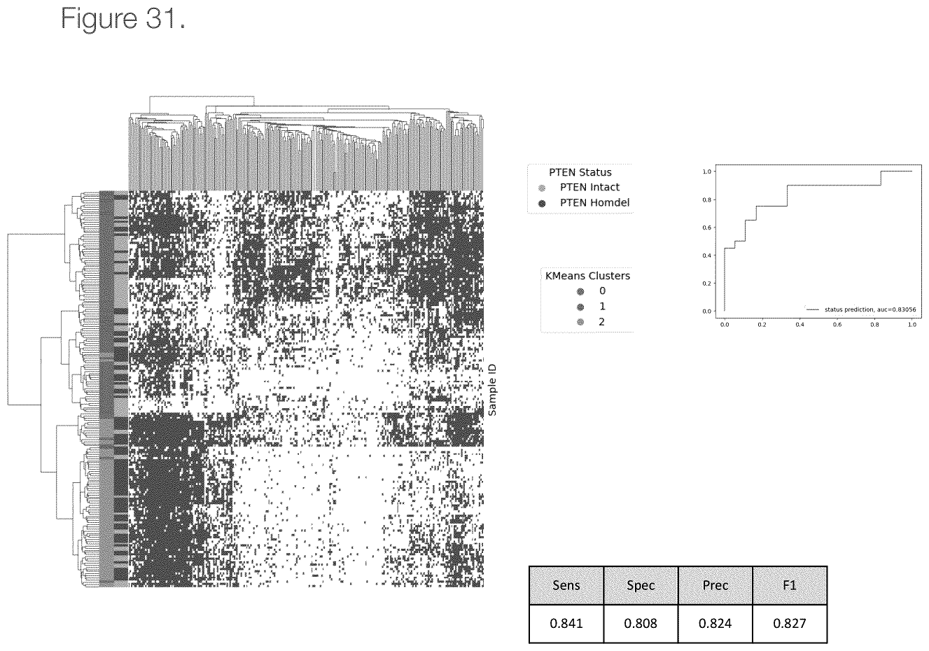

FIG. 32. PTEN models performs better on prostate alone then combined cohort. Further application can be to samples including PCa (186), Lung (70), Breast (66) w/PTEN homdel or intact.

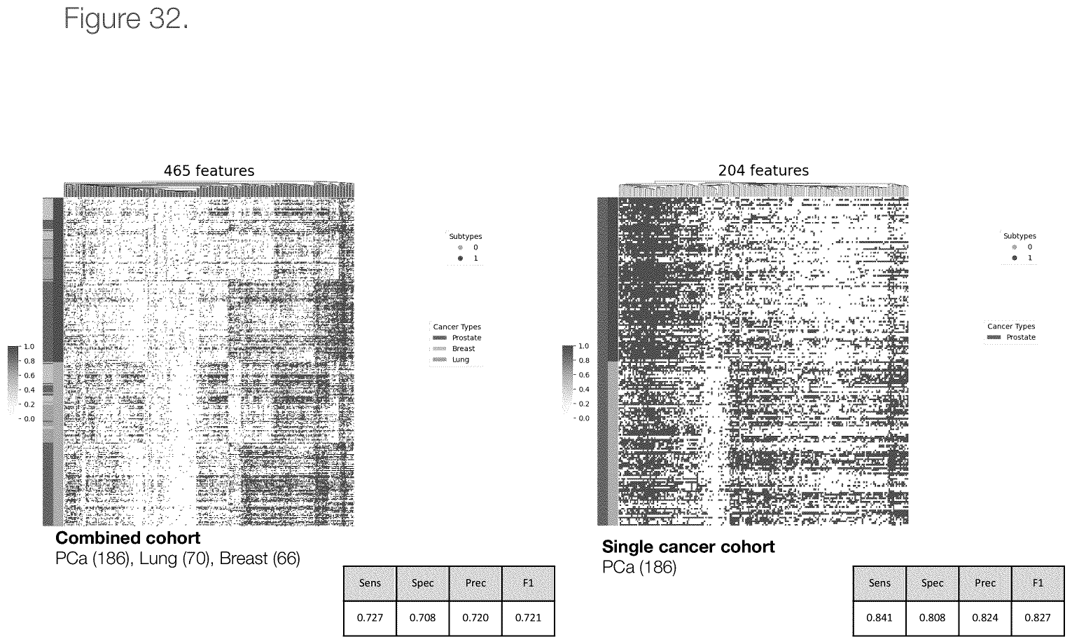

FIG. 33. RB1 model. Prostate cancer model for RB1 deletion prediction functions appropriate, albeit with lower performance than either prostate (PTEN) or bladder (MTAP). Here, 89 features after LR univariate selection and elasticnet penalty applied, for 158 samples (half homdel, half intact). Interestingly, a single cluster 1 stands out in analysis. No TP53 mutations (Clusters 0 and 2 have multiple); lower average degree of aneuploidy. Comparable average TF with other clusters

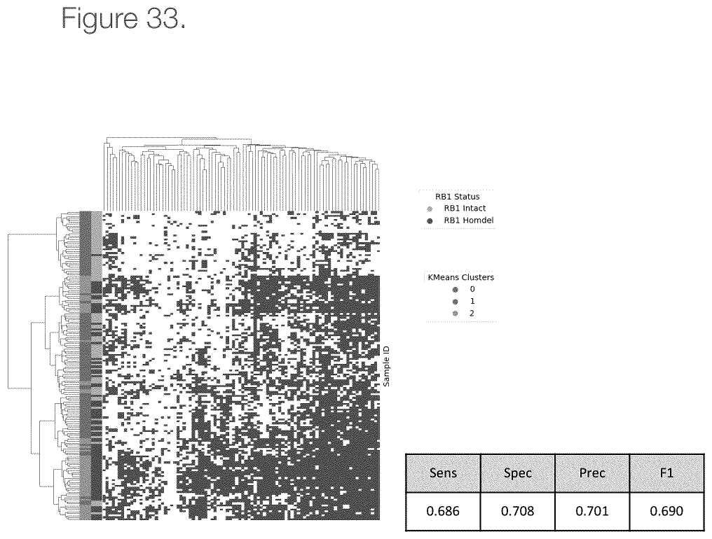

### DETAILED DESCRIPTION

While various embodiments of the disclosure have been shown and described herein, those skilled in the art will understand that such embodiments are provided by way of example only. Numerous variations, changes, and substitutions may occur to those skilled in the art without departing from the disclosure. It should be understood that various alternatives to the embodiments of the disclosure described herein may be employed.

The term “about” and its grammatical equivalents in relation to a reference numerical value can include a range of values up to plus or minus 10% from that value. For example, the amount “about 10” can include amounts from 9 to 11. The term “about” in relation to a reference numerical value can include a range of values plus or minus 10%, 9%, 8%, 7%, 6%, 5%, 4%, 3%, 2%, or 1% from that value.

The term “at least” and its grammatical equivalents in relation to a reference numerical value can include the reference numerical value and greater than that value. For example, the amount “at least 10” can include the value 10 and any numerical value above 10, such as 11, 100, and 1,000.

The term “at most” and its grammatical equivalents in relation to a reference numerical value can include the reference numerical value and less than that value. For example, the amount “at most 10” can include the value 10 and any numerical value under 10, such as 9, 8, 5, 1, 0.5, and 0.1.

As used herein the singular forms “a”, “an”, and “the” can include plural referents unless the context clearly dictates otherwise. Thus, for example, reference to “a cell” can include a plurality of such cells and reference to “the culture” can include reference to one or more cultures and equivalents thereof known to those skilled in the art, and so forth. All technical and scientific terms used herein can have the same meaning as commonly understood to one of ordinary skill in the art to which this disclosure belongs unless clearly indicated otherwise.

Current approaches are to omit testing both genomic and epigenomic attributes of the patient sample or to perform multiple tests separately. Omitting genomic or epigenomic information can result in prescription of cancer therapies that could be known to be ineffective or withholding cancer therapies that could be known to be effective, had both genomic and epigenomic information been available. Cancer can be indicated by epigenetic variations, such as methylation. Examples of methylation changes in cancer include local gains of DNA methylation in the CpG islands at the transcription start site (TSS) of genes involved in normal growth control, DNA repair, cell cycle regulation, and/or cell differentiation. This hypermethylation can be associated with an aberrant loss of transcriptional capacity of involved genes and occurs at least as frequently as point mutations and deletions as a cause of altered gene expression. DNA methylation profiling can be used to detect regions with different extents of methylation (“differentially methylated regions” or “DMRs”) of the genome that are altered during development or that are perturbed by disease, for example, cancer or any cancer-associated disease. The genome of cancer cells harbor imbalance in the above DNA methylation patterns, and therefore in functional packaging of the DNA. The abnormalities of chromatin organization are therefore coupled with methylation changes and may contribute to enhanced cancer profiling when analyzed jointly. Combining MBD-partitioning with fragmentomic data, such as fragment mapped starts and stops positions (correlated with nucleosome positions), fragment length and associated nucleosome occupancy, can be used for chromatin structure analysis in hypermethylation studies with the aim to improve biomarker detection rate.

Methylation profiling can involve determining methylation patterns across different regions of the genome. For example, after partitioning molecules based on extent of methylation (e.g., relative number of methylated sites per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

A characteristic of nucleic acid molecules may be a modification, which may include various chemical or protein modifications (i.e. epigenetic modifications). Non-limiting examples of chemical modification may include, but are not limited to, covalent DNA modifications, including DNA methylation. In some embodiments, DNA methylation includes addition of a methyl group to a cytosine at a CpG site (a cytosine followed by a guanine in a nucleic acid sequence). In some embodiments, DNA methylation includes addition of a methyl group to adenine, such as in N6-methyladenine. In some embodiments, DNA methylation is 5-methylation (modification of the 5th carbon of the 6 carbon ring of cytosine). In some embodiments, 5-methylation includes addition of a methyl group to the 5C position of the cytosine to create 5-methylcytosine (m5c). In some embodiments, methylation includes a derivative of m5c. Derivatives of m5c include, but are not limited to, 5-hydroxymethylcytosine (5-hmC), 5-formylcytosine (5-fC), and 5-carboxylcytosine (5-caC). In some embodiments, DNA methylation is 3C methylation (modification of the 3rd carbon of the 6 carbon ring of cytosine). In some embodiments, 3C methylation includes addition of a methyl group to the 3C position of the cytosine to generate 3-methylcytosine (3mC). Other examples include N6-methyladenine or glycosylation. DNA methylation includes addition of methyl groups to DNA (e.g. CpG) and can change the expression of methylated DNA region. Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer.

A CpG dyad is the dinucleotide CpG (cytosine-phosphate-guanine, i.e. a cytosine followed by a guanine in a 5′→3′ direction of the nucleic acid sequence) on the sense strand and its complementary CpG on the antisense strand of a double-stranded DNA molecule. CpG dyads can be either fully methylated or hemi-methylated (methylated on one strand only).

The CpG dinucleotide is underrepresented in the normal human genome, with the majority of CpG dinucleotide sequences being transcriptionally inert (e.g. DNA heterochromatic regions in pericentromeric parts of the chromosome and in repeat elements) and methylated. However, many CpG islands are protected from such methylation especially around transcription start sites (TSS).

Protein modifications include binding to components of chromatin, particularly histones including modified forms thereof, and binding to other proteins, such as proteins involved in replication or transcription. The disclosure provides methods of processing and analyzing nucleic acids with different extents of modification, such that the nature of their original modification is correlated with a nucleic acid tag and can be decoded by sequencing the tag when nucleic acids are analyzed. Genetic variation of sample nucleic acid modifications can then be associated with the extent of modification (epigenetic variation) of that nucleic acid in the original sample, include single stranded (e.g., ssDNA or RNA) or double stranded molecules (e.g., dsDNA).

The loss of DNA can reduce the presence of one or more types of DNA such that the presence of the one or more types of DNA such as cfDNA, is difficult to detect. In one or more additional scenarios, existing methods to measure DNA methylation, such as enrichment or depletion methods, can have a relatively high level of resolution, such as about 100 base pairs (bp) to about 200 bp that can make accurately determining an amount of methylation of DNA difficult. The accuracy with which DNA methylation is determined can impact the accuracy of estimates of tumor fraction for samples. Since tumor fraction can be used to determine whether a sample is derived from a subject in which a tumor is present or not, the accuracy of determinations of tumor fraction estimates can impact diagnosis and/or treatment decisions for individuals.

MTAP (5′-methylthioadenosine phosphorylase) encodes a key enzyme involved in the metabolism of polyamines and purines. This enzyme converts 5′-methylthioadenosine (MTA), a by-product of polyamine biosynthesis, into adenine and MTR-1-P (methylthioribose-1-phosphate), which are recycled into AMP (adenosine monophosphate) and methionine. This protein is expressed virtually in all tissues throughout the body, and its homozygous deletion is frequently associated with solid and hematologic tumors such as mesothelioma, lung carcinoma, hepatocellular carcinoma, gastrointestinal stromal tumors, metastatic melanoma, leukemias, and lymphoma. Therefore, MTAP has been reported as a tumor suppressor gene; however, many studies have demonstrated the contradictory function of MTAP. For instance, the loss of MTAP expression has been associated with inhibition of growth and progression of head and neck carcinoma and lung cancer by MTA accumulation. Bistulfi et al. showed that knockdown of MTAP blocks prostate cancer growth in vitro and in vivo. In addition, methionine deprivation acts by inhibiting cell migration, invasion, and metastasis in breast cancer. Metabolic changes in tumors, especially those relating to polyamines metabolism, demonstrate that many mechanisms underlying MTAP function need to still be clarified. In gliomas, loss of MTAP locus is also frequently reported. Nevertheless, the clinical and the biological impacts of MTAP are poorly explored in gliomas.

Mutation of MTAP results in dysregulated epigenetics and cancer cell stemness. Based on this functional basis, therapeutic strategies have been developed to target MTAP loss for cancer treatment. Firstly, in MTAP-deficient tumor cells, the absence of the salvage pathway sensitizes cells to inhibitors of de novo purine synthesis and provides an opportunity to specifically target cancer cells. Secondly, MTAP loss results in the accumulation of MTA and this inhibits the activity of several enzymes, including protein arginine methyltransferase 5 (PRMT5). PRMT5 is a regulatory protein critical for multiple processes such as genome organization, cell cycle regulation, and stem cell differentiation. In addition, dysregulation of PRMT5 is associated with multiple cancers and neurological disorders. Deletion of MTAP results in increased dependency on PRMT5 in cancer cells thus providing the potential avenues for targeted therapies. Besides PRMT5, the vulnerability is also noticed in other upstream and downstream enzymes of PRMT5 including methionine adenosyltransferase II alpha (MAT2A) and Rio domain containing protein (RIOK1).

Although MTAP is functionally relevant in several tumors, MTAP has not been studied extensively and this is evident from the number of published papers in literature databases.

### Samples

A sample can be any biological sample isolated from a subject. A sample can be a bodily sample. Samples can include body tissues, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, a preferred body fluid for analysis is plasma or serum containing cell-free nucleic acids. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer. The subject may not have cancer or a detectable cancer symptom. The subject may have been treated with one or more cancer therapy, e.g., any one or more of chemotherapies, antibodies, vaccines or biologics. The subject may be in remission. The subject may or may not be diagnosed as being susceptible to cancer or any cancer-associated genetic mutations/disorders.

The volume of plasma can depend on the desired read depth for sequenced regions. Exemplary volumes are 0.4-40 ml, 5-20 ml, 10-20 ml. For example, the volume can be 0.5 mL, 1 mL, 5 mL 10 mL, 20 mL, 30 mL, or 40 mL. The volume of sampled plasma may be 5 to 20 mL.

A sample can comprise various amounts of nucleic acid that contains genome equivalents. For example, a sample of about 30 ng DNA can contain about 10,000 (104) haploid human genome equivalents and, in the case of cfDNA, about 200 billion (2×1011) individual polynucleotide molecules. Similarly, a sample of about 100 ng of DNA can contain about 30,000 haploid human genome equivalents and, in the case of cfDNA, about 600 billion individual molecules.

A sample can comprise nucleic acids from different sources, e.g., from cells and cell-free of the same subject, from cells and cell-free of different subjects. A sample can comprise nucleic acids carrying mutations. For example, a sample can comprise DNA carrying germline mutations and/or somatic mutations. Germline mutations refer to mutations existing in germline DNA of a subject. Somatic mutations refer to mutations originating in somatic cells of a subject, e.g., cancer cells. A sample can comprise DNA carrying cancer-associated mutations (e.g., cancer-associated somatic mutations). A sample can comprise an epigenetic variant (i.e. a chemical or protein modification), wherein the epigenetic variant is associated with the presence of a genetic variant such as a cancer-associated mutation. In some embodiments, the sample includes an epigenetic variant associated with the presence of a genetic variant, wherein the sample does not comprise the genetic variant.

Exemplary amounts of cell-free nucleic acids in a sample before amplification range from about 1 fg to about 1 g, e.g., 1 μg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of cell-free nucleic acid molecules. The amount can be at least 1 fg, at least 10 fg, at least 100 fg, at least 1 pg, at least 10 pg, at least 100 pg, at least 1 ng, at least 10 ng, at least 100 ng, at least 150 ng, or at least 200 ng of cell-free nucleic acid molecules. The amount can be up to 1 femtogram (fg), 10 fg, 100 fg, 1 picogram (pg), 10 μg, 100 pg, 1 ng, 10 ng, 100 ng, 150 ng, or 200 ng of cell-free nucleic acid molecules. The method can comprise obtaining 1 femtogram (fg) to 200 ng.

Cell-free nucleic acids are nucleic acids not contained within or otherwise bound to a cell or in other words nucleic acids remaining in a sample after removing intact cells. Cell-free nucleic acids include DNA, RNA, and hybrids thereof, including genomic DNA, mitochondrial DNA, siRNA, miRNA, circulating RNA (cRNA), tRNA, rRNA, small nucleolar RNA (snoRNA), Piwi-interacting RNA (piRNA), long non-coding RNA (long ncRNA), or fragments of any of these. Cell-free nucleic acids can be double-stranded, single-stranded, or a hybrid thereof. A cell-free nucleic acid can be released into bodily fluid through secretion or cell death processes, e.g., cellular necrosis and apoptosis. Some cell-free nucleic acids are released into bodily fluid from cancer cells e.g., circulating tumor DNA, (ctDNA). Others are released from healthy cells. In some embodiments, cfDNA is cell-free fetal DNA (cffDNA) In some embodiments, cell free nucleic acids are produced by tumor cells. In some embodiments, cell free nucleic acids are produced by a mixture of tumor cells and non-tumor cells.

Cell-free nucleic acids have an exemplary size distribution of about 100-500 nucleotides, with molecules of 110 to about 230 nucleotides representing about 90% of molecules, with a mode of about 168 nucleotides and a second minor peak in a range between 240 to 440 nucleotides. Cell-free nucleic acids can be isolated from bodily fluids through a fractionation or partitioning step in which cell-free nucleic acids, as found in solution, are separated from intact cells and other non-soluble components of the bodily fluid. Partitioning may include techniques such as centrifugation or filtration. Alternatively, cells in bodily fluids can be lysed and cell-free and cellular nucleic acids processed together. Generally, after addition of buffers and wash steps, nucleic acids can be precipitated with alcohol. Further clean up steps may be used such as silica based columns to remove contaminants or salts. Non-specific bulk carrier nucleic acids, such as Cot-1 DNA, DNA or protein for bisulfite sequencing, hybridization, and/or ligation, may be added throughout the reaction to optimize certain aspects of the procedure such as yield.

After such processing, samples can include various forms of nucleic acid including double stranded DNA, single stranded DNA and single stranded RNA. In some embodiments, single stranded DNA and RNA can be converted to double stranded forms so they are included in subsequent processing and analysis steps.

### Analytes

Analytes can include nucleic acid analytes, and non-nucleic acid analytes. The disclosure provides for detecting genetic variations in biological samples from a subject. Biological samples may include polynucleotides from cancer cells. Polynucleotides may be DNA (e.g., genomic DNA, cDNA), RNA (e.g., mRNA, small RNAs), or any combination thereof. Biological samples may include tumor tissue, e.g., from a biopsy. In some cases, biological samples may include blood or saliva. In particular cases, biological samples may comprise cell free DNA (“cfDNA”) or circulating tumor DNA (“ctDNA”). Cell free DNA can be present in, e.g., blood.

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquity lati on variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. This further includes receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

In general, the systems, apparatus, methods, and compositions can be used to analyze any number of analytes, further including both nucleic acid analytes and non-nucleic acid analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate. Methods for performing multiplexed assays to analyze two or more different analytes will be discussed in a subsequent section of this disclosure.

One or more nucleic acid analytes and/or non-nucleic acid analytes constitute a set of molecular interactions in a biological system under study (e.g., cells), which may be regarded as “interactome”—the molecular interactions that occur between molecules belonging to different biochemical families (proteins, nucleic acids, lipids, carbohydrates, etc.) and also within a given family. In various embodiments, an interactome is a protein-DNA interactome (network formed by transcription factors (and DNA or chromatin regulatory proteins) and their target genes. In other embodiments, interactome refers to protein-protein interaction network (PPI), or protein interaction network (PIN). The methods described herein allow for study and analysis of the interactome. Techniques such as proteogenomics (whole genome sequencing, whole exome sequencing and RNA-seq, and mass spectrometry as examples) can support study of the interactome.

### Analysis

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic and other analyte data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

The present analyses are also useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy. Additionally, if a cancer is observed to be in remission after treatment, the present methods can be used to monitor residual disease or recurrence of disease.

The present methods can also be used for detecting genetic variations in conditions other than cancer. Immune cells, such as B cells, may undergo rapid clonal expansion upon the presence of certain diseases. Clonal expansions may be monitored using copy number variation detection and certain immune states may be monitored. In this example, copy number variation analysis may be performed over time to produce a profile of how a particular disease may be progressing. Copy number variation or even rare mutation detection may be used to determine how a population of pathogens changes during the course of infection. This may be particularly important during chronic infections, such as HIV/AIDS or Hepatitis infections, whereby viruses may change life cycle state and/or mutate into more virulent forms during the course of infection. The present methods may be used to determine or profile rejection activities of the host body, as immune cells attempt to destroy transplanted tissue to monitor the status of transplanted tissue as well as altering the course of treatment or prevention of rejection.

Further, the methods of the disclosure may be used to characterize the heterogeneity of an abnormal condition in a subject. Such methods can include, e.g., generating a genetic profile of extracellular polynucleotides derived from the subject, wherein the genetic profile includes a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate or profile, fingerprint or set of data that is a summation of genetic information derived from different cells in a heterogeneous disease. This set of data may comprise copy number variation and mutation analyses alone or in combination.

The present methods can be used to diagnose, prognose, monitor or observe cancers. or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

### Determination of 5-Methylcytosine Pattern of Nucleic Acids

Bisulfite-based sequencing and variants thereof provides a means of determining the methylation pattern of a nucleic acid. In some embodiments, determining the methylation pattern includes distinguishing 5-methylcytosine (5mC) from non-methylated cytosine. In some embodiments, determining methylation pattern includes distinguishing N6-methyladenine from non-methylated adenine. In some embodiments, determining the methylation pattern includes distinguishing 5-hydroxymethylcytosine (5hmC), 5-formylcytosine (5fC), and 5-carboxylcytosine (5caC) from non-methylated cytosine. Examples of bisulfite sequencing include, but are not limited to oxidative bisulfite sequencing (OX-BS-seq), Tet-assisted bisulfite sequencing (TAB-seq), and reduced bisulfite sequencing (redBS-seq).

Oxidative bisulfite sequencing (OX-BS-seq) is used to distinguish between 5mC and 5hmC, by first converting the 5hmC to 5fC, and then proceeding with bisulfite sequencing as previously described. Tet-assisted bisulfite sequencing (TAB-seq) can also be used to distinguish 5mc and 5hmC. In TAB-seq, 5hmC is protected by glucosylation. A Tet enzyme is then used to convert 5mC to 5caC before proceeding with bisulfite sequencing, as previously described. Reduced bisulfite sequencing is used to distinguish 5fC from modified cytosines.

Generally, in bisulfite sequencing, a nucleic acid sample is divided into two aliquots and one aliquot is treated with bisulfite. The bisulfite converts native cytosine and certain modified cytosine nucleotides (e.g. 5-formylcytosine or 5-carboxylcytosine) to uracil whereas other modified cytosines (e.g., 5-methylcytosine, 5-hydroxylmethylcystosine) are not converted. Comparison of nucleic acid sequences of molecules from the two aliquots indicates which cytosines were and were not converted to uracils. Consequently, cytosines which were and were not modified can be determined. The initial splitting of the sample into two aliquots is disadvantageous for samples containing only small amounts of nucleic acids, and/or composed of heterogeneous cell/tissue origins such as bodily fluids containing cell-free DNA.

The present disclosure provides methods allowing bisulfite sequencing and variants thereof. These methods work by linking nucleic acids in a population to a capture moiety, i.e., a label that can be captured or immobilized. Capture moieties include, without limitation, biotin, avidin, streptavidin, a nucleic acid including a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase. Following linking of capture moieties to sample nucleic acids, the sample nucleic acids serve as templates for amplification. Following amplification, the original templates remain linked to the capture moieties, but amplicons are not linked to capture moieties.

The capture moiety can be linked to sample nucleic acids as a component of an adapter, which may also provide amplification and/or sequencing primer binding sites. In some methods, sample nucleic acids are linked to adapters at both ends, with both adapters bearing a capture moiety. Preferably any cytosine residues in the adapters are modified, such as by 5methylcytosine, to protect against the action of bisulfite. In some instances, the capture moieties are linked to the original templates by a cleavable linkage (e.g., photocleavable desthiobiotin-TEG or uracil residues cleavable with USER™ enzyme, Chem. Commun. (Camb). 2015 Feb. 21; 51(15): 3266-3269), in which case the capture moieties can, if desired, be removed.

The amplicons are denatured and contacted with an affinity reagent for the capture tag. Original templates bind to the affinity reagent whereas nucleic acid molecules resulting from amplification do not. Thus, the original templates can be separated from nucleic acid molecules resulting from amplification.

Following separation or partition, the respective populations of nucleic acids (i.e., original templates and amplification products) can be subjected to bisulfite treatment with the original template population receiving bisulfite treatment and the amplification products not. Alternatively, the amplification products can be subjected to bisulfite treatment and the original template population is not. Following such treatment, the respective populations can be amplified (which in the case of the original template population converts uracils to thymines). The populations can also be subjected to biotin probe hybridization for enrichment. The respective populations are then analyzed and sequences compared to determine which cytosines were 5-methylated (or 5-hydroxylmethylated) in the original. Detection of a T nucleotide in the template population (corresponding to an unmethylated cytosine converted to uracil) and a C nucleotide at the corresponding position of the amplified population indicates an unmodified C. The presence of C's at corresponding positions of the original template and amplified populations indicates a modified C in the original sample.

In some embodiments, a method uses sequential DNA-seq and bisulfite-seq (BIS-seq) NGS library preparation of molecular tagged DNA libraries. This process is performed by labeling of adapters (e.g., biotin), DNA-seq amplification of whole library, parent molecule recovery (e.g. streptavidin bead pull down), bisulfite conversion and BIS-seq. In some embodiments, the method identifies 5-methylcytosine with single-base resolution, through sequential NGS-preparative amplification of parent library molecules with and without bisulfite treatment. This can be achieved by modifying the 5-methyl-ated NGS-adapters (directional adapters; Y-shaped/forked with 5-methylcytosine replacing) used in BIS-seq with a label (e.g., biotin) on one of the two adapter strands. Sample DNA molecules are adapter ligated, and amplified (e.g., by PCR). As only the parent molecules will have a labeled adapter end, they can be selectively recovered from their amplified progeny by label-specific capture methods (e.g., streptavidin-magnetic beads). As the parent molecules retain 5-methylation marks, bisulfite conversion on the captured library will yield single-base resolution 5-methylation status upon BIS-seq, retaining molecular information to corresponding DNA-seq. In some embodiments, the bisulfite treated library can be combined with a non-treated library prior to enrichment/NGS by addition of a sample tag DNA sequence in standard multiplexed NGS workflow. As with BIS-seq workflows, bioinformatics analysis can be carried out for genomic alignment and 5-methylated base identification. In sum, this method provides the ability to selectively recover the parent, ligated molecules, carrying 5-methylcytosine marks, after library amplification, thereby allowing for parallel processing for bisulfite converted DNA. This overcomes the destructive nature of bisulfite treatment on the quality/sensitivity of the DNA-seq information extracted from a workflow. With this method, the recovered ligated, parent DNA molecules (via labeled adapters) allow amplification of the complete DNA library and parallel application of treatments that elicit epigenetic DNA modifications. The present disclosure discusses the use of BIS-seq methods to identify cytosine5-methylation (5-methylcytosine), but this is not limiting. Variants of BIS-seq have been developed to identify hydroxymethylated cytosines (5hmC; OX-BS-seq, TAB-seq), formylcytosine (5fC; redBS-seq) and carboxylcytosines. These methodologies can be implemented with the sequential/parallel library preparation described herein.

### Alternative Methods of Modified Nucleic Acid Analysis

The disclosure provides alternative methods for analyzing modified nucleic acids (e.g., methylated, linked to histones and other modifications discussed above). In some such methods, a population of nucleic acids bearing the modification to different extents (e.g., 0, 1, 2, 3, 4, 5 or more methyl groups per nucleic acid molecule) is contacted with adapters before fractionation of the population depending on the extent of the modification. Adapters attach to either one end or both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. Following attachment of adapters, the nucleic acids are amplified from primers binding to the primer binding sites within the adapters. Adapters, whether bearing the same or different tags, can include the same or different primer binding sites, but preferably adapters include the same primer binding site. Following amplification, the nucleic acids are contacted with an agent that preferably binds to nucleic acids bearing the modification (such as the previously described such agents). The nucleic acids are separated into at least two partitions differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. Following separation, the different partitions can then be subject to further processing steps, which typically include further amplification, and sequence analysis, in parallel but separately. Sequence data from the different partitions can then be compared.

Nucleic acids can be linked at both ends to Y-shaped adapters including primer binding sites and tags. The molecules are amplified. The amplified molecules are then fractionated by contact with an antibody preferentially binding to 5-methylcytosine to produce two partitions. One partition includes original molecules lacking methylation and amplification copies having lost methylation. The other partition includes original DNA molecules with methylation. The two partitions are then processed and sequenced separately with further amplification of the methylated partition. The sequence data of the two partitions can then be compared. In this example, tags are not used to distinguish between methylated and unmethylated DNA but rather to distinguish between different molecules within these partitions so that one can determine whether reads with the same start and stop points are based on the same or different molecules.

The disclosure provides further methods for analyzing a population of nucleic acid in which at least some of the nucleic acids include one or more modified cytosine residues, such as 5-methylcytosine and any of the other modifications described previously. In these methods, the population of nucleic acids is contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are treated with bisulfite. This treatment converts unmodified cytosines to uracils. The bisulfite treated nucleic acids are then subjected to amplification primed by primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Partitioning the Sample into a Plurality of Subsamples; Aspects of Samples; Analysis of Epigenetic Characteristics

In certain embodiments described herein, a population of different forms of nucleic acids (e.g., hypermethylated and hypomethylated DNA in a sample, such as a captured set of cfDNA as described herein) can be physically partitioned based on one or more characteristics of the nucleic acids prior to further analysis, e.g., differentially modifying or isolating a nucleobase, tagging, and/or sequencing. This approach can be used to determine, for example, whether certain sequences are hypermethylated or hypomethylated. In some embodiments, hypermethylation variable epigenetic target regions are analyzed to determine whether they show hypermethylation characteristic of tumor cells and/or hypomethylation variable epigenetic target regions are analyzed to determine whether they show hypomethylation characteristic of tumor cells. Additionally, by partitioning a heterogeneous nucleic acid population, one may increase rare signals, e.g., by enriching rare nucleic acid molecules that are more prevalent in one fraction (or partition) of the population. For example, a genetic variation present in hyper-methylated DNA but less (or not) in hypomethylated DNA can be more easily detected by partitioning a sample into hyper-methylated and hypo-methylated nucleic acid molecules. By analyzing multiple fractions of a sample, a multi-dimensional analysis of a single locus of a genome or species of nucleic acid can be performed and hence, greater sensitivity can be achieved.

In some instances, a heterogeneous nucleic acid sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristics (examples provided herein) and tagged using differential tags that are distinguished from other partitions and partitioning means.

Examples of characteristics that can be used for partitioning include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some instances, each partition (representative of a different nucleic acid form) is differentially labelled, and the partitions are pooled together prior to sequencing. In other instances, the different forms are separately sequenced. In some embodiments, a population of different nucleic acids is partitioned into two or more different partitions. Each partition is representative of a different nucleic acid form, and a first partition (also referred to as a subsample) includes DNA with a cytosine modification in a greater proportion than a second subsample. Each partition is distinctly tagged. The first subsample is subjected to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. The tagged nucleic acids are pooled together prior to sequencing. Sequence reads are obtained and analyzed, including to distinguish the first nucleobase from the second nucleobase in the DNA of the first subsample, in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Samples can include nucleic acids varying in modifications including post-replication modifications to nucleotides and binding, usually noncovalently, to one or more proteins.

In an embodiment, the population of nucleic acids is one obtained from a serum, plasma or blood sample from a subject suspected of having neoplasia, a tumor, or cancer or previously diagnosed with neoplasia, a tumor, or cancer. The population of nucleic acids includes nucleic acids having varying levels of methylation. Methylation can occur from any one or more post-replication or transcriptional modifications. Post-replication modifications include modifications of the nucleotide cytosine, particularly at the 5-position of the nucleobase, e.g., 5-methylcytosine, 5-hydroxymethylcytosine, 5-formylcytosine and 5-carboxylcytosine. The affinity agents can be antibodies with the desired specificity, natural binding partners or variants thereof (Bock et al., Nat Biotech 28: 1106-1114 (2010); Song et al., Nat Biotech 29: 68-72 (2011)), or artificial peptides selected e.g., by phage display to have specificity to a given target.

Examples of capture moieties contemplated herein include methyl binding domain (MBDs) and methyl binding proteins (MBPs) as described herein, including proteins such as MeCP2 and antibodies preferentially binding to 5-methylcytosine. Likewise, partitioning of different forms of nucleic acids can be performed using histone binding proteins which can separate nucleic acids bound to histones from free or unbound nucleic acids. Examples of histone binding proteins that can be used in the methods disclosed herein include RBBP4, RbAp48 and SANT domain peptides. Although for some affinity agents and modifications, binding to the agent may occur in an essentially all or none manner depending on whether a nucleic acid bears a modification, the separation may be one of degree. In such instances, nucleic acids overrepresented in a modification bind to the agent at a greater extent that nucleic acids underrepresented in the modification. Alternatively, nucleic acids having modifications may bind in an all or nothing manner. But then, various levels of modifications may be sequentially eluted from the binding agent.

For example, in some embodiments, partitioning can be binary or based on degree/level of modifications. For example, all methylated fragments can be partitioned from unmethylated fragments using methyl-binding domain proteins (e.g., MethylMiner Methylated DNA Enrichment Kit (ThermoFisher Scientific)). Subsequently, additional partitioning may involve eluting fragments having different levels of methylation by adjusting the salt concentration in a solution with the methyl-binding domain and bound fragments. As salt concentration increases, fragments having greater methylation levels are eluted. In some instances, the final partitions are representative of nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MethylMiner Methylated DNA Enrichment Kit (ThermoFisher Scientific) various levels of methylation can be partitioned using sequential elutions. For example, a hypomethylated partition (e.g., no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, at least 300 mM, at least 400 mM, at least 500 mM, at least 600 mM, at least 700 mM, at least 800 mM, at least 900 mM, at least 1000 mM, or at least 2000 mM. After such methylated nucleic acids are eluted, magnetic separation is once again used to separate higher levels of methylated nucleic acids from those with lower level of methylation. The elution and magnetic separation steps can repeat themselves to create various partitions such as a hypomethylated partition (representative of no methylation), a methylated partition (representative of low level of methylation), and a hyper methylated partition (representative of high level of methylation).

In some methods, nucleic acids bound to an agent used for affinity separation are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent). The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition. For further details regarding portioning nucleic acid samples based on characteristics such as methylation, see WO2018/119452, which is incorporated herein by reference. In some embodiments, the nucleic acid molecules can be fractionated into different partitions based on the nucleic acid molecules that are bound to a specific protein or a fragment thereof and those that are not bound to that specific protein or fragment thereof.

Nucleic acid molecules can be fractionated based on DNA-protein binding. Protein-DNA complexes can be fractionated based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to fractionate the nucleic acid molecules based on protein bound regions. Examples of methods used to fractionate nucleic acid molecules based on protein bound regions include, but are not limited to, SDS-PAGE, chromatin-immuno-precipitation (ChIP), heparin chromatography, and asymmetrical field flow fractionation (AF4).

In some embodiments, partitioning of the nucleic acids is performed by contacting the nucleic acids with a methylation binding domain (“MBD”) of a methylation binding protein (“MBP”). MBD binds to 5-methylcytosine (5mC). MBD is coupled to paramagnetic beads, such as Dynabeads® M-280 Streptavidin via a biotin linker. Partitioning into fractions with different extents of methylation can be performed by eluting fractions by increasing the NaCl concentration.

An exemplary method for molecular tag identification of MBD-bead partitioned libraries through NGS is as follows:

Physical partitioning of an extracted DNA sample (e.g., extracted blood plasma DNA from a human sample) using a methyl-binding domain protein-bead purification kit, saving all elutions from process for downstream processing.

Parallel application of differential molecular tags and NGS-enabling adapter sequences to each partition. For example, the hypermethylated, residual methylation (‘wash’), and hypomethylated partitions are ligated with NGS-adapters with molecular tags.

Re-combining all molecular tagged partitions, and subsequent amplification using adapter-specific DNA primer sequences.

Enrichment/hybridization of re-combined and amplified total library, targeting genomic regions of interest (e.g., cancer-specific genetic variants and differentially methylated regions).

Re-amplification of the enriched total DNA library, appending a sample tag. Different samples are pooled and assayed in multiplex on an NGS instrument.

Bioinformatics analysis of NGS data, with the molecular tags being used to identify unique molecules, as well deconvolution of the sample into molecules that were differentially MBD-partitioned. This analysis can yield information on relative 5-methylcytosine for genomic regions, concurrent with standard genetic sequencing/variant detection.

Examples of MBPs contemplated herein include, but are not limited to:
(a) MeCP2 is a protein preferentially binding to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formylcytosine over unmodified cytosine (Iurlaro et al., Genome Biol. 14: R119 (2013)).
(d) Antibodies specific to one or more methylated nucleotide bases.

In general, elution is a function of number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nM to about 2500 mM NaCl. In one embodiment, the process results in three (3) partitions. Molecules are contacted with a solution at a first salt concentration and including a molecule including a methyl binding domain, which molecule can be attached to a capture moiety, such as streptavidin. At the first salt concentration a population of molecules will bind to the MBD and a population will remain unbound. The unbound population can be separated as a “hypomethylated” population. For example, a first partition representative of the hypomethylated form of DNA is that which remains unbound at a low salt concentration, e.g., 100 mM or 160 mM. A second partition representative of intermediate methylated DNA is eluted using an intermediate salt concentration, e.g., between 100 mM and 2000 mM concentration. This is also separated from the sample. A third partition representative of hypermethylated form of DNA is eluted using a high salt concentration, e.g., at least about 2000 mM.

The disclosure provides further methods for analyzing a population of nucleic acids in which at least some of the nucleic acids include one or more modified cytosine residues, such as 5-methylcytosine and any of the other modifications described previously. In these methods, after partitioning, the subsamples of nucleic acids are contacted with adapters including one or more cytosine residues modified at the 5C position, such as 5-methylcytosine. Preferably all cytosine residues in such adapters are also modified, or all such cytosines in a primer binding region of the adapters are modified. Adapters attach to both ends of nucleic acid molecules in the population. Preferably, the adapters include different tags of sufficient numbers that the number of combinations of tags results in a low probability e.g., 95, 99 or 99.9% of two nucleic acids with the same start and stop points receiving the same combination of tags. The primer binding sites in such adapters can be the same or different, but are preferably the same. After attachment of adapters, the nucleic acids are amplified from primers binding to the primer binding sites of the adapters. The amplified nucleic acids are split into first and second aliquots. The first aliquot is assayed for sequence data with or without further processing. The sequence data on molecules in the first aliquot is thus determined irrespective of the initial methylation state of the nucleic acid molecules. The nucleic acid molecules in the second aliquot are subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. The nucleic acids subjected to the procedure are then amplified with primers to the original primer binding sites of the adapters linked to nucleic acid. Only the nucleic acid molecules originally linked to adapters (as distinct from amplification products thereof) are now amplifiable because these nucleic acids retain cytosines in the primer binding sites of the adapters, whereas amplification products have lost the methylation of these cytosine residues, which have undergone conversion to uracils in the bisulfite treatment. Thus, only original molecules in the populations, at least some of which are methylated, undergo amplification. After amplification, these nucleic acids are subject to sequence analysis. Comparison of sequences determined from the first and second aliquots can indicate among other things, which cytosines in the nucleic acid population were subject to methylation.

Such an analysis can be performed using the following exemplary procedure. After partitioning, methylated DNA is linked to Y-shaped adapters at both ends including primer binding sites and tags. The cytosines in the adapters are modified at the 5 position (e.g., 5-methylated). The modification of the adapters serves to protect the primer binding sites in a subsequent conversion step (e.g., bisulfite treatment, TAP conversion, or any other conversion that does not affect the modified cytosine but affects unmodified cytosine). After attachment of adapters, the DNA molecules are amplified. The amplification product is split into two aliquots for sequencing with and without conversion. The aliquot not subjected to conversion can be subjected to sequence analysis with or without further processing. The other aliquot is subjected to a procedure that affects a first nucleobase in the DNA differently from a second nucleobase in the DNA, wherein the first nucleobase includes a cytosine modified at the 5 position, and the second nucleobase includes unmodified cytosine. This procedure may be bisulfite treatment or another procedure that converts unmodified cytosines to uracils. Only primer binding sites protected by modification of cytosines can support amplification when contacted with primers specific for original primer binding sites. Thus, only original molecules and not copies from the first amplification are subjected to further amplification. The further amplified molecules are then subjected to sequence analysis. Sequences can then be compared from the two aliquots. As in the separation scheme discussed above, nucleic acid tags in adapters are not used to distinguish between methylated and unmethylated DNA but to distinguish nucleic acid molecules within the same partition.

Subjecting the First Subsample to a Procedure that Affects a First Nucleobase in the DNA Differently from a Second Nucleobase in the DNA of the First Subsample

Methods disclosed herein comprise a step of subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (mC) and 5-hydroxymethylcytosine (hmC). Alternatively, the second nucleobase may comprise C and the first nucleobase may comprise one or more of mC and hmC. Other combinations are also possible, as indicated, e.g., in the Summary above and the following discussion, such as where one of the first and second nucleobases includes mC and the other includes hmC.

In some embodiments, the procedure that affects a first nucleobase in the DNA differently from a second nucleobase in the DNA of the first subsample includes bisulfite conversion. Treatment with bisulfite converts unmodified cytosine and certain modified cytosine nucleotides (e.g. 5-formyl cytosine (fC) or 5-carboxylcytosine (caC)) to uracil whereas other modified cytosines (e.g., 5-methylcytosine, 5-hydroxylmethylcystosine) are not converted. Thus, where bisulfite conversion is used, the first nucleobase includes one or more of unmodified cytosine, 5-formyl cytosine, 5-carboxylcytosine, or other cytosine forms affected by bisulfite, and the second nucleobase may comprise one or more of mC and hmC, such as mC and optionally hmC. Sequencing of bisulfite-treated DNA identifies positions that are read as cytosine as being mC or hmC positions. Meanwhile, positions that are read as T are identified as being T or a bisulfite-susceptible form of C, such as unmodified cytosine, 5-formyl cytosine, or 5-carboxylcytosine. Performing bisulfite conversion on a first subsample as described herein thus facilitates identifying positions containing mC or hmC using the sequence reads obtained from the first subsample. For an exemplary description of bisulfite conversion, see, e.g., Moss et al., Nat Commun. 2018; 9: 5068.

In some embodiments, the procedure that affects a first nucleobase in the DNA differently from a second nucleobase in the DNA of the first subsample includes oxidative bisulfite (Ox-BS) conversion. In some embodiments, the procedure that affects a first nucleobase in the DNA differently from a second nucleobase in the DNA of the first subsample includes Tet-assisted bisulfite (TAB) conversion. In some embodiments, the procedure that affects a first nucleobase in the DNA differently from a second nucleobase in the DNA of the first subsample includes Tet-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes chemical-assisted conversion with a substituted borane reducing agent, optionally wherein the substituted borane reducing agent is 2-picoline borane, borane pyridine, tert-butylamine borane, or ammonia borane. In some embodiments, the procedure that affects a first nucleobase in the DNA differently from a second nucleobase in the DNA of the first subsample includes APOBEC-coupled epigenetic (ACE) conversion.

In some embodiments, procedure that affects a first nucleobase in the DNA differently from a second nucleobase in the DNA of the first subsample includes enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-PGT can be used to convert 5mC and 5hmC into substrates that cannot be deaminated by a deaminase (e.g., APOBEC3A), and then a deaminase (e.g., APOBEC3A) can be used to deaminate unmodified cytosines converting them to uracils.

In some embodiments, the procedure that affects a first nucleobase in the DNA differently from a second nucleobase in the DNA of the first subsample includes separating DNA originally including the first nucleobase from DNA not originally including the first nucleobase.

In some embodiments, the first nucleobase is a modified or unmodified adenine, and the second nucleobase is a modified or unmodified adenine. In some embodiments, the modified adenine is N6-methyladenine (mA). In some embodiments, the modified adenine is one or more of N6-methyladenine (mA), N6-hydroxymethyladenine (hmA), or N6-formyladenine (fA).

Techniques including methylated DNA immunoprecipitation (MeDIP) can be used to separate DNA containing modified bases such as mA from other DNA. See, e.g., Kumar et al., Frontiers Genet. 2018; 9: 640; Greer et al., Cell 2015; 161: 868-878. An antibody specific for mA is described in Sun et al., Bioessays 2015; 37:1155-62. Antibodies for various modified nucleobases, such as forms of thymine/uracil including halogenated forms such as 5-bromouracil, are commercially available. Various modified bases can also be detected based on alterations in their base-pairing specificity. For example, hypoxanthine is a modified form of adenine that can result from deamination and is read in sequencing as a G. See, e.g., U.S. Pat. No. 8,486,630; Brown, Genomes, 2nd Ed., John Wiley & Sons, Inc., New York, N.Y., 2002, chapter 14, “Mutation, Repair, and Recombination.”

### Enrichinm/Capturinm Step, Amplification, Adaptors, Barcodes

In some embodiments, methods disclosed herein comprise a step of capturing one or more sets of target regions of DNA, such as cfDNA. Capture may be performed using any suitable approach known in the art. In some embodiments, capturing includes contacting the DNA to be captured with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. Where the first subsample undergoes a separation step (e.g., separating DNA originally including the first nucleobase (e.g., hmC) from DNA not originally including the first nucleobase, such as hmC-seal), capturing may be performed on any, any two, or all of the DNA originally including the first nucleobase (e.g., hmC), the DNA not originally including the first nucleobase, and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein includes capturing cfDNA obtained from a test subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules, and the cfDNA molecules corresponding to the sequence-variable target region set are captured at a greater capture yield in the captured set of cfDNA molecules than cfDNA molecules corresponding to the epigenetic target region set. For additional discussion of capturing steps, capture yields, and related aspects, see WO2020/160414, which is incorporated herein by reference for all purposes.

In some embodiments, a method described herein includes contacting cfDNA obtained from a test subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell).

In various embodiments, the methods further comprise sequencing the captured cfDNA, e.g., to different degrees of sequencing depth for the epigenetic and sequence-variable target region sets, consistent with the discussion herein. In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some such embodiments, the capturing step is performed with the probes for the sequence-variable target region set and the probes for the epigenetic target region set in the same vessel at the same time, e.g., the probes for the sequence-variable and epigenetic target region sets are in the same composition. This approach provides a relatively streamlined workflow. In some embodiments, the concentration of the probes for the sequence-variable target region set is greater that the concentration of the probes for the epigenetic target region set.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions including captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, the DNA is amplified. In some embodiments, amplification is performed before the capturing step. In some embodiments, amplification is performed after the capturing step.

In some embodiments, adapters are included in the DNA. This may be done concurrently with an amplification procedure, e.g., by providing the adapters in a 5′ portion of a primer, e.g., as described above. Alternatively, adapters can be added by other approaches, such as ligation.

In some embodiments, tags, which may be or include barcodes, are included in the DNA. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described above. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed in the “General Features of the Methods” section below, which can be combined to the extent practicable with any of the foregoing embodiments and the embodiments set forth in the introduction and summary section.

### Captured Set

In some embodiments, a captured set of DNA (e.g., cfDNA) is provided. With respect to the disclosed methods, the captured set of DNA may be provided, e.g., by performing a capturing step after a partitioning step as described herein. The captured set may comprise DNA corresponding to a sequence-variable target region set, an epigenetic target region set, or a combination thereof. In some embodiments the quantity of captured sequence-variable target region DNA is greater than the quantity of the captured epigenetic target region DNA, when normalized for the difference in the size of the targeted regions (footprint size).

Alternatively, first and second captured sets may be provided, including, respectively, DNA corresponding to a sequence-variable target region set and DNA corresponding to an epigenetic target region set. The first and second captured sets may be combined to provide a combined captured set.

In some embodiments in which a captured set including DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80 to 90-fold greater concentration, a 90- to 100-fold greater concentration, a 10- to 20-fold greater concentration, a 10- to 40-fold greater concentration, a 10- to 50-fold greater concentration, a 10 to 70-fold greater concentration, or a 10- to 100-fold greater concentration. The degree of difference in concentrations accounts for normalization for the footprint sizes of the target regions, as discussed in the definition section.

### Epigenetic Target Region Set

The epigenetic target region set may comprise one or more types of target regions likely to differentiate DNA from neoplastic (e.g., tumor or cancer) cells and from healthy cells, e.g., non-neoplastic circulating cells. Exemplary types of such regions are discussed in detail herein. The epigenetic target region set may also comprise one or more control regions, e.g., as described herein. In some embodiments, the epigenetic target region set has a footprint of at least 100 kb, e.g., at least 200 kb, at least 300 kb, or at least 400 kb. In some embodiments, the epigenetic target region set has a footprint in the range of 100-1000 kb, e.g., 100-200 kb, 200-300 kb, 300-400 kb, 400-500 kb, 500-600 kb, 600-700 kb, 700-800 kb, 800-900 kb, and 900-1,000 kb.

### Hypermethylation Variable Target Regions

In some embodiments, the epigenetic target region set includes one or more hypermethylation variable target regions. In general, hypermethylation variable target regions refer to regions where an increase in the level of observed methylation, e.g., in a cfDNA sample, indicates an increased likelihood that a sample (e.g., of cfDNA) contains DNA produced by neoplastic cells, such as tumor or cancer cells. For example, hypermethylation of promoters of tumor suppressor genes has been observed repeatedly. See, e.g., Kang et al., Genome Biol. 18:53 (2017) and references cited therein. In an example, hypermethylation variable target regions can include regions that do not necessarily differ in methylation in cancerous tissue relative to DNA from healthy tissue of the same type, but do differ in methylation (e.g., have more methylation) relative to cfDNA that is typical in healthy subjects. Where, for example, the presence of a cancer results in increased cell death such as apoptosis of cells of the tissue type corresponding to the cancer, such a cancer can be detected at least in part using such hypermethylation variable target regions. In some embodiments, hypermethylation variable target regions include one or more genomic regions, where the cfDNA molecules in those regions do not differ in methylation state in cancer subjects relative to cfDNA from healthy subjects, but the presence/increased quantity of hypermethylated cfDNA in those regions is indicative of a particular tissue type (e.g., cancer origin) and is presented as cfDNA with increased apoptosis (e.g. tumor shedding) into circulation.

Hypermethylation target regions may be obtained, e.g., from the Cancer Genome Atlas. Kang et al., Genome Biology 18:53 (2017), describe construction of a probabilistic method called CancerLocator using hypermethylation target regions from breast, colon, kidney, liver, and lung. In some embodiments, the hypermethylation target regions can be specific to one or more types of cancer. Accordingly, in some embodiments, the hypermethylation target regions include one, two, three, four, or five subsets of hypermethylation target regions that collectively show hypermethylation in one, two, three, four, or five of breast, colon, kidney, liver, and lung cancers.

In some embodiments, the probes for the epigenetic target region set comprise probes specific for one or more hypermethylation variable target regions. The hypermethylation variable target regions may be any of those set forth above. For example, in some embodiments, the probes specific for hypermethylation variable target regions comprise probes specific for a plurality of loci listed in Table 1, e.g., at least 10%, 20%, 30%, 40%, 50%, 60%, 70%, 80%, 90%, or 100% of the loci listed in Table 1. In some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 1 or Table 2, e.g., at least 10%, 20%, 30%, 40%, 50%, 60%, 70%, 80%, 90%, or 100% of the loci listed in Table 1 or Table 2. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

### Hypomethylation Variable Target Regions

Global hypomethylation is a commonly observed phenomenon in various cancers. See, e.g., Hon et al., Genome Res. 22:246-258 (2012) (breast cancer); Ehrlich, Epigenomics 1:239-259 (2009) (review article noting observations of hypomethylation in colon, ovarian, prostate, leukemia, hepatocellular, and cervical cancers). For example, regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells. Accordingly, in some embodiments, the epigenetic target region set includes hypomethylation variable target regions, where a decrease in the level of observed methylation indicates an increased likelihood that a sample (e.g., of cfDNA) contains DNA produced by neoplastic cells, such as tumor or cancer cells. In an example, hypomethylation variable target regions can include regions that do not necessarily differ in methylation state in cancerous tissue relative to DNA from healthy tissue of the same type, but do differ in methylation (e.g., are less methylated) relative to cfDNA that is typical in healthy subjects. Where, for example, the presence of a cancer results in increased cell death such as apoptosis of cells of the tissue type corresponding to the cancer, such a cancer can be detected at least in part using such hypomethylation variable target regions. In some embodiments, hypomethylation variable target regions include one or more genomic regions, where the cfDNA molecules in those regions do not differ in methylation state in cancer subjects relative to cfDNA from healthy subjects, but the presence/increased quantity of hypomethylated cfDNA in those regions is indicative of a particular tissue type (e.g., cancer origin) and is presented as cfDNA with increased apoptosis (e.g. tumor shedding) into circulation.

In some embodiments, hypomethylation variable target regions include repeated elements and/or intergenic regions. In some embodiments, repeated elements include one, two, three, four, or five of LINE1 elements, Alu elements, centromeric tandem repeats, pericentromeric tandem repeats, and/or satellite DNA.

Exemplary specific genomic regions that show cancer-associated hypomethylation include nucleotides 8403565-8953708 and 151104701-151106035 of human chromosome 1. In some embodiments, the hypomethylation variable target regions overlap or comprise one or both of these regions.

In some embodiments, the probes for the epigenetic target region set comprise probes specific for one or more hypomethylation variable target regions. The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or including nucleotides 8403565-8953708 and/or 151104701-151106035 of human chromosome

Probes for detecting the panel of regions can include those for detecting genomic regions of interest (hotspot regions) as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. Subjects

In some embodiments, the DNA (e.g., cfDNA) is obtained from a subject having a cancer. In some embodiments, the DNA (e.g., cfDNA) is obtained from a subject suspected of having a cancer. In some embodiments, the DNA (e.g., cfDNA) is obtained from a subject having a tumor. In some embodiments, the DNA (e.g., cfDNA) is obtained from a subject suspected of having a tumor. In some embodiments, the DNA (e.g., cfDNA) is obtained from a subject having neoplasia. In some embodiments, the DNA (e.g., cfDNA) is obtained from a subject suspected of having neoplasia. In some embodiments, the DNA (e.g., cfDNA) is obtained from a subject in remission from a tumor, cancer, or neoplasia (e.g., following chemotherapy, surgical resection, radiation, or a combination thereof). In any of the foregoing embodiments, the cancer, tumor, or neoplasia or suspected cancer, tumor, or neoplasia may be of the lung, colon, rectum, kidney, breast, prostate, or liver. In some embodiments, the cancer, tumor, or neoplasia or suspected cancer, tumor, or neoplasia is of the lung. In some embodiments, the cancer, tumor, or neoplasia or suspected cancer, tumor, or neoplasia is of the colon or rectum. In some embodiments, the cancer, tumor, or neoplasia or suspected cancer, tumor, or neoplasia is of the breast. In some embodiments, the cancer, tumor, or neoplasia or suspected cancer, tumor, or neoplasia is of the prostate. In any of the foregoing embodiments, the subject may be a human subject.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb.

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

### Compositions Including Captured DNA

Provided herein is a combination including first and second populations of captured DNA. The first population may comprise or be derived from DNA with a cytosine modification in a greater proportion than the second population. The first population may comprise a form of a first nucleobase originally present in the DNA with altered base pairing specificity and a second nucleobase without altered base pairing specificity, wherein the form of the first nucleobase originally present in the DNA prior to alteration of base pairing specificity is a modified or unmodified nucleobase, the second nucleobase is a modified or unmodified nucleobase different from the first nucleobase, and the form of the first nucleobase originally present in the DNA prior to alteration of base pairing specificity and the second nucleobase have the same base pairing specificity. The second population does not comprise the form of the first nucleobase originally present in the DNA with altered base pairing specificity. In some embodiments, the cytosine modification is cytosine methylation. In some embodiments, the first nucleobase is a modified or unmodified cytosine and the second nucleobase is a modified or unmodified cytosine. The first and second nucleobase may be any of those discussed herein in the Summary or with respect to subjecting the first subsample to a procedure that affects a first nucleobase in the DNA differently from a second nucleobase in the DNA of the first subsample.

In some embodiments, the first population includes a sequence tag selected from a first set of one or more sequence tags and the second population includes a sequence tag selected from a second set of one or more sequence tags, and the second set of sequence tags is different from the first set of sequence tags. The sequence tags may comprise barcodes.

In some embodiments, the first population includes protected hmC, such as glucosylated hmC. In some embodiments, the first population was subjected to any of the conversion procedures discussed herein, such as bisulfite conversion, Ox-BS conversion, TAB conversion, ACE conversion, TAP conversion, TAPSO conversion, or CAP conversion. In some embodiments, the first population was subjected to protection of hmC followed by deamination of mC and/or C. In some embodiments of the combination, the first population includes or was derived from DNA with a cytosine modification in a greater proportion than the second population and the first population includes first and second subpopulations, and the first nucleobase is a modified or unmodified nucleobase, the second nucleobase is a modified or unmodified nucleobase different from the first nucleobase, and the first nucleobase and the second nucleobase have the same base pairing specificity. In some embodiments, the second population does not comprise the first nucleobase. In some embodiments, the first nucleobase is a modified or unmodified cytosine, and the second nucleobase is a modified or unmodified cytosine, optionally wherein the modified cytosine is mC or hmC. In some embodiments, the first nucleobase is a modified or unmodified adenine, and the second nucleobase is a modified or unmodified adenine, optionally wherein the modified adenine is mA.

In some embodiments, the first nucleobase (e.g., a modified cytosine) is biotinylated. In some embodiments, the first nucleobase (e.g., a modified cytosine) is a product of a Huisgen cycloaddition to (3-6-azide-glucosyl-5-hydroxymethylcytosine that includes an affinity label (e.g., biotin).

In any of the combinations described herein, the captured DNA may comprise cfDNA. The captured DNA may have any of the features described herein concerning captured sets, including, e.g., a greater concentration of the DNA corresponding to the sequence-variable target region set (normalized for footprint size as discussed above) than of the DNA corresponding to the epigenetic target region set. In some embodiments, the DNA of the captured set includes sequence tags, which may be added to the DNA as described herein. In general, the inclusion of sequence tags results in the DNA molecules differing from their naturally occurring, untagged form.

The combination may further comprise a probe set described herein or sequencing primers, each of which may differ from naturally occurring nucleic acid molecules. For example, a probe set described herein may comprise a capture moiety, and sequencing primers may comprise a non-naturally occurring label.

### Computer Systems, Processing of Real World Evidence (RWE)

Methods of the present disclosure can be implemented using, or with the aid of, computer systems. For example, such methods may comprise: partitioning the sample into a plurality of subsamples, including a first subsample and a second subsample, wherein the first subsample includes DNA with a cytosine modification in a greater proportion than the second subsample; subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity; and sequencing DNA in the first subsample and DNA in the second subsample in a manner that distinguishes the first nucleobase from the second nucleobase in the DNA of the first subsample.

In an aspect, the present disclosure provides a non-transitory computer-readable medium including computer-executable instructions which, when executed by at least one electronic processor, perform at least a portion of a method including: collecting cfDNA from a test subject; capturing a plurality of sets of target regions from the cfDNA, wherein the plurality of target region sets includes a sequence-variable target region set and an epigenetic target region set, whereby a captured set of cfDNA molecules is produced; sequencing the captured cfDNA molecules, wherein the captured cfDNA molecules of the sequence-variable target region set are sequenced to a greater depth of sequencing than the captured cfDNA molecules of the epigenetic target region set; obtaining a plurality of sequence reads generated by a nucleic acid sequencer from sequencing the captured cfDNA molecules; mapping the plurality of sequence reads to one or more reference sequences to generate mapped sequence reads; and processing the mapped sequence reads corresponding to the sequence-variable target region set and to the epigenetic target region set to determine the likelihood that the subject has cancer.

The code can be pre-compiled and configured for use with a machine with a processer adapted to execute the code or can be compiled during runtime. The code can be supplied in a programming language that can be selected to enable the code to execute in a pre-compiled or as-compiled fashion.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7th Ed. (2016), Elmasri, Fundamentals of Database Systems, Addison Wesley, 6th Ed. (2010), Coronel, Database Systems: Design, Implementation, & Management, Cengage Learning, 11th Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety. Further information is found in PCT Pub. No. US2022032250 and U.S. application Ser. No. 17/832,498.

Described herein is a method to generate an integrated data repository that includes multiple types of healthcare data, according to one or more implementations. The architecture may include a data integration and analysis system. The data integration and analysis system may obtain data from a number of data sources and integrate the data from the data sources into an integrated data repository. For example, the data integration and analysis system may obtain data from a electronic medical record, including for example, patient information, health insurance claims, lifestyle data, repository. In various examples, the data integration and analysis system and the electronic medical record, including for example, patient information, health insurance claims, lifestyle data, repository may be created and maintained by different entities. In one or more additional examples, the data integration and analysis system and the electronic medical record, including for example, patient information, health insurance claims, lifestyle data, repository may be created and maintained by the same entity.

The data integration and analysis system may be implemented by one or more computing devices. The one or more computing devices may include one or more server computing devices, one or more desktop computing devices, one or more laptop computing devices, one or more tablet computing devices, one or more mobile computing devices, or combinations thereof. In certain implementations, at least a portion of the one or more computing devices may be implemented in a distributed computing environment. For example, at least a portion of the one or more computing devices may be implemented in a cloud computing architecture. In scenarios where the computing systems used to implement the data integration and analysis system are configured in a distributed computing architecture, processing operations may be performed concurrently by multiple virtual machines. In various examples, the data integration and analysis system may implement multithreading techniques. The implementation of a distributed computing architecture and multithreading techniques cause the data integration and analysis system to utilize fewer computing resources in relation to computing architectures that do not implement these techniques.

The electronic medical record, including for example, patient information, health insurance claims, lifestyle data, repository may store information obtained from one or more health insurance companies that corresponds to insurance claims made by subscribers of the one or more health insurance companies. The electronic medical record, including for example, patient information, health insurance claims, lifestyle data, repository may be arranged (e.g., sorted) by patient identifier. The patient identifier may be based on the patient's first name, last name, date of birth, social security number, address, employer, and the like. The data stored by the electronic medical record, including for example, patient information, health insurance claims, lifestyle data, repository may include structured data that is arranged in one or more data tables. The one or more data tables storing the structured data may include a number of rows and a number of columns that indicate information about health insurance claims made by subscribers of one or more health insurance companies in relation to procedures and/or treatments received by the subscribers from healthcare providers. At least a portion of the rows and columns of the data tables stored by the electronic medical record, including for example, patient information, health insurance claims, lifestyle data, repository may include health insurance codes that may indicate diagnoses of biological conditions, and treatments and/or procedures obtained by subscribers of the one or more health insurance companies. In various examples, the health insurance codes may also indicate diagnostic procedures obtained by individuals that are related to one or more biological conditions that may be present in the individuals. In one or more examples, a diagnostic procedure may provide information used in the detection of the presence of a biological condition. A diagnostic procedure may also provide information used to determine a progression of a biological condition. In one or more illustrative examples, a diagnostic procedure may include one or more imaging procedures, one or more assays, one or more laboratory procedures, one or more combinations thereof, and the like.

The data integration and analysis system may also obtain information from a molecular data repository. The molecular data repository may store data of a number of individuals related to genomic information, genetic information, metabolomic information, transcriptomic information, fragmentomic information, immune receptor information, methylation information, epigenomic information, and/or proteomic information. In one or more examples, the data integration and analysis system and the molecular data repository may be created and maintained by different entities. In one or more additional examples, the data integration and analysis system and the molecular data repository may be created and maintained by a same entity.

The genomic information may indicate one or more mutations corresponding to genes of the individuals. A mutation to a gene of individuals may correspond to differences between a sequence of nucleic acids of the individuals and one or more reference genomes. The reference genome may include a known reference genome, such as hg19. In various examples, a mutation of a gene of an individual may correspond to a difference in a germline gene of an individual in relation to the reference genome. In one or more additional examples, the reference genome may include a germline genome of an individual. In one or more further examples, a mutation to a gene of an individual may include a somatic mutation. Mutations to genes of individuals may be related to insertions, deletions, single nucleotide variants, loss of heterozygosity, duplication, amplification, translocation, fusion genes, or one or more combinations thereof.

In one or more illustrative examples, genomic information stored by the molecular data repository may include genomic profiles of tumor cells present within individuals. In these situations, the genomic information may be derived from an analysis of genetic material, such as deoxyribonucleic acid (DNA) and/or ribonucleic acid (RNA) from a sample, including, but not limited to, a tissue sample or tumor biopsy, circulating tumor cells (CTCs), exosomes or efferosomes, or from circulating nucleic acids (e.g., cell-free DNA) found in blood samples of individuals that is present due to the degradation of tumor cells present in the individuals. In one or more examples, the genomic information of tumor cells of individuals may correspond to one or more target regions. One or more mutations present with respect to the one or more target regions may indicate the presence of tumor cells in individuals. The genomic information stored by the molecular data repository may be generated in relation to an assay or other diagnostic test that may determine one or more mutations with respect to one or more target regions of the reference genome.

In one or more additional examples, the data integration and analysis system may obtain information from one or more additional data repositories. The one or more additional data repositories may store data related to electronic medical records of individuals for which data is present in at least one of the electronic medical record, including for example, patient information, health insurance claims, lifestyle data, repository or the molecular data repository. Further, the one or more additional data repositories may store data related to pathology reports of individuals for which data is present in at least one of the electronic medical record, including for example, patient information, health insurance claims, lifestyle data, repository or the molecular data repository. In various examples, the one or more additional data repositories may store data related to biological conditions and/or treatments for biological conditions. In one or more examples, the data integration and analysis system and at least a portion of the one or more additional data repositories may be created and maintained by different entities. In one or more further examples, the data integration and analysis system and at least a portion of the one or more additional data repositories may be created and maintained by a same entity.

In one or more further implementations, the data integration and analysis system may obtain information from one or more reference information data repositories. The one or more reference information data repositories may store information that includes definitions, standards, protocols, vocabularies, one or more combinations thereof, and the like. In various examples, the information stored by the one or more reference information data repositories may correspond to biological conditions and/or treatments for biological conditions. In one or more illustrative examples, the one or more reference information data repositories may include RxNorm. (RxNorm provides normalized names for clinical drugs and links its names to many of the drug vocabularies used in pharmacy management and drug interaction software.) In one or more examples, the data integration and analysis system and at least a portion of the one or more reference information data repositories may be created and maintained by different entities. In one or more further examples, the data integration and analysis system and at least a portion of the one or more reference information data repositories may be created and maintained by a same entity.

The data integration and analysis system may obtain data from at least one of the electronic medical record, including for example, patient information, health insurance claims, lifestyle data, repository, the molecular data repository, the one or more additional data repositories, or the reference information data repositories via one or more communication networks accessible to the data integration and analysis system and accessible to at least one of the electronic medical record, including for example, patient information, health insurance claims, lifestyle data, repository, the molecular data repository, the one or more additional data repositories, or the reference information data repositories. The data integration and analysis system may also obtain data from at least one of the electronic medical record, including for example, patient information, health insurance claims, lifestyle data, repository, the molecular data repository, the one or more additional data repositories, or the reference information data repositories via one or more secure communication channels. In addition, the data integration and analysis system may obtain data from at least one of the electronic medical record, including for example, patient information, health insurance claims, lifestyle data, repository, the molecular data repository, the one or more additional data repositories, or the reference information data repositories via one or more calls of an application programming interface (API).

The data integration and analysis system may include a data integration system. The data integration system may obtain data from the electronic medical record, including for example, patient information, health insurance claims, lifestyle data, repository and the molecular data repository to generate the integrated data repository. The data integration system may also obtain data from the one or more additional data repositories to generate the integrated data repository. In various examples, the data integration system may implement one or more natural language processing techniques to integrate data from the one or more additional data repositories into the integrated data repository.

In one or more examples, the data integration system may generate one or more tokens to identify individuals that have data stored in the electronic medical record, including for example, patient information, health insurance claims, lifestyle data, repository and that have data stored in the molecular data repository. In various examples, the data integration system may generate one or more tokens by implementing one or more hash functions. The data integration system may implement the one or more hash functions to generate the one or more tokens based on information stored by at least one of the electronic medical record, including for example, patient information, health insurance claims, lifestyle data, repository or the molecular data repository. For example, the information used by the data integration system to generate individual tokens by implementing a hash function may include at least one of an identifiers of respective individuals, date of birth of the respective individuals, a postal code of the respective individuals, date of birth of the respective individuals, or a gender of the respective individuals. In one or more illustrative examples, the identifiers of the respective individuals may include a combination of at least a portion of a first name of the respective individuals and at least a portion of the last name of the respective individuals. Tokens generated using data from different data repositories may correspond to the same or similar information or the same or similar type stored by the different data repositories. To illustrate, tokens may be generated using a portion of names of individuals, date of birth, at least a portion of a postal code, and gender obtained from the electronic medical record, including for example, patient information, health insurance claims, lifestyle data, repository and the molecular data repository.

The data integration system may integrate data from a number of different data sources by analyzing tokens generated by implementing one or more hash functions using data obtained from the number of different data sources. For example, the data integration system may obtain one or more first tokens generated from data stored by the electronic medical record, including for example, patient information, health insurance claims, lifestyle data, repository and one or more second tokens generated from data stored by the molecular data repository. The data integration system may analyze the one or more first tokens with respect to the one or more second tokens to determine individual first tokens that correspond to individual second tokens. In one or more illustrative examples, the data integration system may identify individual first tokens that match individual second tokens. A first token may match a second token when the data of the first token has at least a threshold amount of similarity with respect to the data of the second token. In one or more examples, a first token may match a second token when the data of the first token is the same as the data of the second token. To illustrate, a first token may match a second token when an alphanumeric string of the first token is the same as an alphanumeric string of the second token.

By determining a first token generated using data stored by the electronic medical record, including for example, patient information, health insurance claims, lifestyle data, repository that corresponds to a second token generated using data stored by the molecular data repository, the data integration system may identify an individual having data that is stored in both the electronic medical record, including for example, patient information, health insurance claims, lifestyle data, repository and in the molecular data repository. In this way, the data integration system may obtain data from the electronic medical record, including for example, patient information, health insurance claims, lifestyle data, repository from a number of individuals and data from the molecular data repository from the same number of individuals and store the electronic medical record, including for example, patient information, health insurance claims, lifestyle data, and the molecular data for the number of individuals in the integrated data repository.

The data integration system may also integrate data stored by the one or more additional data repositories with data from the electronic medical record, including for example, patient information, health insurance claims, lifestyle data, repository and the molecular data repository to generate the integrated data repository. To illustrate, the data integration system may obtain one or more thirds of tokens generated from data stored by an additional data repository, such as a data repository storing data corresponding to pathology reports. The data integration system may analyze the one or more third tokens with respect to the first tokens generated using information stored by the electronic medical record, including for example, patient information, health insurance claims, lifestyle data, repository and the second tokens generated using information stored by the molecular data repository to determine respective third tokens that correspond to individuals first tokens and individual second tokens. In one or more illustrative examples, the data integration system may identify third tokens generated using one or more hash functions and a common set of information obtained from the electronic medical record, including for example, patient information, health insurance claims, lifestyle data, repository, the molecular data repository, and the additional data repository.

By determining a third token generated using data stored by an additional data repository that corresponds to a first token generated using data stored by the electronic medical record, including for example, patient information, health insurance claims, lifestyle data, repository and a second token generated using data stored by the molecular data repository, the data integration system may identify an individual having data that is stored in the electronic medical record, including for example, patient information, health insurance claims, lifestyle data, repository, the molecular data repository, and in an additional data repository. In this way, the data integration system may obtain data from the electronic medical record, including for example, patient information, health insurance claims, lifestyle data, repository from a number of individuals and data from the molecular data repository and an additional data repository from the same number of individuals and store the electronic medical record, including for example, patient information, health insurance claims, lifestyle data, the molecular data, and the additional data for the number of individuals in the integrated data repository.

The data stored by the integrated data repository for the number of individuals may be accessible using respective identifiers of individuals. The data integration system may implement a number of techniques as part of a de-identification process with respect to storing and retrieving information of individuals in the integrated data repository. The identifiers of individuals may correspond to keys that are generated using at least one hash function. The identifiers of the individuals may also be generated by implementing one or more salting processes with respect to the keys generated using the at least one hash function. the tokens generated using one or more hash functions and a common set of information obtained from the electronic medical record, including for example, patient information, health insurance claims, lifestyle data, repository, the molecular data repository, and/or the additional data repository. In one or more illustrative examples, the identifiers generated by the data integration system to access information for respective individuals that is stored by the integrated data repository may be unique for each individual. In one or more examples, the identifiers of the individuals may be generated using at least a portion of the information used to generate the tokens related to the individuals. In one or more additional examples, the identifiers of the individuals may be generated using different information from the information used to generate the tokens related to the individuals.

The data integration system may also generate the integrated data repository from a number of different combinations of data repositories in a similar manner. For example, the data integration system may obtain tokens generated from information stored by the electronic medical record, including for example, patient information, health insurance claims, lifestyle data, repository and additional tokens generated from information stored by one or more additional data stores. The data integration system may determine individual tokens generated from information stored by the electronic medical record, including for example, patient information, health insurance claims, lifestyle data, repository that correspond to individual additional tokens generated from information stored by the one or more additional data repositories. By determining tokens generated using data stored by the electronic medical record, including for example, patient information, health insurance claims, lifestyle data, repository that correspond to additional tokens generated using data stored by an additional data repository, the data integration system may identify individuals having data that is stored in both the electronic medical record, including for example, patient information, health insurance claims, lifestyle data, repository and in the additional data repository. In this way, the data integration system may obtain data from the electronic medical record, including for example, patient information, health insurance claims, lifestyle data, repository from a number of individuals and data from the additional data repository from the same number of individuals and store the electronic medical record, including for example, patient information, health insurance claims, lifestyle data, and the additional data for the number of individuals in the integrated data repository. The electronic medical record, including for example, patient information, health insurance claims, lifestyle data, and the additional data stored by the integrated data repository for the number of individuals may be accessible using respective identifiers of individuals.

In one or more further examples, the data integration system may obtain tokens generated from information stored by the molecular data repository and tokens generated from information stored by one or more additional data stores. The data integration system may determine individual tokens generated from information stored by the molecular data repository that correspond to individual additional tokens generated from information stored by the one or more additional data repositories. By determining tokens generated using data stored by the molecular data repository that correspond to additional tokens generated using data stored by an additional data repository, the data integration system may identify individuals having data that is stored in both the molecular data repository and in the additional data repository. In this way, the data integration system may obtain data from the molecular data repository from a number of individuals and data from the additional data repository from the same number of individuals and store the molecular data and the additional data for the number of individuals in the integrated data repository. The molecular data and the additional data stored by the integrated data repository for the number of individuals may be accessible using respective identifiers of individuals.

The data stored by the integrated data repository may be stored according to one or more regulatory frameworks that protect the privacy and ensure the security of medical records, health information, and insurance information of individuals. For example, data may be stored by the integrated data repository in accordance with one or more governmental regulatory frameworks directed to protecting personal information, such as the Health Insurance Portability and Accountability Act (HIPAA) and/or the General Data Protection Regulation (GDPR). The integrated data repository also stores data in an anonymized and de-identified manner to ensure protection of the privacy of individuals that have data stored by the integrated data repository. To further ensure the privacy of individuals that have data stored by the integrated data repository, the data integration system may re-generate the integrated data repository periodically. For example, the data integration system may create the integrated data repository once per quarter. In one or more additional examples, the data integration system may generate the integrated data repository on a monthly basis, on a weekly basis, or once every two weeks. By re-generating the integrated data repository on a periodic basis and not simply refreshing the integrated data repository when new data is available, the integrated data repository enhances privacy protection with respect to data stored by the integrated data repository. That is, in situations where data repositories are refreshed simply with new data, it may be possible to more easily track individuals associated with data that has been newly added to a data repository because the number of new individuals added at a given time is typically smaller than an existing number of individuals that already have data stored by the data repository.

In various examples, data stored by the integrated data repository may be accessed via a database management system. In addition, the integrated data repository may store data according to one or more database models. In one or more examples, the integrated data repository may store data according to one or more relational database technologies. For example, the integrated data repository may store data according to a relational database model. In one or more additional examples, the integrated data repository may store data according to an object-oriented database model. In one or more further examples, the integrated data repository may store data according to an extensible markup language (XML) database model. In additional examples, the integrated data repository may store data according to a structured query language (SQL) database model. In still further examples, the integrated data repository may store data according to an image database model.

The data integration system may generate the integrated data repository by generating a number of data tables and creating links between the data tables. The links may indicate logical couplings between the data tables. The data integration system may generate the data tables by extracting specified sets of data from the information obtained from the data repositories and storing the data in rows and columns of respective data tables. In various examples, the logical couplings between data tables may include at least one of a one-to-one link where a row of information in one data table corresponds to a row of information in another data table, a one-to-many link where a row of information in one data table corresponds to multiple rows of information in another data table, or a many-to-many link where multiple rows of information of one data table correspond to multiple rows of information in another data table.

The number of data tables may be arranged according to a data repository schema. In the illustrative example of the data repository schema includes a first data table, a second data table, a third data table, a fourth data table, and a fifth data table. Although the illustrative example of includes five data tables, in additional implementations, the data repository schema may include more data tables or fewer data tables. The data repository schema may also include links between the data tables. The links between the data tables may indicate that information retrieved from one of the data tables results in additional information stored by one or more additional data tables to be retrieved. Additionally, not all the data tables may be linked to each of the other data tables. In the illustrative example of the first data table is logically coupled to the second data table by a first link and the first data table is logically coupled to the fourth data table by a second link. In addition, the second data table is logically coupled to the third data table via a third link and the fourth data table is logically coupled to the fifth data table via a fourth link. Further, the third data table is logically coupled to the fifth data table via a fifth link.

In various examples, as data tables are added to and/or removed from the data repository schema, additional links between data tables may be added to or removed from the data repository schema. In one or more illustrative examples, the integrated data repository may store data tables according to the data repository schema for at least a portion of the individuals for which the data integration system obtained information from a combination of at least two of the electronic medical record, including for example, patient information, health insurance claims, lifestyle data, repository, the molecular data repository, the one or more additional data repositories, and the one or more reference information data repositories. As a result, the integrated data repository may store respective instances of the data tables according to the data repository schema for thousands, tens of thousands, up to hundreds of thousands or more individuals.

The data integration and analysis system may also include a data pipeline system. The data pipeline system may include a number of algorithms, software code, scripts, macros, or other bundles of computer-executable instructions that process information stored by the integrated data repository to generate additional datasets. The additional datasets may include information obtained from one or more of the data tables. The additional datasets may also include information that is derived from data obtained from one or more of the data tables. The components of the data pipeline system implemented to generate a first additional dataset may be different from the components of the data pipeline system used to generate a second additional dataset.

In one or more examples, the data pipeline system may generate a dataset that indicates pharmacy treatments received by a number of individuals. In one or more illustrative examples, the data pipeline system may analyze information stored in at least one of the data tables to determine health insurance codes corresponding to pharmaceutical treatments received by a number of individuals. The data pipeline system may analyze the health insurance codes corresponding to pharmaceutical treatments with respect to a library of data that indicates specified pharmaceutical treatments that correspond to one or more health insurance codes to determine names of pharmaceutical treatments that have been received by the individuals. In one or more additional examples, the data pipeline system may analyze information stored by the integrated data repository to determine medical procedures received by a number of individuals. To illustrate, the data pipeline system may analyze information stored by one of the data tables to determine treatments received by individuals via at least one injection or intravenously. In one or more further examples, the data pipeline system may analyze information stored by the integrated data repository to determine episodes of care for individuals, lines of therapy received by individuals, progression of a biological condition, or time to next treatment. In various examples, the datasets generated by the data pipeline system may be different for different biological conditions. For example, the data pipeline system may generate a first number of datasets with respect to a first type of cancer, such as lung cancer, and a second number of datasets with respect to a second type of cancer, such as colorectal cancer.

The data pipeline system may also determine one or more confidence levels to assign to information associated with individuals having data stored by the integrated data repository. The respective confidence levels may correspond to different measures of accuracy for information associated with individuals having data stored by the integrated data repository. The information associated with the respective confidence levels may correspond to one or more characteristics of individuals derived from data stored by the integrated data repository. Values of confidence levels for the one or more characteristics may be generated by the data pipeline system in conjunction with generating one or more datasets from the integrated data repository. In one or more examples, a first confidence level may correspond to a first range of measures of accuracy, a second confidence level may correspond to a second range of measures of accuracy, and a third confidence level may correspond to a third range of measures of accuracy. In one or more additional examples, the second range of measures of accuracy may include values that are less values of the first range of measures of accuracy and the third range of measures of accuracy may include values that are less than values of the second range of measures of accuracy. In one or more illustrative examples, information corresponding to the first confidence level may be referred to as Gold standard information, information corresponding to the second confidence level may be referred to as Silver standard information, and information corresponding to the third confidence level may be referred to as Bronze standard information.

The data pipeline system may determine values for the confidence levels of characteristics of individuals based on a number of factors. For example, a respective set of information may be used to determine characteristics of individuals. The data pipeline system may determine the confidence levels of characteristics of individuals based on an amount of completeness of the respective set of information used to determine a characteristic for an individual. In situations where one or more pieces of information are missing from the set of information associated with a first number of individuals, the confidence levels for a characteristic may be lower than for a second number of individuals where information is not missing from the set of information. In one or more examples, an amount of missing information may be used by the data pipeline system to determine confidence levels of characteristics of individuals. To illustrate, a greater amount of missing information used to determine a characteristic of an individual may cause confidence levels for the characteristic to be lower than in situations where the amount of missing information used to determine the characteristic is lower. Further, different types of information may correspond to various confidence levels for a characteristic. In one or more examples, the presence of a first piece of information used to determine a characteristic of an individual may result in confidence levels for the characteristic being higher than the presence of a second piece of information used to determine the characteristic.

In one or more illustrative examples, the data pipeline system may determine a number of individuals included in a cohort with a primary diagnosis of lung cancer (or other biological condition). The data pipeline system may determine confidence levels for respective individuals with respect to being classified as having a primary diagnosis of lung cancer. The data pipeline system may use information from a number of columns included in the data tables to determine a confidence level for the inclusion of individuals within a lung cancer cohort. The number of columns may include health insurance codes related to diagnosis of biological conditions and/or treatments of biological conditions. Additionally, the number of columns may correspond to dates of diagnosis and/or treatment for biological conditions. The data pipeline system may determine that a confidence level of an individual being characterized as being part of the lung cancer cohort is higher in scenarios where information is available for each of the number of columns or at least a threshold number of columns than in instances where information is available for less than a threshold number of columns. Further, the data pipeline system may determine confidence levels for individuals included in a lung cancer cohort based on the type of information and availability of information associated with one or more columns. To illustrate, in situations where one or more diagnosis codes are present in relation to one or more periods of time for a group of individuals and one or more treatment codes are absent, the data pipeline system may determine that the confidence level of including the group of individuals in the lung cancer cohort is greater than in situations where at least one of the diagnosis codes is absent and the treatment codes used to determine whether individuals are included in the lung cancer cohort are present.

The data integration and analysis system may include a data analysis system. The data analysis system may receive integrated data repository requests from one or more computing devices, such as an example computing device. The one or more integrated data repository requests may cause data to be retrieved from the integrated data repository. In various examples, the one or more integrated data repository requests may cause data to be retrieved from one or more datasets generated by the data pipeline system. The integrated data repository requests may specify the data to be retrieved from the integrated data repository and/or the one or more datasets generated by the data pipeline system. In one or more additional examples, the integrated data repository requests may include one or more prebuilt queries that correspond to computer-executable instructions that retrieve a specified set of data from the integrated data repository and/or one or more datasets generated by the data pipeline system.

In response to one or more integrated data repository requests, the data analysis system may analyze data retrieved from at least one of the integrated data repository or one or more datasets generated by the data pipeline system to generate data analysis results. The data analysis results may be sent to one or more computing devices, such as example computing devices. Although the illustrative example of hows that the one or more integrated data repository requests from one computing device and the data analysis results being sent to another computing device, in one or more additional implementations, the data analysis results may be received by a same computing device that sent the one or more integrated data repository requests. The data analysis results may be displayed by one or more user interfaces rendered by the computing device or the computing device.

In one or more examples, the data analysis system may implement at least one of one or more machine learning techniques or one or more statistical techniques to analyze data retrieved in response to one or more integrated data repository requests. In one or more examples, the data analysis system may implement one or more artificial neural networks to analyze data retrieved in response to one or more integrated data repository requests. To illustrate, the data analysis system may implement at least one of one or more convolutional neural networks or one or more residual neural networks to analyze data retrieved from the integrated data repository in response to one or more integrated data repository requests. In at least some examples, the data analysis system may implement one or more random forests techniques, one or more support vector machines, or one or more Hidden Markov models to analyze data retrieved in response to one or more integrated data repository requests. One or more statistical models may also be implemented to analyze data retrieved in response to one or more integrated data repository requests to identify at least one of correlations or measures of significance between characteristics of individuals. For example, log rank tests may be applied to data retrieved in response to one or more integrated data repository requests. In addition, Cox proportional hazards models may be implemented with respect to date retrieved in response to one or more integrated data repository requests. Further, Wilcoxon signed rank tests may be applied to data retrieved in response to one or more integrated data repository requests. In still other examples, a z-score analysis may be performed with respect to data retrieved in response to one or more integrated data repository requests. In still additional examples, a Kaplan Meier analysis may be performed with respect to data retrieved in response to one or more integrated data repository requests. In at least some examples, one or more machine learning techniques may be implemented in combination with one or more statistical techniques to analyze data retrieved in response to one or more integrated data repository requests.

In one or more illustrative examples, the data analysis system may determine a rate of survival of individuals in which lung cancer is present in response to one or more treatments. In one or more additional illustrative examples, the data analysis system may determine a rate of survival of individuals having one or more genomic region mutations in which lung cancer is present in response to one or more treatments. In various examples, the data analysis system may generate the data analysis results in situations where the data retrieved from at least one of the integrated data repository or the one or more datasets generated by the data pipeline system satisfies one or more criteria. For example, the data analysis system may determine whether at least a portion of the data retrieved in response to one or more integrated data repository requests satisfies a threshold confidence level. In situations where the confidence level for at least a portion of the date retrieved in response to one or more integrated data repository requests is less than a threshold confidence level, the data analysis system may refrain from generating at least a portion of data analysis results. In scenarios where the confidence level for at least a portion of the data retrieved in response to one or more integrated data repository requests is at least a threshold confidence level, the data analysis system may generate at least a portion of the data analysis results. In various examples, the threshold confidence level may be related to the type of data analysis results being generated by the data analysis system.

In one or more illustrative examples, the data analysis system may receive an integrated data repository request to generate data analysis results that indicate a rate of survival of one or more individuals. In these instances, the data analysis system may determine whether the data stored by the integrated data repository and/or by one or more datasets generated by the data pipeline system satisfies a threshold confidence level, such as a Gold standard confidence level. In one or more additional examples, the data analysis system may receive an integrated data repository request to generate data analysis results that indicate a treatment received by one or more individuals. In these implementations, the data analysis system may determine whether the data stored by the integrated data repository and/or by one or more datasets generated by the data pipeline system satisfies a lower threshold confidence level, such as a Bronze standard confidence level.

In one or more additional illustrative examples, the data analysis system may receive an integrated data repository request to determine individuals having one or more genomic mutations and that have received one or more treatments for a biological condition. Continuing with this example, the data analysis system can determine a survival rate of individuals with the one or more genomic mutations in relation to the one or more treatments received by the individuals. The data analysis system can then identify based on the survival rate of individuals and effectiveness of treatments for the individuals in relation to genomic mutations that may be present in the individuals. In this way, health outcomes of individuals may be improved by identifying prospective treatments that may be more effective for populations of individuals having one or more genomic mutations than current treatments being provided to the individuals.

Described herein is a framework corresponding to an arrangement of data tables in an integrated data repository, according to one or more implementations. In the illustrative example of the framework includes a data repository schema that includes a first data table, a second data table, a third data table, a fourth data table a fifth data table, a sixth data table, and a seventh data table. Although the illustrative example of seven data tables, in additional implementations, the data repository schema may include more data tables or fewer data tables. The data repository schema may also include links between the data tables. The links between the data tables may indicate that information retrieved from one of the data tables results in additional information stored by one or more additional data tables to be retrieved. Additionally, not all the data tables may be linked to each of the other data tables. In the illustrative example of the first data table is logically coupled to the second data table by a first link and the third data table is logically coupled to the second data table by a second link. The second data table is also logically coupled to the fourth data table by a third link, the second data table is logically coupled to the fifth data table by a fourth link, and the second data table is logically coupled to the sixth data table by a fifth link. In addition, the fifth data table is logically coupled to the sixth data table by a sixth link and the sixth data table is logically coupled to the seventh data table by a seventh link. Further, the seventh data table is logically coupled to the fourth data table by an eighth link. In various examples, as data tables are added to and/or removed from the data repository schema, additional links between data tables may be added to or removed from the data repository schema. In one or more illustrative examples, the integrated data repository may store data tables according to the data repository schema for at least a portion of the individuals for which the data integration system obtained information from a combination of at least two of the electronic medical record, including for example, patient information, health insurance claims, lifestyle data, repository, the molecular data repository, and the one or more additional data repositories. As a result, the integrated data repository may store respective instances of the data tables according to the data repository schema for thousands, tens of thousands, up to hundreds of thousands or more individuals.

In one or more examples, the first data table may store data corresponding to genomics and genomics testing for individuals. For example, the first data table may include columns that include information corresponding to a panel used to generate genomics data, mutations of genomic regions, types of mutations, copy numbers of genomic regions, coverage data indicating numbers of nucleic acid molecules identified in a sample having one or more mutations, testing dates, and patient information. The first data table may also include one or more columns that include health insurance data codes that may correspond to one or more diagnosis codes. Additionally, the information in the first data table may include at least one identifier for an individual that is associated with an instance of the first data table.

The second data table may store data related to one or more patient visits by individuals to one or more healthcare providers. The third data table may store information corresponding to respective services provided to individuals with respect to one or more patient visits to one or more healthcare providers indicated by the second data table. To illustrate, an individual may visit a healthcare provider and multiple services may be performed with respect to the individual at the visit. A second data table may include columns indicating information for each of the multiple services performed during the patient visit. Multiple third data tables may be generated with respect to the patient visit that include columns indicating information on a more granular level for a respective service provided during the patient visit than the information stored by the second data table related to the patient visit. For example, the second data table may include multiple columns indicating a health insurance code for different services provided to an individual during a patient visit and a third data table related to one of the services may include multiple columns for additional health insurance codes that correspond to additional information related to the respective services. The second data table and the third data table(s) for a patient visit may indicate one or more dates of service corresponding to the patient visit.

The fourth data table may include columns that indicate information about individuals for which information is stored by the integrated data repository. For example, the fourth data table may include columns that indicate information related to at least one of a location of an individual, a gender of an individual, a date of birth of an individual, a date of death of an individual (if applicable), or one or more keys associated with the individual. In one or more examples, the fourth data table may include one or more columns related to whether erroneous data has been identified for an individual. In various examples, a single fourth data table may be generated for respective individuals. Thus, the data repository schema may include multiple instances of the fourth data table, such as thousands, tens of thousands, up to hundreds of thousands or more.

The fifth data table may include columns that indicate information related to a health insurance company or governmental entity that made payment for one or more services provided to respective individuals. For example, the fifth data table may include one or more payer identifiers. The sixth data table may include columns that include information corresponding to health insurance coverage information for respective individuals. In one or more examples, the sixth data table may include columns indicating the presence of medical coverage for an individual, the presence of pharmacy coverage for an individual, and a type of health insurance plan related to the individual, such as health maintenance organization (HMO), preferred provider organization (PPO), and the like.

The seventh data table may include columns that indicate information related to pharmaceutical treatments obtained by a respective individual. In one or more examples, the seventh data table may include one or more columns indicating health insurance codes corresponding to pharmaceutical treatments that are available via a pharmacy. The health insurance codes may correspond to individual pharmaceutical treatments. Additionally, the health insurance codes may indicate a diagnosis of a biological condition with respect to an individual. The seventh data table may also include additional information, such as at least one of dosage amounts, number of days' supply, quantity dispensed, number of refills authorized, dates of service, or information related to the individual receiving the pharmaceutical treatment.

In various examples, the data repository schema may provide results of analysis of the information stored by the data tables in a more efficient manner than typical data repository schemas. For example, the logical connections between the data tables are arranged to efficiently retrieve data that is related across the different data tables. In situations where the data tables are arranged in a serial manner and/or in situations where a greater number of the data tables are logically connected, retrieving data from the integrated data repository from one or more of the data tables to responds to a request for information from the integrated data repository will be less efficient than in situations where the data repository schema is implemented.

Described herein is an architecture to generate one or more datasets from information retrieved from a data repository that integrates health related data from a number of sources, according to one or more implementations. The architecture may include the data integration and analysis system and the integrated data repository. Additionally, the data integration and analysis system may include at least the data pipeline system and the data analysis system. The data pipeline system may include a number of sets of data processing instructions that are executable to generate respective datasets that may be analyzed by the data analysis system in response to an integrated data repository request to generate data analysis results.

The data pipeline system may include first data processing instructions, second data processing instructions, up to Nth data processing instructions. The data processing instructions, may be executable by one or more processing units to perform a number of operations to generate respective datasets using information obtained from the integrated data repository. In one or more illustrative examples, the data processing instructions, may include at least one of software code, scripts, API calls, macros, and so forth. The first data processing instructions may be executable to generate a first dataset. In addition, the second data processing instructions may be executable to generate a second dataset. Further, the Nth data processing instructions may be executable to generate an Nth dataset. In various examples, after the data integration and analysis system generates the integrated data repository, the data pipeline system may cause the data processing instructions, to be executed to generate the datasets. In one or more examples, the datasets, may be stored by the integrated data repository or by an additional data repository that is accessible to the data integration and analysis system. At least a portion of the data processing instructions may analyze health insurance codes to generate at least a portion of the datasets. Additionally, at least a portion of the data processing instructions may analyze genomics data to generate at least a portion of the datasets.

In one or more examples, the first data processing instructions may be executable to retrieve data from one or more first data tables stored by the integrated data repository. The first data processing instructions may also be executable to retrieve data from one or more specified columns of the one or more first data tables. In various examples, the first data processing instructions may be executable to identify individuals that have a health insurance code stored in one or more column and row combinations that correspond to one or more diagnosis codes. The first data processing instructions may then be executable to analyze the one or more diagnosis codes to determine a biological condition for which the individuals have been diagnosed. In one or more illustrative examples, the first data processing instructions may be executable to analyze the one or more diagnosis codes with respect to a library of diagnosis codes that indicates one or more biological conditions that correspond to respective diagnosis codes. The library of diagnosis codes may include hundreds up to thousands of diagnosis codes. The first data processing instructions may also be executable to determine individuals diagnosed with a biological condition by analyzing timing information of the individuals, such as dates of treatment, dates of diagnosis, dates of death, one or more combinations thereof, and the like.

The second data processing instructions may be executable to retrieve data from one or more second data tables stored by the integrated data repository. The second data processing instructions may also be executable to retrieve data from one or more specified columns of the one or more second data tables. In various examples, the second data processing instructions may be executable to identify individuals that have a health insurance code stored in one or more columns and row combinations that correspond to one or more treatment codes. The one or more treatment codes may correspond to treatments obtained from a pharmacy. In one or more additional examples, the one or more treatment codes may correspond to treatments received by a medical procedure, such as an injection or intravenously. The second data processing instructions may be executable to determine one or more treatments that correspond to the respective health insurance codes included in the one or more second data tables by analyzing the health insurance code in relation to a predetermined set of information. The predetermined set of information may include a data library that indicates one or more treatments that correspond to one out of hundreds up to thousands of health insurance codes. The second data processing instructions may generate the second dataset to indicate respective treatments received by a group of individuals. In one or more illustrative examples, the group of individuals may correspond to the individuals included in the first dataset. The second dataset may be arranged in rows and columns with one or more rows corresponding to a single individual and one or more columns indicating the treatments received by the respective individual.

The Nth processing instructions (where N may be any positive integer) may be executable to generate the Nth dataset by combining information from a number of previously generated datasets, such as the first dataset and the second dataset. In addition, the Nth processing instructions may be executable to generate the Nth dataset to retrieve additional information from one or more additional columns of the integrated data repository and incorporate the additional information from the integrated data repository with information obtained from the first dataset and the second dataset. For example, the Nth processing instructions may be executable to identify individuals included in the first dataset that are diagnosed with a biological condition and analyze specified columns of one or more additional data tables of the integrated data repository to determine dates of the treatments indicated in the second dataset that correspond to the individuals included in the first dataset. In one or more further examples, the Nth processing instructions may be executable to analyze columns of one or more additional data tables of the integrated data repository to determine dosages of treatments indicated in the second dataset received by the individuals included in the first dataset. In this way, the Nth processing instructions may be executable to generate an episodes of care dataset based on information included in a cohort dataset and a treatments dataset.

In one or more illustrative examples, in response to receiving an integrated data repository request, the data analysis system may determine one or more datasets that correspond to the features of the query related to the integrated data repository request. For example, the data analysis system may determine that information included in the first dataset and the second dataset is applicable to responding to the integrated data repository request. In these scenarios, the data analysis system may analyze at least a portion of the data included in the first dataset and the second dataset to generate the data analysis results. In one or more additional examples, the data analysis system may determine different datasets to respond to different queries included in the integrated data repository request in order to generate the data analysis results.

The use of specific sets of data processing instructions to generate respective data sets may reduce the number of inputs from users of the data integration and analysis system as well as reduce the computational load, such as the amount of processing resources and memory, utilized to process integrated data repository requests. For example, without the specific architecture of the data pipeline system, each time an integrated data repository request is received, the data utilized to respond to the integrated data repository request is assembled from the data repository. In contrast, by implementing the data pipeline system to execute the data processing instruction to generate the datasets the data needed to respond to various integrated data repository requests has already been assembled and may be accessed by the data analysis system to respond to the integrated data repository request. Thus, the computing resources used to respond to the integrated data repository request by implementing the data pipeline system to generate the datasets are less than typical systems that perform an information parsing and collecting process for each integrated data repository request. Further, in situations where the data pipeline system has not been implemented, users of the data integration and analysis system may need to submit multiple integrated data repository request in order to analyze the information that the users are intending to have analyzed either because the ad hoc collection of data to respond to an integrated data repository request in typical systems is inaccurate or because the data analysis system is called upon multiple times to perform an analysis of information in typical systems that may be performed using a single integrated data repository request when the data pipeline system is implemented.

Described herein is an architecture to generate an integrated data repository that includes de-identified electronic medical record, including for example, patient information, health insurance claims, lifestyle data, and de-identified genomics data it, according to one or more implementations. The architecture may include the data integration and analysis system, the electronic medical record, including for example, patient information, health insurance claims, lifestyle data, repository, and the molecular data repository. The data integration and analysis system may obtain patient information from the molecular data repository. The patient information may include genomics data for individuals having data stored by the molecular data repository. The genomics data may indicate results of one or more nucleic acid sequencing operations that analyze sequences of nucleic acid molecules included in a sample obtained from the individuals with respect to one or more target genomic regions. In one or more examples, the sample may be obtained from tissue of one or more individuals. In one or more additional examples, the sample may be obtained from fluid of one or more individuals, such as blood or plasma. The one or more target genomic regions may correspond to genomic regions that correspond to the presence of one or more biological conditions. For example, the target regions may correspond to genomic regions of a reference genome having mutations that are present in individuals in which a biological condition is present. In one or more illustrative examples, the target regions may correspond to genomic regions of a reference human genome in which one or more mutations are present in individuals in which one or more forms of cancer are present. The patient information may also include information indicating personal information about individuals with data stored by the molecular data repository and information corresponding to the testing and analysis performed on samples provided by individuals.

The data integration and analysis system may perform a de-identification process that anonymizes personal information obtained from the molecular data repository. The data integration and analysis system may implement one or more computational techniques as part of the de-identification process to anonymize data related to individuals stored by the molecular data repository such that the de-identified data protects the privacy of the individuals and is in compliance with one or more privacy regulation frameworks. The de-identification process may include, at, accessing tokens. In various examples, the tokens may comprise an alphanumeric string of characters. In one or more examples, the tokens may be generated by the data integration and analysis system. In one or more additional examples the tokens may be generated by a third-party and obtained by the data integration and analysis system.

The tokens may be generated using one or more hash functions in relation to a subset of the patient information. To illustrate, for individuals that have information stored by the molecular data repository, the tokens may be generated using a combination of at least a portion of a first name of the respective individuals, at least a portion of the last name of the respective individuals, at least a portion of a date of birth of the respective individuals, a gender of the individuals, and at least a portion of a location identifier of the respective individuals. The de-identification process may also include, at, generating identifiers for individuals that have data stored by the molecular data repository. The identifiers may be generated by the data integration and analysis system using one or more hash functions that are different from the one or more hash functions used to generate the tokens. In one or more illustrative examples, the data integration and analysis system may generate an intermediate version of respective identifiers using one or more hash function and then apply one or more salting techniques to the intermediate versions of the identifiers to generate final versions of the identifiers. A salt function includes a function configured to add at least one random bit to each intermediate identifier to generate a respective final identifier. In various examples, the data integration and analysis system may generate the identifiers at using at least a portion of the information for respective individuals stored by the molecular data repository. In one or more illustrative examples, the identifiers may be generated based on a patient identifier included in the patient information. The identifiers generated by the data integration and analysis system may be unique for respective individuals having data stored by the molecular data repository.

At operation, the data integration and analysis system may generate modified patient information based on the identifiers. The modified patient information may include genomics data related to individuals associated with the molecular data repository and the identifiers of the respective individuals. The modified patient information may have a data structure. The data structure may include a column that includes respective identifiers of individuals associated with the molecular data repository and a number of columns that include genomics data related to the individuals, such as identifiers of one or more genes, alterations to the one or more genes, type of alteration to the genes, and so forth.

The data integration and analysis system may generate a token file. The token file may include first tokens accessed at operation for respective individuals having data stored by the molecular data repository. The token file may have a data structure that includes a number of columns that include information for respective individuals. The data structure may include a column indicating respective identifiers generated by the data integration and analysis system and columns indicating one or more first tokens associated with the respective identifiers. The data integration and analysis system may send the token file to a electronic medical record, including for example, patient information, health insurance claims, lifestyle data, management system that is coupled to the electronic medical record, including for example, patient information, health insurance claims, lifestyle data, repository. The electronic medical record, including for example, patient information, health insurance claims, lifestyle data, management system may analyze the first tokens with respect to corresponding second tokens. The second tokens may be accessed by or generated by the electronic medical record, including for example, patient information, health insurance claims, lifestyle data, management system. The second tokens may be generated using a same or similar subset of information for individuals having data stored in the electronic medical record, including for example, patient information, health insurance claims, lifestyle data, repository as the subset of the patient information. For example, the second tokens may be generated using a combination of at least a portion of a first name of the respective individuals, at least a portion of the last name of the respective individuals, at least a portion of a date of birth of the respective individuals, a gender of the individuals, and at least a portion of a location identifier of the respective individuals.

By integrating the genomics data records for the group of individuals with the health insurance claims records, the data integration and analysis system may determine correlations between the presence of one or more biomarkers that are present in the genomics data records with other characteristics of individuals that are indicated by the electronic medical record, including for example, patient information, health insurance claims, lifestyle data, records that existing systems are typically unable to determine. For example, the data integration and analysis system may determine one or more genomic characteristics of individuals that correspond to treatments received by individuals, timing of treatments, dosages of treatments, diagnoses of individuals, smoking status, presence of one or more biological conditions, presence of one or more symptoms of a biological condition, one or more combinations thereof, and the like. Based on the correlations determined by the data integration and analysis system using the integrated data repository, cohorts of individuals that may benefit from one or more treatments may be identified that would not have been identified in existing systems. In one or more examples, the processes and techniques implemented to integrate the health insurance claims records and the genomics claims records in order to generate the integrated data repository may be complex and implement efficiency-enhancing techniques, systems, and processes in order to minimize the amount of computing resources used to generate the integrated data repository.

In one or more illustrative examples, the data pipeline system may access information stored by the integrated data repository to generate datasets that include a number of additional data records that include information related to at least a portion of the group of individuals. In an illustrative example, the additional data record includes information indicating whether individuals are included in a cohort of individuals in which lung cancer is present. The data pipeline system may execute a plurality of different sets of data processing instructions to determine a cohort of the group of individuals in which lung cancer is present. In various examples, the additional data record may indicate information used to determine a status of an individual with respect to lung cancer, such as one or more transaction insurance identifier, one or more international classification of diseases (ICD) codes, and one or more health insurance transaction dates. In addition to including a column that indicates whether an individual is included in the lung cancer cohort, the additional data record may include a column indicating a confidence level of the status of the individual with respect to the presence of lung cancer.

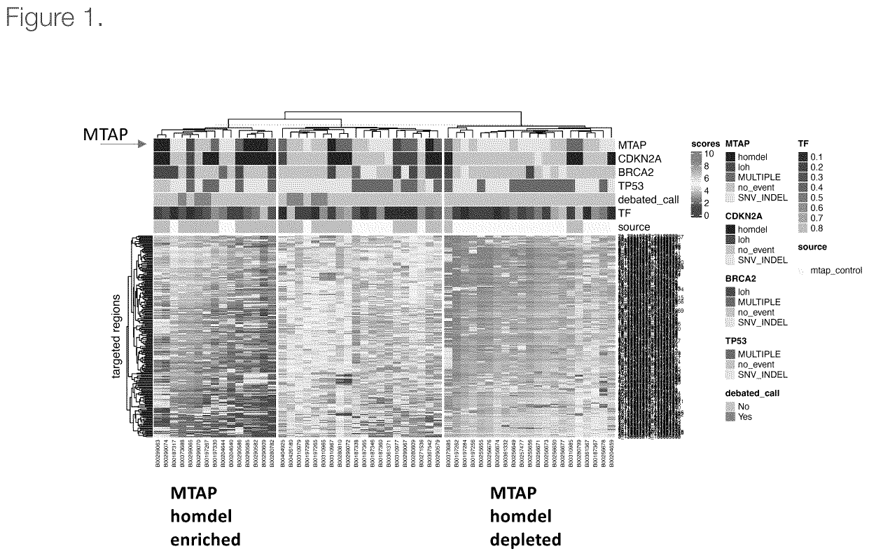

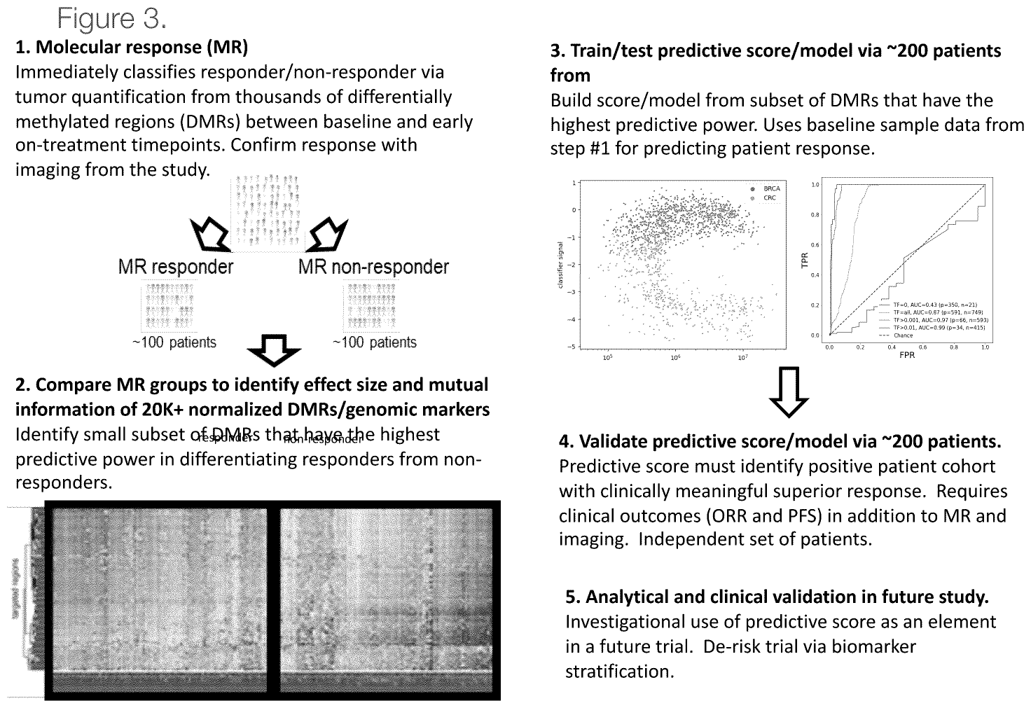

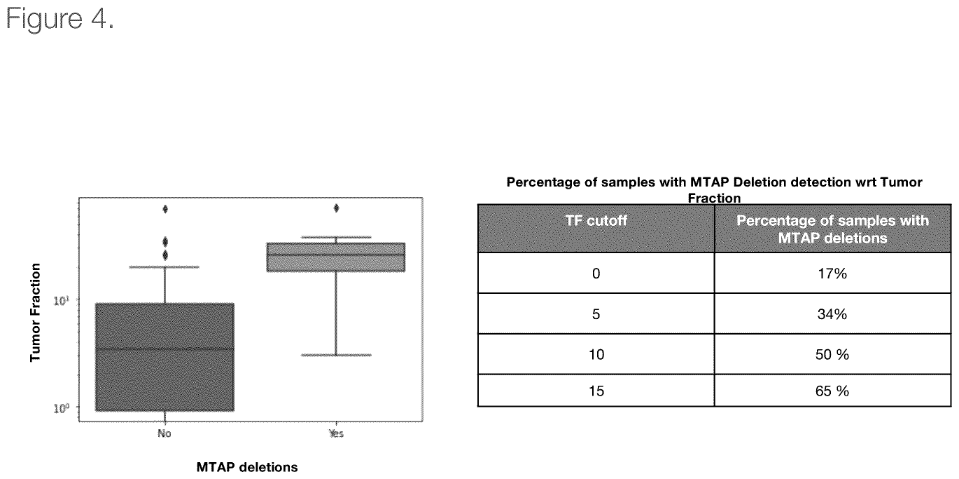

Described herein is a schematic diagram of a computing architecture 600 to incorporate medical records data into an integrated data repository. In various examples, at least a portion of the operations of the computing architecture may be performed by the data integration and analysis system of FIGS. 1, 3, and 4. In one or more examples, at least a portion of the operations of the computing architecture may be performed by one or more additional computing systems that are at least one of controlled, maintained, or implemented by a service provider that also at least one of controls, maintains, or implements the data integration and analysis system. In one or more additional examples, at least a portion of the operations of the computing architecture may be performed by a number of servers in a distributed computing environment.

The computing architecture may include a medical records data repository. The medical records data repository may store medical records data from a number of individuals. The medical records data may include imaging information, laboratory test results, diagnostic test information, clinical observations, dental health information, notes of healthcare practitioners, medical history forms, diagnostic request forms, medical procedure order forms, medical information charts, one or more combinations thereof, and so forth. In various examples, for a given individual, the medical records data repository may store information obtained from one or more healthcare practitioners that is related to the individual.

The computing architecture may perform operation that includes obtaining data packages from the medical records data repository. In one or more examples, the data packages may be obtained in response to one or more requests sent to the medical records data repository for medical records that correspond to one or more individuals. In one or more additional examples, the data packages may be obtained by the computing architecture using one or more application programming interface (API) calls. In one or more illustrative examples, a first data package, a second data package, up to an Nth data package may be obtained using the computing architecture. The individual data packages, may correspond medical records of a respective individual. For example, the first data package may include medical records of a first individual, the second data package may include medical records of a second individual, and the Nth data package may include medical records of a third individual.

Individual data packages, may include a number of components. In one or more examples, individual data packages, may include individual components that correspond to medical records from different healthcare providers. In one or more additional examples, the individual data packages, may include individual components that correspond to different parts of medical records that correspond to one or more healthcare providers. In an illustrative example the second data package may include a first component, a second component, up to an Nth component. In one or more illustrative examples, the first component may include a first portion of medical records of an individual, the second component may include a second portion of medical records of an individual, and the Nth component may include a third portion of medical records of an individual. In various examples, the first component may correspond to medical records of a first healthcare provider for the individual, the second component may correspond to medical records of a second healthcare provider for the individual, and the third component may correspond to medical records of a third healthcare provider for the individual. In one or more additional illustrative examples, the first component may include a first section of medical records of the individual, such as one or more forms related to a diagnostic test or procedure, and the second component may include a second section of medical records of the individual, such as a pathology report of the individual.

At operation, the computing architecture may preprocess individual data packages to identify a corpus of information to be analyzed. In one or more examples, the preprocessing of data packages obtained from the medical records data repository, may include transforming the data included in the data packages. For example, preprocessing the data packages may include transforming at least a portion of the data obtained from the medical records data repository to machine encoded information. To illustrate, preprocessing the data packages may include performing one or more optical character recognition (OCR) operations with respect to at least a portion of the data packages obtained from the medical records data repository. By converting at least a portion of the data packages obtained from the medical records data repository to machine encoded information, the data packages may be subjected to a number of operations, such as one or more parsing operations to identify one or more characters or strings of characters or one or more editing operations that are unable to be performed with respect to at least a portion of the data packages obtained from the medical records data repository.

In one or more examples, the preprocessing of individual data packages may include determining information included in individual data packages that is to be excluded from further analysis by the computing architecture. In various examples, one or more components of individual data packages may be excluded from a corpus of information to be analyzed. For example, with respect to the second data package, the computing architecture may determine that the first component is to be excluded from further analysis by the computing architecture. In one or more examples, the computing architecture may analyze the components, and/or with respect to one or more keywords to identify at least one of the components, and/or to exclude from further analysis by the computing architecture. In one or more illustrative examples, the computing architecture may parse the components, and/or to identify one or more keywords and in response to identifying the one or more keywords in a component, and/or, the computing architecture may determine to exclude the respective component, and/or from further analysis by the computing architecture. For example, the computing architecture may determine that the first component of the second data package is a test requisition form for one or more diagnostic procedures or tests. In these scenarios, the computing architecture may determine that the first component is to be excluded from further analysis by the computing architecture. Additionally, the computing architecture may determine that at least one of the second component and/or correspond to one or more pathology reports for an individual based on one or more keywords included in at least one of the second component or the Nth component. In these instances, the computing architecture may determine that at least a portion of the second component and/or at least a portion of the Nth component is to be included in the corpus of information to be further analyzed by the computing architecture.

In addition, a subset of the components of individual data packages obtained from the medical records data repository may be included in the corpus of information. In various examples, one or more additional operations may be performed to narrow the corpus of information. For example, one or more queries may be applied to a subset of information obtained from the medical records data repository. The one or more queries may extract information from the one or more data packages that satisfy the one or more queries. In at least some examples, the one or more queries may be a group of queries that are applied to individual components of a data package. In one or more illustrative examples, the group of queries may determine information to be included in the corpus of information and additional information that is to be excluded from the corpus of information. In one or more additional examples, one or more sections of at least one component of a data package may be excluded from the corpus of information.

In one or more additional illustrative examples, after determining that the first component is to be excluded from further analysis by the computing architecture, the computing architecture may then cause one or more queries to be implemented with respect to at least one the second component or the Nth component. In these scenarios, the one or more queries may determine that a section of the second component, such as a section that indicates family history for one or more biological conditions, is to be excluded from the corpus of information. In various examples, the one or more queries may be directed to identifying a number of keywords and/or combinations of keywords included in at least one of the second component or the Nth component. In these instances, the computing architecture may exclude from the corpus of information one or more portions of the individual components of the data packages that include one or more keywords or combinations of keywords. In one or more additional examples, the computing architecture may exclude from the corpus of information a number of words, a number of characters, and/or a number of symbols following one or more keywords that are included in one or more portions of the individual components of the data packages.

Further, at operation, the computing architecture may analyze the corpus of information to determine characteristics of individuals. In one or more examples, the computing architecture may analyze the corpus of information to determine individuals that have one or more phenotypes. In various examples, the computing architecture may analyze the corpus of information to determine one or more biomarkers that are indicative of a biological condition. For example, the computing architecture may analyze the corpus of information to determine individuals having one or more genetic characteristics. The one or more genetic characteristics may include at least one of one or more variants of a genomic region that correspond to a biological condition. In one or more illustrative examples, the one or more genetic characteristics may correspond to one or more variants of a genomic region that correspond to a type of cancer. In one or more additional illustrative examples, the one or more biomarkers may correspond to levels of an analyte being outside of a specified range. To illustrate, the computing architecture may analyze the corpus of information to determine individuals having levels of one or more proteins and/or levels of one or more small molecules present that are indicative of a biological condition. In these scenarios, the computing architecture may analyze results of laboratory tests to determine levels of analytes of individuals. In one or more additional examples, the computing architecture may analyze the corpus of information to determine individuals in which one or more symptoms are present that are indicative of a biological condition. In one or more further examples, the computing architecture may analyze imaging information included in the corpus of information to determine individuals in which one or more biomarkers are present.

In one or more examples, the computing architecture may implement one or more machine learning techniques to analyze the corpus of information. For example, the computing architecture may implement one or more artificial neural networks, such as at least one of one or more convolutional neural networks or one or more residual neural networks to analyze the corpus of information. The computing architecture may also implement at least one of one or more random forests techniques, one or more hidden Markov models, or one or more support vector machines to analyze the corpus of information.

In at least some implementations, the computing architecture may analyze the corpus of information by performing one or more queries with respect to the corpus of information. The one or more queries may correspond to one or more keywords and/or combinations of keywords. The one or more keywords and/or combinations of keywords may correspond to at least one of characters or symbols that correspond to one or more biological conditions. To illustrate, a keyword may correspond to characters related to a mutation of a genomic region, such as HER2. In one or more additional illustrative examples, one or more criteria may be associated with combinations of keyworks. To illustrate, a criterion that corresponds to a combination of keywords may include a number of words being present within a specified distance of one another in a portion of the corpus of information for an individual, such as the words fatigue, blood pressure, and swelling occurring within characters of one another. In these instances, the computing architecture may parse the corpus of information for the one or more keywords and/or combinations of keywords. In various examples, in response to determining that the one or more keywords and/or combinations of keywords are present in accordance with one or more criteria, the computing architecture may determine that a biological condition is present with respect to a given individual.

In one or more additional examples, the one or more queries may be image-based and the computing architecture may analyze images included in the corpus of information with respect to template images. The template images may be generated based on analyzing a number of images in which a biological condition is present and aggregating the number of images into a template image. In these scenarios, the computing architecture may analyze images included in the corpus of information with respect to one or more template images to determine a measure of similarity between the images included in the corpus of information and the template images. In situations where the measure of similarity for an individual is at least a threshold value, the computing architecture may determine that a characteristic of a biological condition is present in the individual.

After determining individuals having one or more characteristics, the computing architecture may, at operation, generate data structures that store data for individuals having the one or more characteristics. In one or more examples, the computing architecture may generate data tables that indicate individuals having an individual characteristics and/or individuals having a group of characteristics. For example, the computing architecture may generate a first data table and a second data table. The first data table may indicate individuals having one or more first characteristics and the second data table may indicate individuals having one or more second characteristics. In one or more illustrative examples, the first data table may indicate individuals having one or more first biomarkers for a biological condition and the second data table may indicate individual having one or more second biomarkers for the biological condition. The one or more first biomarkers may correspond to one or more first genomic variants that are associated with the biological condition and the one or more second biomarkers may correspond to one or more second genomic variants that are associated with the biological condition. In various examples, the data tables, may indicate whether or not the one or more characteristics associated with the individual data tables, are present with respect to individual individuals. To illustrate, the first data table may include a first indication for individuals in which one or more first genomic variants are present and a second indication for individuals in which the one or more first genomic variants are not present. In one or more additional examples, the first data table may indicate smoking status of individuals and the second data table may indicate whether or not individual individuals have received one or more treatments for a biological condition.

In one or more illustrative examples, the first data table and the second data table may have rows that correspond to individual individuals. In at least some examples, an individual identifier may be present in individual rows. The individual identifier may include at least one of alphanumeric characters or symbols that correspond to an individual. In various examples, the individual identifier may be present in a data package that corresponds to an individual. Columns of the first data table and the second data table may indicate a status of individual individuals with respect to one or more characteristics. For example, the columns of the data tables, may include an identifier that includes at least one of alphanumeric characters or symbols that indicate the presence or absence of one or more characteristics for a given individual. Further, although the illustrative example of includes a first data table and a second data table, the computing architecture may generate more data tables or fewer data tables.

At operation, the computing architecture may store the data structures in an additional data repository. For example, the computing architecture may store at least the first data tale and/or the second data table in an intermediate data repository. In various examples, the first data table and the second data table may be temporarily stored in the intermediate data repository. In one or more illustrative examples, the first data table and the second data table may be stored in the intermediate data repository before being added to the integrated data repository. In one or more examples, the integrated data repository may be periodically generated and/or updated. In these scenarios, data structures generated by the computing architecture based on analyzing the corpus of information may be stored in the intermediate data repository until a time when the integrated data repository is to be at least one of generated or updated.

Prior to adding data structures stored by the intermediate data repository to the integrated data repository, the computing architecture may perform one or more de-identification processes at operation. The data structures stored by the intermediate data repository may be de-identified in order to preserve the privacy of individuals. The one or more de-identification processes may include applying one or more electronically implemented cryptographic techniques to information of individuals included in the data structures stored by the intermediate data repository. In one or more examples, the computing architecture may generate tokens that correspond to individual individuals that have information stored in data structures of the intermediate data repository. The tokens may be generated by applying one or more hash functions to information related to individual individuals. In one or more examples, the one or more de-identification processes may include applying a salt function to information corresponding to individual individuals to generate tokens for the individual individuals. In various examples, the one or more cryptographic techniques applied to de-identify the data structures stored by the intermediate data repository may be the same or similar to those applied to information obtained from the electronic medical record, including for example, patient information, health insurance claims, lifestyle data, repository.

At operation, the computing architecture may store the de-identified data structures in conjunction with the integrated data repository. For example, the information stored in the intermediate data repository for a given individual may be stored in conjunction with additional information about the given individual in the integrated data repository. To illustrate, the integrated data repository may store information for a given individual obtained from at least two of the molecular data repository, obtained from the electronic medical record, including for example, patient information, health insurance claims, lifestyle data, repository, and obtained from the intermediate data repository. In this way, information about a given individual obtained from a number of disparate data repositories may be stored in the integrated data repository. As a result, information about individuals that is obtained from the different data repositories may be analyzed together rather than analyzed separately as with many existing systems.

In various examples, the information stored by the intermediate data repository may be used to validate one or more determinations made by the data integration and analysis system. For example, the data integration and analysis system may analyze information obtained from the electronic medical record, including for example, patient information, health insurance claims, lifestyle data, repository and the molecular data repository to determine characteristics of individuals. The data integration and analysis system may then analyze information obtained from the intermediate data repository to determine whether the predicted characteristics identified from the information obtained from the electronic medical record, including for example, patient information, health insurance claims, lifestyle data, repository and from the molecular data repository correspond to the characteristics for the same individuals with respect to information stored by the intermediate data repository.

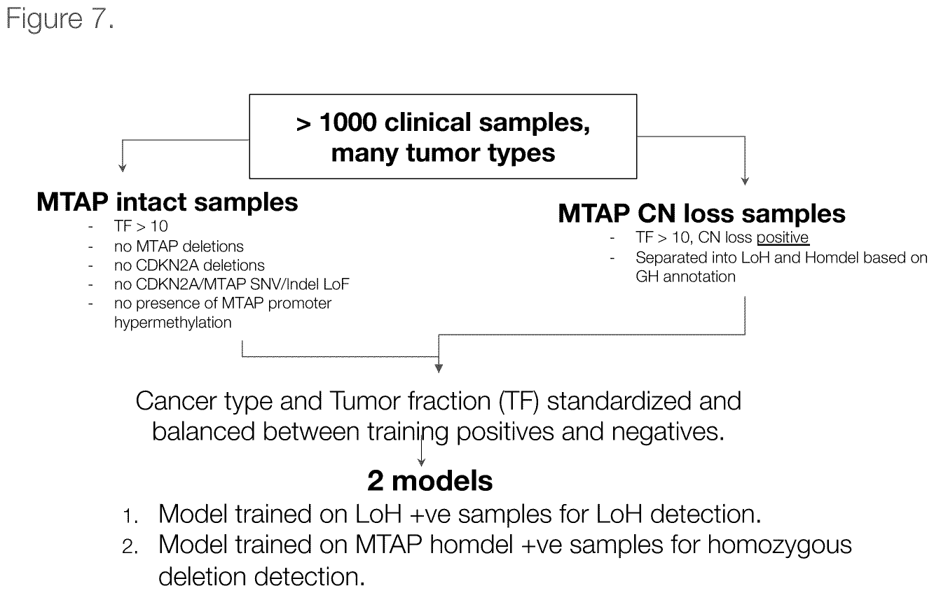

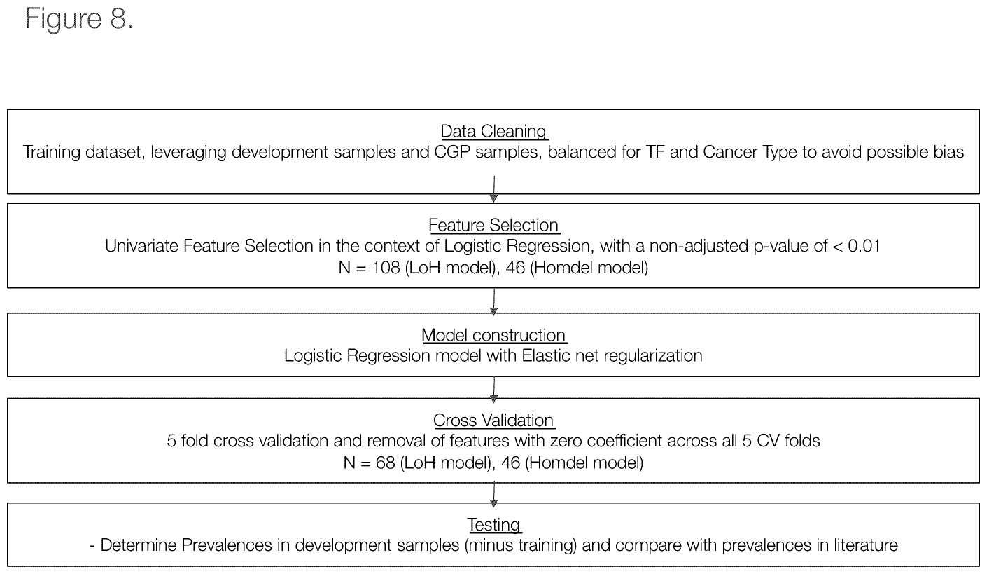

The one or more cryptographic techniques applied to de-identify the data structures stored by the intermediate data repository may utilize the same or similar information that was used to generate at least one of the first tokens or the second tokens. For example, the operation may implement one or more cryptographic techniques using a combination of at least a portion of a first name of the respective individuals, at least a portion of the last name of the respective individuals, at least a portion of a date of birth of the respective individuals, a gender of the individuals, and at least a portion of a location identifier of the respective individuals to de-identify the data structures of the intermediate data repository. By utilizing the same or similar cryptographic techniques and the same or similar subset of information to de-identify the data structures stored by the intermediate data repository as were used to generate at least one of the first tokens or the second tokens, the information stored by the intermediate data repository may be synchronized with information for the same individuals that have information stored in the integrated data repository. Both the integrated data repository and the intermediate data repository may store information for thousands, tens of thousands, up to millions of individuals. Thus, without the ability to synchronize the individuals having records stored by the integrated data repository and the intermediate data repository through the use of a specified cryptographic protocol as described herein, the data structures of the integrated data repository and the data structures of the intermediate data repository that are associated with a same individual may not be stored in a manner such that the information stored by the integrated data repository and the information stored by the intermediate data repository may be retrieved together for a given individual, which may lead to inaccurate information being provided by the data integration and analysis system. The absence of a specified cryptographic protocol as described herein may also lead to the use of more computing resources to determine the information stored in the integrated data repository from other data sources and the information stored by the intermediate data repository that correspond to a given individual. FIGS. 7 and 8 illustrate example processes to generate an integrated data repository and generate datasets used in the analysis of information stored by the integrated data repository. The example processes are illustrated as collections of blocks in logical flow graphs, which represent sequences of operations that may be implemented in hardware, software, or a combination thereof. The blocks are referenced by numbers. In the context of software, the blocks represent computer-executable instructions stored on one or more computer-readable media that, when executed by one or more processing units (such as hardware microprocessors), perform the recited operations. Generally, computer-executable instructions include routines, programs, objects, components, data structures, and the like that perform particular functions or implement particular data types. The order in which the operations are described is not intended to be construed as a limitation, and any number of the described blocks may be combined in any order and/or in parallel to implement the process.

Described herein is a data flow diagram of an example process to generate an integrated data repository that stores electronic medical record, including for example, patient information, health insurance claims, lifestyle data, and genomics data, according to one or more implementations. At operation, the process may include generating a data file that includes tokens generated using a first hash function. Individual tokens may correspond to a respective individual of a group of individuals having data stored by a molecular data repository. In one or more examples, an individual having data stored by the molecular data repository may be associated with one or more tokens. The tokens may be generated by applying one or more first hash functions to a subset of information corresponding to the group of individuals stored by the genomics data repository. In various examples, individual tokens may be generated by applying one or more first hash functions to one or more combinations of at least a portion of a first name of a respective individual of the group of individuals, at least a portion of a second name of a respective individual of the group of individuals, a location identifier of a respective individual of the group of individuals, a gender of a respective individual of the group of individuals, and a date of birth of a respective individual of the group of individuals. In one or more illustrative examples, the tokens may be generated by a data integration and analysis system that is coupled to the genomics data repository. In one or more additional illustrative examples, the tokens may be generated by a third-party system and accessed by a data integration and analysis system coupled to the molecular data repository. The process may also include, at operation, sending the data file to a electronic medical record, including for example, patient information, health insurance claims, lifestyle data, management system. The electronic medical record, including for example, patient information, health insurance claims, lifestyle data, management system may match the tokens included in the data file with second tokens accessed by the health insurance data management system and generated based on information stored by a electronic medical record, including for example, patient information, health insurance claims, lifestyle data, repository.

In addition, at operation, the process may include obtaining, from the electronic medical record, including for example, patient information, health insurance claims, lifestyle data, management system, in response to the data file, first data corresponding to the group of individuals, where the first data includes electronic medical record, including for example, patient information, health insurance claims, lifestyle data. In some implementations, affirmative consent is obtained from the members of the group of individuals for their data to be transferred from the electronic medical record, including for example, patient information, health insurance claims, lifestyle data, management system. In one or more examples, the data is transferred in an anonymized format, such that the data may not be traced back to an individual member. The electronic medical record, including for example, patient information, health insurance claims, lifestyle data, management system may be coupled to a electronic medical record, including for example, patient information, health insurance claims, lifestyle data, repository that stores health insurance claims information for a number of individuals. In one or more examples, the electronic medical record, including for example, patient information, health insurance claims, lifestyle data, management system may analyze the tokens of the data file with respect to additional tokens generated by the electronic medical record, including for example, patient information, health insurance claims, lifestyle data, management system. The additional tokens may be generated based on a same set of information used to generate the tokens included in the data file. However, an individual's identity may not be determined based on a token. In various examples, the electronic medical record, including for example, patient information, health insurance claims, lifestyle data, management system may match tokens included in the data file with additional tokens generated based on information stored by the electronic medical record, including for example, patient information, health insurance claims, lifestyle data, repository to determine individuals having information stored by the electronic medical record, including for example, patient information, health insurance claims, lifestyle data, repository that also have information stored by the genomics data repository. The technology disclosed herein complies with legal and best practice privacy standards, such as HIPAA and GDPR.

At operation, the process may include generating a number of identifiers using a second hash function that is different from the first hash function. In one or more examples, individual identifiers may correspond to one or more tokens related to a respective individual of the group of individuals. The identifiers may be unique with respect to a given individual of the group of individuals and are de-identified. Additionally, the identifiers may be generated using information stored by the genomics data repository for the group of individuals that is different from the information stored by the genomics data repository used to generate the tokens. In various examples, intermediate identifiers may be generated by applying the second hash function to information of the respective groups of individuals and final versions of the identifiers may be generated by applying one or more salting techniques to the intermediate identifiers. Information stored by the genomics data repository for respective individuals may be stored in association with the identifiers such that at least a portion of the information for given individuals stored by the genomics data repository may be accessed using respective identifiers of the given individuals.

Further, the process may include, at operation, obtaining, using the number of identifiers, second data from the molecular data repository for the group of individuals, and, at operation, the process may include determining respective portions of the first data that correspond to respective portions of the second data for the group of individuals. For example, for a given individual, the first data corresponding to electronic medical record, including for example, patient information, health insurance claims, lifestyle data, for the given individual may be identified in addition to second data corresponding to molecular data of the given individual, such as genomics data. In this way, for a given individual, both electronic medical record, including for example, patient information, health insurance claims, lifestyle data, and molecular data may be identified.

The process may include, at operation, generating an integrated data repository that stores the respective portions of the first data and the respective portions of the second data in relation to respective identifiers of the number of identifiers. For example, the integrated data repository may store electronic medical record, including for example, patient information, health insurance claims, lifestyle data, and genomics claims data for a given individual in association with an identifier that may be used to access the electronic medical record, including for example, patient information, health insurance claims, lifestyle data, and the genomics claims data for the given individual. The information stored by the integrated data repository may be organized according to a data repository schema. For example, the integrated data repository may store electronic medical record, including for example, patient information, health insurance claims, lifestyle data, and genomics data for the group of individuals in a number of data tables. In one or more examples, information stored by the number of data tables may be linked. To illustrate, information related to a given individual stored by a first data table of the data repository schema may be linked to additional information related to the given individual stored by a second data table of the data repository schema. In this way, information accessed in one data table of the data repository schema may result in accessing additional information stored in another data table of the data repository schema.

In one or more illustrative examples, the data repository schema may include a first data table that stores genomics data of the group of individuals. For example, the first data table may store information corresponding to a panel used to generate genomics data, mutations of genomic regions, types of mutations, copy numbers of genomic regions, coverage data indicating numbers of nucleic acid molecules identified in a sample having one or more mutations, testing dates, and patient information. The data repository schema may also include a second data that stores data related to one or more patient visits by individuals to one or more healthcare providers and a third data table that stores information corresponding to respective services provided to individuals with respect to one or more patient visits to one or more healthcare providers indicated by the second data table. Additionally, the data repository schema may include a fourth data table that stores personal information of the group of individuals and a fifth data table that stores information related to a health insurance company or governmental entity that made payment for services provided to the group of individuals. Further, the data repository schema may include a sixth data table storing information corresponding to health insurance coverage information for the group of individuals, such as a type of health insurance plan related to the group of individuals. The data repository schema may also include a seventh data table that stores information related to pharmaceutical treatments obtained by the group of individuals.

In one or more examples, the integrated data repository may also store medical records that correspond to at least a portion of the group of individuals. In these examples, the medical records may be obtained from one or more data repositories storing the medical records. One or more optical character recognition (OCR) operations may be performed with respect to the medical records. Additionally, the medical records may be analyzed to determine one or more portions of the additional information to remove to produce a corpus of information. In various examples, the corpus of information may be analyzed to determine a portion of the subset of the additional group of individuals that correspond to one or more biomarkers.

One or more data structures may be generated from the corpus of information that store identifiers of the portion of the subset of the additional group of individuals and that store an indication that the portion of the subset of the additional group of individuals corresponds to the one or more biomarkers. The one or more data structures may be stored by an intermediate data repository. One or more de-identification operations may be performed with respect to the identifiers of the portion of the subset of the additional group of individuals before modifying the integrated data repository to store at least a portion of the additional information of the medical records of the portion of the subset of the additional group of individuals in relation to the number of identifiers. After de-identification of the information stored by the one or more data structures, the information stored by the integrated data repository may be added to the integrated data repository. In at least some examples, the de-identified medical records information may be added to the integrated data repository in addition to or in lieu of the electronic medical record, including for example, patient information, health insurance claims, lifestyle data. In various examples, the one or more data structures storing the de-identified medical records information with respect to the biomarker data may have one or more logical connections with other data structures stored in the integrated data repository. To illustrate, the one or more data structures storing the de-identified medical records information with respect to the biomarker data may have one or more logical connections with at least one of the first data table may store information corresponding to a panel used to generate genomics data, mutations of genomic regions, types of mutations, copy numbers of genomic regions, coverage data indicating numbers of nucleic acid molecules identified in a sample having one or more mutations, testing dates, and patient information, the second data that stores data related to one or more patient visits by individuals to one or more healthcare providers, the a third data table that stores information corresponding to respective services provided to individuals with respect to one or more patient visits to one or more healthcare providers indicated by the second data table, the fourth data table that stores personal information of the group of individuals, the fifth data table that stores information related to a health insurance company or governmental entity that made payment for services provided to the group of individuals, the sixth data table storing information corresponding to health insurance coverage information for the group of individuals, such as a type of health insurance plan related to the group of individuals, or the seventh data table that stores information related to pharmaceutical treatments obtained by the group of individuals.

In various examples, the medical records data may be added to the integrated data repository by generating a data file including the first tokens generated using a first hash function. Individual first tokens may correspond to a respective individual of a group of individuals having data stored by a molecular data repository. Additionally, the data file may be sent to a medical records data management system and medical records data corresponding to the group of individuals may be obtained from the medical records data management system in response to the data file. Further, a number of identifiers may be generated using a second hash function that is different from the first hash function. Each identifier may correspond to one or more tokens related to each individual of the group of individuals. Using the number of identifiers second data may be obtained from the molecular data repository for the group of individuals. In various examples, respective portions of the first data may be determined to correspond to respective portions of the second data for the group of individuals. In this way, the integrated data repository may be generated that stores the respective portions of the first data and the respective portions of the second data in relation to respective identifiers of the number of identifiers.

After the integrated data repository storing medical records data is generated, a request may be received to determine data with respect to a number of individuals having data stored in the integrated data repository. The request includes one or more search criteria. In one or more examples, a subset of the number of individuals having one or more characteristics that correspond to the one or more search criteria may be determined and information of the subset of the number of individuals may be analyzed to determine a measure of significance of a characteristic of the one or more characteristics with respect to a biological condition.

In one or more illustrative examples, one or more genomic mutations may be determined to be present in the subset of the number of individuals and a plurality of treatments provided to the subset of the number of individuals may also be determined. In various examples, respective survival rates for the subset of the number of individuals may be determined, such as real-world survival rates. In at least some examples, the measure of significance may correspond to survival rate with respect to a treatment of the plurality of treatments and a genomic mutation of the one or more genomic mutations. Based on measure of significance, an effectiveness of the treatment for the subset of the number of individuals may be determined. In one or more examples, individuals in subset of the number of individuals that have not received the treatment may be determined. One or more therapeutically effective amounts of the treatment may be administered to the individuals in the subset of the number of individuals that have not received the treatment.

Described herein is a data flow diagram of an example process to generate a number of datasets used to analyze information stored by an integrated data repository that stores electronic medical record, including for example, patient information, health insurance claims, lifestyle data, and genomics data, according to one or more implementations. The process may include, at operation, determining a first set of data processing instructions that are executable in relation to the first data stored by an integrated data repository. The integrated data repository may store electronic medical record, including for example, patient information, health insurance claims, lifestyle data, and molecular data for a common group of individuals. In one or more examples, the first set of data processing instructions may be included in a plurality of sets of data processing instructions that are part of a data processing pipeline. Each of the sets of data processing instructions of the data processing pipeline may be executed to generate a respective analytics ready dataset. For example, individual sets of data processing instructions of the data processing pipeline may be executable to generate datasets that include specified portions of information and/or combinations of information stored by the integrated data repository. In one or more additional examples, individual sets of data processing instructions of the data processing pipeline may be executable to analyze and modify portions of information stored by the integrated data repository to generate respective datasets. Additionally, individual sets of data processing instructions may be executable with respect to individual subsets of information stored by the integrated data repository.

The process may also include, at operation, causing the first set of data processing instructions to be executed to generate a first dataset. The first dataset may indicate a subset of the group of individuals in which a biological condition is present. The first set of data processing instructions may be executed to analyze data stored by the integrated data repository to identify a cohort of individuals in which the biological condition is present. In one or more illustrative examples, the biological condition may include a cancer. To illustrate, the first set of data processing instructions may be executed to analyze data stored by the integrated data repository to identify a cohort of individuals in which lung cancer is present. In various examples, the data processing pipeline may include multiple sets of data processing instructions to identify cohorts of individuals in which different biological conditions are present.

In one or more examples, the first set of data processing instructions may be executed to analyze at least one of electronic medical record, including for example, patient information, health insurance claims, lifestyle data, or molecular data to determine a cohort of individuals in which the biological condition is present. For example, the first set of data processing instructions may be executed to identify individuals having one or more health insurance codes present in electronic medical record, including for example, patient information, health insurance claims, lifestyle data, to determine a group of individuals in which the biological condition is present. Additionally, the first set of data processing instructions may be executed to identify individuals in which one or more mutations are present in a genomic region of nucleic acid molecules derived from samples obtained from the individuals to determine a group of individuals in which the biological condition is present.

In addition, the process may include, at operation, determining a second set of data processing instructions that are executable in relation to second data stored by the integrated data repository. The second set of data stored by the integrated data repository may be different from the first set of data stored by the integrated data repository and analyzed in relation to the first set of data processing instructions. For example, the first data may correspond to first columns of one or more first data tables stored by the integrated data repository and the second data may correspond to second columns of one or more second data tables stored by the integrated data repository.

At operation, the process may include causing the second set of data processing instructions to be executed to generate a second dataset indicating one or more treatments provided to a second subset of the group of individuals. The second dataset may indicate a subset of the group of individuals that have received one or more treatments. The one or more treatments may be provided to individuals in which one or more biological conditions are present. In one or more examples, the second set of data processing instructions may be executed to analyze data stored by the integrated data repository to identify a cohort of individuals that received the one or more treatments. To illustrate, the second set of data processing instructions may be executed to analyze at least one electronic medical record, including for example, patient information, health insurance claims, lifestyle data, or genomics data to determine a cohort of individuals that received the one or more treatments. In one or more illustrative examples, the second set of data processing instructions may be executed to identify individuals having one or more health insurance codes present in electronic medical record, including for example, patient information, health insurance claims, lifestyle data, to determine a group of individuals that received the one or more treatments.

Further, the process may include, at operation, determining a third subset of the group of individuals that includes a portion of the first subset of the group of individuals that overlaps with a portion of the second subset of the group of individuals. As a result, the third subset of the group of individuals corresponds to individuals in which both the biological condition is present and the one or more treatments are provided. At, the process, may include analyzing the first dataset and the second dataset with respect to the third subset of the group of individuals to determine a measure of significance of a characteristic of the third subset of the group of individuals. In one or more examples, one or more machine learning techniques or statistical techniques may be applied to information included in at least one of the first dataset and the second dataset with respect to the third subset of the group of individuals. The measure of significance may correspond to a statistical measure of significance with respect to the characteristic. In one or more additional examples, the measure of significance may correspond to a probability of the characteristic being present in individuals in which the biological condition is present.

In one or more illustrative examples, the characteristic may include one or more treatments provided to the individuals in which the biological condition is present. In one or more additional illustrative examples, the characteristic may include the presence of a mutation of a genomic region of nucleic acid molecules derived from samples obtained from individuals in which the biological condition is present. In various examples, information included in at least one of the first dataset or the second dataset may be analyzed to determine an impact of the characteristic with respect to one or more metrics. In one or more examples, information included in at least one of the first dataset or the second dataset may be analyzed to determine an amount of influence of a treatment on a survival rate of individuals in which the biological condition is present. In one or more further examples, information included in at least one of the first dataset or the second dataset may be analyzed to determine an amount of influence of a mutation of a genomic region on a survival rate of individuals in which the biological condition is present. Additionally, information included in the first dataset and the second dataset may be analyzed to determine an amount of impact of one or more treatments with respect to individuals in which the biological condition is present and in which one or more genomic mutations are also present.

Described herein is a machine in the form of a computer system within which a set of instructions may be executed for causing the machine to perform any one or more of the methodologies discussed herein, according to an example, according to an example implementation. For example, a machine in the example form of a computer system, within which instructions (e.g., software, a program, an application, an applet, an app, or other executable code) for causing the machine to perform any one or more of the methodologies discussed herein may be executed. For example, the instructions may cause the machine to implement the architectures and frameworks described previously, and to execute the methods described with respect to previously.

The instructions transform the general, non-programmed machine into a particular machine programmed to carry out the described and illustrated functions in the manner described. In alternative implementations, the machine operates as a standalone device or may be coupled (e.g., networked) to other machines. In a networked deployment, the machine may operate in the capacity of a server machine or a client machine in a server-client network environment, or as a peer machine in a peer-to-peer (or distributed) network environment. The machine may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions, sequentially or otherwise, that specify actions to be taken by the machine. Further, while only a single machine is illustrated, the term “machine” shall also be taken to include a collection of machines that individually or jointly execute the instructions to perform any one or more of the methodologies discussed herein.

Examples of computing devices may include logic, one or more components, circuits (e.g., modules), or mechanisms. Circuits are tangible entities configured to perform certain operations. In an example, circuits may be arranged (e.g., internally or with respect to external entities such as other circuits) in a specified manner. In an example, one or more computer systems (e.g., a standalone, client or server computer system) or one or more hardware processors (processors) may be configured by software (e.g., instructions, an application portion, or an application) as a circuit that operates to perform certain operations as described herein. In an example, the software may reside (1) on a non-transitory machine readable medium or (2) in a transmission signal. In an example, the software, when executed by the underlying hardware of the circuit, causes the circuit to perform the certain operations.

In an example, a circuit may be implemented mechanically or electronically. For example, a circuit may comprise dedicated circuitry or logic that is specifically configured to perform one or more techniques such as discussed above, such as including a special-purpose processor, a field programmable gate array (FPGA) or an application-specific integrated circuit (ASIC). In an example, a circuit may comprise programmable logic (e.g., circuitry, as encompassed within a general-purpose processor or other programmable processor) that may be temporarily configured (e.g., by software) to perform the certain operations. It will be appreciated that the decision to implement a circuit mechanically (e.g., in dedicated and permanently configured circuitry), or in temporarily configured circuitry (e.g., configured by software) may be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise, a general-purpose processor configured via software, the general-purpose processor may be configured as respective different circuits at different times. Software may accordingly configure a processor, for example, to constitute a particular circuit at one instance of time and to constitute a different circuit at a different instance of time.

In an example, circuits may provide information to, and receive information from, other circuits. In this example, the circuits may be regarded as being communicatively coupled to one or more other circuits. Where multiples of such circuits exist contemporaneously, communications may be achieved through signal transmission (e.g., over appropriate circuits and buses) that connect the circuits. In implementations in which multiple circuits are configured or instantiated at different times, communications between such circuits may be achieved, for example, through the storage and retrieval of information in memory structures to which the multiple circuits have access. For example, one circuit may perform an operation and store the output of that operation in a memory device to which it is communicatively coupled. A further circuit may then, at a later time, access the memory device to retrieve and process the stored output. In an example, circuits may be configured to initiate or receive communications with input or output devices and may operate on a resource (e.g., a collection of information).

The various operations of method examples described herein may be performed, at least partially, by one or more processors that are temporarily configured (e.g., by software) or permanently configured to perform the relevant operations. Whether temporarily or permanently configured, such processors may constitute processor-implemented circuits that operate to perform one or more operations or functions. In an example, the circuits referred to herein may comprise processor-implemented circuits.

Similarly, the methods described herein may be at least partially processor implemented. For example, at least some or all of the operations of a method may be performed by one or processors or processor-implemented circuits. The performance of certain of the operations may be distributed among the one or more processors, not only residing within a single machine, but deployed across a number of machines. In an example, the processor or processors may be located in a single location (e.g., within a home environment, an office environment or as a server farm), while in other examples the processors may be distributed across a number of locations.

The one or more processors may also operate to support performance of the relevant operations in a “cloud computing” environment or as a “software as a service.”

(SaaS). For example, at least some of the operations may be performed by a group of computers (as examples of machines including processors), with these operations being accessible via a network (e.g., the Internet) and via one or more appropriate interfaces (e.g., Application Program Interfaces (APIs).)

Example implementations (e.g., apparatus, systems, or methods) may be implemented in digital electronic circuitry, in computer hardware, in firmware, in software, or in any combination thereof. Example implementations may be implemented using a computer program product (e.g., a computer program, tangibly embodied in an information carrier or in a machine readable medium, for execution by, or to control the operation of, data processing apparatus such as a programmable processor, a computer, or multiple computers).

A computer program may be written in any form of programming language, including compiled or interpreted languages, and it may be deployed in any form, including as a stand-alone program or as a software module, subroutine, or other unit suitable for use in a computing environment. A computer program may be deployed to be executed on one computer or on multiple computers at one site or distributed across multiple sites and interconnected by a communication network.

In an example, operations may be performed by one or more programmable processors executing a computer program to perform functions by operating on input data and generating output. Examples of method operations may also be performed by, and example apparatus may be implemented as, special purpose logic circuitry (e.g., a field programmable gate array (FPGA) or an application-specific integrated circuit (ASIC)).

The computing system may include clients and servers. A client and server are generally remote from each other and generally interact through a communication network. The relationship of client and server arises by virtue of computer programs running on the respective computers and having a client-server relationship with each other. In implementations deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware may be a design choice. Below are set out hardware (e.g., computing device) and software architectures that may be deployed in example implementations.

In an example, the computing device may operate as a standalone device or the computing device may be connected (e.g., networked) to other machines.

In a networked deployment, the computing device may operate in the capacity of either a server or a client machine in server-client network environments. In an example, computing device may act as a peer machine in peer-to-peer (or other distributed) network environments. The computing device may be a personal computer (PC), a tablet PC, a set-top box (STB), a mobile telephone, a web appliance, a network router, switch or bridge, or any machine capable of executing instructions (sequential or otherwise) specifying actions to be taken (e.g., performed) by the computing device. Further, while only a single computing device is illustrated, the term “computing device” shall also be taken to include any collection of machines that individually or jointly execute a set (or multiple sets) of instructions to perform any one or more of the methodologies discussed herein.

Example computing device may include a processor (e.g., a central processing unit CPU), a graphics processing unit (GPU) or both), a main memory and a static memory, some or all of which may communicate with each other via a bus. The computing device may further include a display unit, an alphanumeric input device (e.g., a keyboard), and a user interface (UI) navigation device (e.g., a mouse). In an example, the display unit, input device and UI navigation device may be a touch screen display. The computing device may additionally include a storage device (e.g., drive unit), a signal generation device (e.g., a speaker), a network interface device, and one or more sensors, such as a global positioning system (GPS) sensor, compass, accelerometer, or another sensor.

The storage device may include a machine readable medium on which is stored one or more sets of data structures or instructions (e.g., software) embodying or utilized by any one or more of the methodologies or functions described herein. The instructions may also reside, completely or at least partially, within the main memory, within static memory, or within the processor during execution thereof by the computing device. In an example, one or any combination of the processor, the main memory, the static memory, or the storage device may constitute machine readable media.

While the machine readable medium is illustrated as a single medium, the term “machine readable medium” may include a single medium or multiple media (e.g., a centralized or distributed database, and/or associated caches and servers) that configured to store the one or more instructions. The term “machine readable medium” may also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” may accordingly be taken to include, but not be limited to, solid-state memories, and optical and magnetic media. Specific examples of machine-readable media may include non-volatile memory, including, by way of example, semiconductor memory devices (e.g., Electrically Programmable Read-Only Memory

(EPROM), Electrically Erasable Programmable Read-Only Memory (EEPROM) and flash memory devices; magnetic disks such as internal hard disks and removable disks; magneto-optical disks; and CD-ROM and DVD-ROM disks.

The instructions may further be transmitted or received over a communications network 828 using a transmission medium via the network interface device 822 utilizing any one of a number of transfer protocols (e.g., frame relay, IP, TCP, UDP, HTTP, etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

As used herein, a component, may refer to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example implementations, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

### Cancer and Other Diseases

The present methods can be used to diagnose presence of conditions, particularly cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of copy number variation or rare mutations detected in subject's blood if the treatment is successful as more cancers may die and shed DNA. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor residual disease or recurrence of disease.

In some embodiments, the methods and systems disclosed herein may be used to identify customized or targeted therapies to treat a given disease or condition in patients based on the classification of a nucleic acid variant as being of somatic or germline origin. Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile includes a plurality of data resulting from copy number variation and rare mutation analyses. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate our profile, fingerprint or set of data that is a summation of genetic information derived from different cells in a heterogeneous disease. This set of data may comprise copy number variation, epigenetic variation, and mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardiofacial syndrome, WAGR syndrome, Wilson disease, or the like.

In some embodiments, a method described herein includes detecting a presence or absence of DNA originating or derived from a tumor cell at a preselected timepoint following a previous cancer treatment of a subject previously diagnosed with cancer using a set of sequence information obtained as described herein. The method may further comprise determining a cancer recurrence score that is indicative of the presence or absence of the DNA originating or derived from the tumor cell for the test subject. Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the test subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The methods discussed above may further comprise any compatible feature or features set forth elsewhere herein, including in the section regarding methods of determining a risk of cancer recurrence in a test subject and/or classifying a test subject as being a candidate for a subsequent cancer treatment.

Methods of Determining a Risk of Cancer Recurrence in a Test Subject and/or Classifying a Test Subject as Being a Candidate for a Subsequent Cancer Treatment.

In some embodiments, a method provided herein is a method of determining a risk of cancer recurrence in a test subject. In some embodiments, a method provided herein is a method of classifying a test subject as being a candidate for a subsequent cancer treatment.

Any of such methods may comprise collecting DNA (e.g., originating or derived from a tumor cell) from the test subject diagnosed with the cancer at one or more preselected timepoints following one or more previous cancer treatments to the test subject. The subject may be any of the subjects described herein. The DNA may be cfDNA. The DNA may be obtained from a tissue sample.

Any of such methods may comprise capturing a plurality of sets of target regions from DNA from the subject, wherein the plurality of target region sets includes a sequence-variable target region set and an epigenetic target region set, whereby a captured set of DNA molecules is produced. The capturing step may be performed according to any of the embodiments described elsewhere herein. In any of such methods, the previous cancer treatment may comprise surgery, administration of a therapeutic composition, and/or chemotherapy.

Any of such methods may comprise sequencing the captured DNA molecules, whereby a set of sequence information is produced. The captured DNA molecules of the sequence-variable target region set may be sequenced to a greater depth of sequencing than the captured DNA molecules of the epigenetic target region set.

Any of such methods may comprise detecting a presence or absence of DNA originating or derived from a tumor cell at a preselected timepoint using the set of sequence information. The detection of the presence or absence of DNA originating or derived from a tumor cell may be performed according to any of the embodiments thereof described elsewhere herein.

Methods of determining a risk of cancer recurrence in a test subject may comprise determining a cancer recurrence score that is indicative of the presence or absence, or amount, of the DNA originating or derived from the tumor cell for the test subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of classifying a test subject as being a candidate for a subsequent cancer treatment may comprise comparing the cancer recurrence score of the test subject with a predetermined cancer recurrence threshold, thereby classifying the test subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment includes chemotherapy or administration of a therapeutic composition.

Any of such methods may comprise determining a disease-free survival (DFS) period for the test subject based on the cancer recurrence score; for example, the DFS period may be 1 year, 2 years, 3, years, 4 years, 5 years, or 10 years.

In some embodiments, the set of sequence information includes sequence-variable target region sequences, and determining the cancer recurrence score may comprise determining at least a first subscore indicative of the amount of SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, the set of sequence information includes epigenetic target region sequences, and determining the cancer recurrence score includes determining a second subscore indicative of the amount of molecules (obtained from the epigenetic target region sequences) that represent an epigenetic state different from DNA found in a corresponding sample from a healthy subject (e.g., cfDNA found in a blood sample from a healthy subject, or DNA found in a tissue sample from a healthy subject where the tissue sample is of the same type of tissue as was obtained from the test subject). These abnormal molecules (i.e., molecules with an epigenetic state different from DNA found in a corresponding sample from a healthy subject) may be consistent with epigenetic changes associated with cancer, e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the second subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the epigenetic target regions, e.g., including one or both of hypermethylation variable target regions and fragmentation variable target regions (hypermethylation of a hypermethylation variable target region and/or abnormal fragmentation of a fragmentation variable target region may be considered indicative of origination from a tumor cell). This may be done for molecules corresponding to sequence variable target regions, e.g., molecules including alterations consistent with cancer, such as SNVs, indels, CNVs, and/or fusions. The fraction of tumor DNA may be determined based on a combination of molecules corresponding to epigenetic target regions and molecules corresponding to sequence variable target regions.

Determination of a cancer recurrence score may be based at least in part on the fraction of tumor DNA, wherein a fraction of tumor DNA greater than a threshold in the range of 10-11 to 1 or 10-10 to 1 is sufficient for the cancer recurrence score to be classified as positive for cancer recurrence. In some embodiments, a fraction of tumor DNA greater than or equal to a threshold in the range of 10-10 to 10-9, 10-9 to 10-8, 10-8 to 10-7, 10-7 to 10-6, 10-6 to 10-5, 10-5 to 10-4, 10-4 to 10-3, 10-3 to 10-2, or 10-2 to 10-1 is sufficient for the cancer recurrence score to be classified as positive for cancer recurrence. In some embodiments, the fraction of tumor DNA greater than a threshold of at least 10-7 is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information includes sequence-variable target region sequences and epigenetic target region sequences, and determining the cancer recurrence score includes determining a first subscore indicative of the amount of SNVs, insertions/deletions, CNVs and/or fusions present in sequence-variable target region sequences and a second subscore indicative of the amount of abnormal molecules in epigenetic target region sequences, and combining the first and second subscores to provide the cancer recurrence score. Where the first and second subscores are combined, they may be combined by applying a threshold to each subscore independently (e.g., greater than a predetermined number of mutations (e.g., >1) in sequence-variable target regions, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### Therapies and Related Administration

In certain embodiments, the methods disclosed herein relate to identifying and administering customized therapies to patients given the status of a nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the test subject and/or patients who are receiving, or who have received, the same therapy as the test subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it should be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

### Kits

Also provided are kits including the compositions as described herein. The kits can be useful in performing the methods as described herein. In some embodiments, a kit includes a first reagent for partitioning a sample into a plurality of subsamples as described herein, such as any of the partitioning reagents described elsewhere herein. In some embodiments, a kit includes a second reagent for subjecting the first subsample to a procedure that affects a first nucleobase in the DNA differently from a second nucleobase in the DNA of the first subsample, wherein the first nucleobase is a modified or unmodified nucleobase, the second nucleobase is a modified or unmodified nucleobase different from the first nucleobase, and the first nucleobase and the second nucleobase have the same base pairing specificity (e.g., any of the reagents described elsewhere herein for converting a nucleobase such as cytosine or methylated cytosine to a different nucleobase). The kit may comprise the first and second reagents and additional elements as discussed below and/or elsewhere herein.

Kits may further comprise a plurality of oligonucleotide probes that selectively hybridize to least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCHI, NRAS, PIK3CA, PTEN, RBI, TP53, MET, AR, ABL1, AKT1, ATM, CDH1, CSFIR, CTNNB1, ERBB4, EZH2, FGFR1, FGFR2, FGFR3, FLT3, GNA11, GNAQ, GNAS, HNF1A, HRAS, IDH1, IDH2, JAK2, JAK3, KDR, KIT, MLH1, MPL, NPM1, PDGFRA, PROC, PTPN11, RET, SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RIT1, GATA3, MAP2K1, RHEB, ROS1, ARAF, MAP2K2, NFE2L2, RHOA, and NTRK1. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The oligonucleotide probes can selectively hybridize to exon regions of the genes, e.g., of the at least 5 genes. In some cases, the oligonucleotide probes can selectively hybridize to at least 30 exons of the genes, e.g., of the at least 5 genes. In some cases, the multiple probes can selectively hybridize to each of the at least 30 exons. The probes that hybridize to each exon can have sequences that overlap with at least 1 other probe. In some embodiments, the oligoprobes can selectively hybridize to non-coding regions of genes disclosed herein, for example, intronic regions of the genes. The oligoprobes can also selectively hybridize to regions of genes including both exonic and intronic regions of the genes disclosed herein.

Any number of exons can be targeted by the oligonucleotide probes. For example, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 400, 500, 600, 700, 800, 900, 1,000, or more, exons can be targeted.

The kit can comprise at least 4, 5, 6, 7, or 8 different library adaptors having distinct molecular barcodes and identical sample barcodes. The library adaptors may not be sequencing adaptors. For example, the library adaptors do not include flow cell sequences or sequences that permit the formation of hairpin loops for sequencing. The different variations and combinations of molecular barcodes and sample barcodes are described throughout and are applicable to the kit. Further, in some cases, the adaptors are not sequencing adaptors. Additionally, the adaptors provided with the kit can also comprise sequencing adaptors. A sequencing adaptor can comprise a sequence hybridizing to one or more sequencing primers. A sequencing adaptor can further comprise a sequence hybridizing to a solid support, e.g., a flow cell sequence. For example, a sequencing adaptor can be a flow cell adaptor. The sequencing adaptors can be attached to one or both ends of a polynucleotide fragment. In some cases, the kit can comprise at least 8 different library adaptors having distinct molecular barcodes and identical sample barcodes. The library adaptors may not be sequencing adaptors. The kit can further include a sequencing adaptor having a first sequence that selectively hybridizes to the library adaptors and a second sequence that selectively hybridizes to a flow cell sequence. In another example, a sequencing adaptor can be hairpin shaped. For example, the hairpin shaped adaptor can comprise a complementary double stranded portion and a loop portion, where the double stranded portion can be attached {e.g., ligated) to a double-stranded polynucleotide. Hairpin shaped sequencing adaptors can be attached to both ends of a polynucleotide fragment to generate a circular molecule, which can be sequenced multiple times. A sequencing adaptor can be up to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adaptor can comprise 20-30, 20-40, 30-50, 30-60, 40-60, 40-70, 50-60, 50-70, bases from end to end. In a particular example, the sequencing adaptor can comprise 20-30 bases from end to end. In another example, the sequencing adaptor can comprise 50-60 bases from end to end. A sequencing adaptor can comprise one or more barcodes. For example, a sequencing adaptor can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 21, 22, 23, 24, 25, or more (or any length as described throughout) nucleic acid bases, e.g., at least 8 bases. The barcode can be contiguous or non-contiguous sequences, as described above.

The library adaptors can be blunt ended and Y-shaped and can be less than or equal to 40 nucleic acid bases in length. Other variations of the can be found throughout and are applicable to the kit.

### Biomarkers

The disclosure provides methods of using biomarkers for the diagnosis, prognosis, and therapy selection of a subject suffering from e.g., cancer. A biomarker may be any gene or variant of a gene whose presence, mutation, deletion, substitution, copy number, or translation (i.e., to a protein) is an indicator of a disease state. Biomarkers of the present disclosure may include the presence, mutation, deletion, substitution, copy number, or translation. Examples include the aforementioned in any one or more of EGFR, KRAS, MET, BRAF, MYC, NRAS, ERBB2, ALK, Notch, PIK3CA, APC, and SMO.

Example related to MTAP include one or more of: CDKN2A-DT, ELAVL2, THBD, CYP27C1, SH3BP2, TXNRD1, ESYT3, LOC105378933, MN1, HTRA3, IDH2, IDH2-DT, ELFN2, ACTC1, PTGS2, PACERR, WWC2, WWC2-AS2, OSMR-AS1, OSMR, CHST15, COL18A1, TGFBI, SHISA2, HACD4, PRKG1-AS1, DKK1, CCK, CLIP4, HIF3A, GPR156, MIR6068, CDKN2B-AS1, CDKN2A, VLDLR-AS1, PTHLH, H2BP2, PAK5, COL4A3, COL4A4, BMPR1B-DT, BMPR1B, PCDHGC3, PLA2R1, RBPMS-AS1, CAV2, RASL12, PPFIA2, TIAM1, PAPLN, MIR31HG, LOXL2, LRRC71, MAMDC2, CSGALNACT1, GLP1R, PDGFD, EPB41L4A, EPB41L4A-DT, TMEM200B, CYP19A1, GLDN, SLC9A7P1, NXNL2, LOC100130207, IER3, FLOT1, IER3-AS1, C7orf57, GPR12, CXCL1, SPAG17, BEGAIN, HSD52, UHRF2, ERC2, NT5E, MAP10, MIR4785, LINC01574, DMRTA1, AKR1B1, SPOCK3, CASR, ALKALI, FMN1, CHST11, STK32A, STK32A-AS1, LOC100507144, CD44, AFAP1L2, ICAM1, RAD52, WIPF1, EMBP1, SLC6A20, RBP7, CHRNB4, KCNS2, DOCK8, GALNT16, CPNE8, DUOXA1, DUOX1, FRMD5, FAM217B, SYCP2, FLNC, SCHIP1, RPL39L, LINC02609, LINC01763, and/or CD40.

Additional examples related to MTAP include one or more of TRIM67, PCDH8, OLIG2, AP000282.2, RP11-65J21.3, MIR193B, RP11-65J21.3, LTBP2, NKX2-1-AS1, GLYATL2, GLYATL1, RP11-522I20.3, UBQLN1, RP11-147L13.2, AMZ2, SQSTM1, MGAT4B, PRR14, ADAMTS20, FOXG1-AS1, FOXG1, TCTEX1D1, EVL, TOR1B, BARHL2, OSTM1, NR2E1, ACSF2, MIR193A, RP1-41C23.4, CCDC178, ABCC9, CACNG8, ANKRD20A8P, NRN1, DNMT3A, ITGA8, RP11-501C14.5, IGF2BP1, RP11-501C14.5, ELMO1, MICU3, FOXF2, ZNF775, TGFBR1, RAG1, TRAF6, RP11-545D22.1, ZNF607, CTD-2528L19.4, RP11-742D12.2, ST8SIA5, OTX2, ATG12, AP3S1, AJ003147.9, RP11-816J6.3, ACTRT3, TET2, BVES, BVES-AS1, NT5E, PCDHGA10, PCDHGB1, PCDHGB3, PCDHGB2, PCDHGA8, PCDHGA9, PCDHGB7, PCDHGB6, PCDHGB5, PCDHGB4, PCDHGA2, PCDHGA3, PCDHGA1, PCDHGA6, PCDHGA7, PCDHGA4, PCDHGA5, CNPY1, AC008060.5, CCDC140, PAX3, ZNF667-AS1, ZNF667, TMEM155, PP12613, CTC-512J12.4, CTC-512J12.6, ZNF285, EPB41L3, GSX1, CDH3, SMC5-AS1, SMC5, ELAVL4, PCOLCE2, CCDC140, PAX3, SPAG6, PDCL, HNF1B, ZNF790-AS1, SORCS3, PNMA1, PRMT3, FAM183A, DLX2-AS1, DLX2, EPB41L3, SOX11, LINC01248, AC108025.2, SOX11, LINC01248, SOX11, HOXA11-AS, RP1-170019.14, ALDH7A1, GPR158-AS1, GPR158, RAI1, AC093887.1, POU4F2, GALT, RP11-195F19.29, AC008753.6, MYADM, ST20-AS1, ST20, ST20-MTHFS, RP11-81A1.6, ST20, ST20-MTHFS, ST20-AS1, KLHL9, RP11-744N12.3, FLI1, RYR1, KCNIP2, SLC6A2, LAMA1, GSG1L, CUX2, RXRG, SH3GL3, KIRREL, NCR3LG1, AC093627.12, and/or FAM20C.

Other examples related to PTEN include one or more of: HOXC-AS3, FGF8, TJP1, RHBDD1, IRS1, ATP10A, HTR1B, BASP1, CHMP4A, MEIS1, CH25H, SYT5, HOXB5, HOXC-AS3, CDHR5, SCT, MIR6719, DYRKIB, PAH, IL12RB2, PMP22, H2BC3, FOXCUT, FOXC1, CXXC5, PAM, PCDHAC1, GATA4, OSR1, MIR193B, MIR193BHG, RUSC1, PIK3R3, SESN3, CACNAlC-AS1, HOXB-AS3, GHRL, LINC02560, STK3, OSR2, ZNF565, RHBDD1, BHLHE40, BHLHE40-AS1, CAST, ACTC1, OSBP2, RECQL4, HMGA1, MIR6835, EYA1, LOC101929340, SHISA8, H3C8, H2BC10, LINC01006, PRDM16, PRDM16-DT, LINC01833, ANKRD45, LOC105379152, ADHFE1, RRS1-AS1, H3C7, H4C7, E2F8, TBX1, TBX15, HPN, CALHM2, NPPB, LBX2, DERL3, NKX3-2, HOXB5, PHPT1, KCNJ3, HSF4, TRADD, RPP25, SIM2, STX16-NPEPL1, STX16, PTTG1IP, ZNF292, SPEG, FES, PKP1, LHX1, HOXB3, PRDM16-DT, MMP23B, AGMAT, DUOX2, NKX2-2, TBX6, LOC101928595, MVP, PAGR1, HEBP1, CRYM, NTRK2, LOC145845, ATP13A3, TJP1, SERHL, ARHGEF19, LOC100507634, MIR4422HG, USP24, RASSF1, RASSF1-AS1, RHOH, BARX2, FILIPIL, CMSS1, ZNF546, RARA-AS1, USP3, CBX4, SPATA18, ODAD2P1, SETBP1, NUTF2, THAP11, C6orf141, RND2, VAT1, and/or CAMTA2.

Additional examples related to PTEN include one or more of: SGIP1, SYT11, GON4L, STC2, STC2, MTUS2, STK32B, FBXL2, SYK, GBX2, AC079135.1, LMO1, ZNF423, POU4F1, RNF219-AS1, RIN3, HOXD11, HOXB8, HOXB7, AC005597.1, ZNF578, LINC01415, RP11-397A16.1, DPY19L2P1, C10orf54, CDH23, ZCCHC24, C2orf43, OLIG3, BARX1, BARX1-AS1, FAM149A, FAM69B, PCDHA2, PCDHA3, PCDHA1, PCDHA6, PCDHA7, PCDHA4, PCDHA5, CTC-270D5.2, PCDHA9, PCDHAC2, PCDHAC1, PCDHA10, PCDHA11, PCDHA12, PCDHA13, PCDHA8, LRRC32, ST8SIA2, TUB, ROBO3, KIAA0319, SSPN, TBX1, NTF3, FBLL1, RGS17, CTNNA2, KCND2, TNS3, HIF3A, UPP1, ABR, C1QTNF1, C1QTNF1-AS1, FAM19A5, KCTD2, FENDRR, FOXF1, FENDRR, MIR193A, RP1-41C23.4, PLCD1, ZKSCAN7, RP11-62H20.1, LRRC9, EIF1B, EIF1B-AS1, AJ003147.9, ATP10A, MCHR2-AS1, MCHR2, WNT9A, RP11-428C19.4, E2F8, FAM110C, PTEN, KLLN, GSDMD, FES, ADD2, ELMO1, BNIP3, RP11-114H24.4, MIAT, MIAT_exon1, NALCN, RNF219-AS1, RP11-52L5.6, NRN1, CAPN5, OAT, COCH, LPL, GLIS2, GLIS2-AS1, MOB3B, COLCA1, COLCA2, N4BP2L1, RP11-298P3.4, GS1-72M22.1, NEFM, PRMT8, FOXB2, STK25, RP11-2N1.2, CCBE1, ISL1, CTD-2314G24.2, SFRP1, PCDH18, ARMC3, ST6GAL2, WNK2, SLC35C2, RP11-39402.3, LEPR, ADAM19, NIPAL4, CTB-109A12.1, BBC3, MIR3190, TFAP2A, TFAP2A-AS1, RP11-470C13.4, INSM1, and/or LINC01096.

Other examples related to EGFR include one or more of: EGFR, CPZ, GPR78, TOX2, ADCY8, GAS7, TRIM71, SRRM4, RP4-563E14.1, DIDO1, VPS37B, P2RX2, NIM1K, ZNF131, NIM1K, OPRK1, CLDN10, STMN3, LINC00472, VIM-AS1, VIM, RP11-32K4.1, PVR, CTB-171A8.1, CACNA1G-AS1, CACNA1G, CCND2-AS1, CCND2-AS2, CCND2, CCND2-AS1, CCND2-AS2, RHEB, MIR6068, ANKRD63, PLCB2, FOXJ1, RP11-449J21.5, LHX5, GRID1, PAX7, LINC00925, PIK3R5, CTB-41I6.1, LIMS2, ZNF132, CTD-2619J13.19, PTPRO, RERG, WIF1, FAIM2, C5orf38, IRX2, SOX1, HSP90AA1, NACAD, ANK1, GPR158-AS1, GPR158, BEND6, DST, EFCC1, KIAA1257, SUN1, C9orf50, NTMT1, EYA2, ZSCAN31, KAZN, ADAMTS5, ZFP37, SETD1B, SETD1B, LINC01089, RHOF, SETD1B, LINC01089, RP11-467K18.2, NRIP3, ZFPM2, MIR193A, RP1-41C23.4, SHISA6, GLT1D1, RP11-672L10.2, RP11-672L10.3, ADCYAP1, RP11-379F12.4, GATA3-AS1, RP11-379F12.4, FGF14, RIMS4, RNF220, FES, ARRDC2, SEZ6L, USH1C, RAMP2-AS1, RAMP2, WNT2, ZIK1, TOX2, AAAS, ADAMTS18, RP11-449J10.1, RP11-713D19.1, SPDYA, NPR3, FAM184B, NID1, PHYHIPL, TMEM121, RASGRF1, SETD1B, TBX4, BNIP3 and/or CCNO.

Other example related to PTEN include one or more of: SGIP1, SYT11, GON4L, STC2, STC2, MTUS2, STK32B, FBXL2, SYK, GBX2, AC079135.1, LMO1, ZNF423, POU4F1, RNF219-AS1, RIN3, HOXD11, HOXB8, HOXB7, AC005597.1, ZNF578, LINC01415, RP11-397A16.1, DPY19L2P1, C10orf54, CDH23, ZCCHC24, C2orf43, OLIG3, BARX1, BARX1-AS1, FAM149A, FAM69B, PCDHA2, PCDHA3, PCDHA1, PCDHA6, PCDHA7, PCDHA4, PCDHA5, CTC-270D5.2, PCDHA9, PCDHAC2, PCDHAC1, PCDHA10, PCDHA11, PCDHA12, PCDHA13, PCDHA8, LRRC32, ST8SIA2, TUB, ROBO3, KIAA0319, SSPN, TBX1, NTF3, FBLL1, RGS17, CTNNA2, KCND2, TNS3, HIF3A, UPP1, ABR, C1QTNF1, C1QTNF1-AS1, FAM19A5, KCTD2, FENDRR, FOXF1, FENDRR, MIR193A, RP1-41C23.4, PLCD1, ZKSCAN7, RP11-62H20.1, LRRC9, EIF1B, EIF1B-AS1, AJ003147.9, ATP10A, MCHR2-AS1, MCHR2, WNT9A, RP11-428C19.4, E2F8, FAM110C, PTEN, KLLN, GSDMD, FES, ADD2, ELMO1, BNIP3, RP11-114H24.4, MIAT, MIAT_exon1, NALCN, RNF219-AS1, RP11-52L5.6, NRN1, CAPN5, OAT, COCH, LPL, GLIS2, GLIS2-AS1, MOB3B, COLCA1, COLCA2, N4BP2L1, RP11-298P3.4, GS1-72M22.1, NEFM, PRMT8, FOXB2, STK25, RP11-2N1.2, CCBE1, ISL1, CTD-2314G24.2, SFRP1, PCDH18, ARMC3, ST6GAL2, WNK2, SLC35C2, RP11-39402.3, LEPR, ADAM19, NIPAL4, CTB-109A12.1, BBC3, MIR3190, TFAP2A, TFAP2A-AS1, RP11-470C13.4, INSM1, and/or LINC01096.

A biomarker is a genetic variant associated with one or more cancers. Biomarkers may be determined using any of several resources or methods. A biomarker may have been previously discovered or may be discovered de novo using experimental or epidemiological techniques. Detection of a biomarker may be indicative of cancer when the biomarker is highly correlated to cancer. Detection of a biomarker may be indicative of cancer when a biomarker in a region or gene occur with a frequency that is greater than a frequency for a given background population or dataset.

In various embodiments, biomarkers, including the aforementioned, may be described as 1) genes encoding transcription factors or signaling factors and 2) the cis regulatory elements that in a positive or negative direction control the expression of those genes. Each of the cis regulatory elements receives multiple inputs from other genes in the network, the inputs being transcription factors which bind to a specific element that contains a specific cis nucleic acid sequence target sites. Functional linkages of which the network is composed are those between the outputs of regulatory genes and the sets of genomic target sites to which their products bind. These functional linkages which orchestrate in both a spatial and temporal fashion the differentiation fate and development plan of a cell or organism establish a network topology that can be analogized to electronic circuitry, its associated switches, capacitors and resistors. A variety of examples are depicted in U.S. Pat. Pub. No. 2014/0234974, which is fully incorporated by reference herein. In this aspect, various transcription factors or signaling factors are nodes in a network topology organized by these functional linkages as organized by the inputs and outputs of regulatory genes.

Publicly available resources such as scientific literature and databases may describe in detail genetic variants found to be associated with cancer. Scientific literature may describe experiments or genome-wide association studies (GWAS) associating one or more genetic variants with cancer. Databases may aggregate information gleaned from sources such as scientific literature to provide a more comprehensive resource for determining one or more biomarkers. Non-limiting examples of databases include FANTOM, GTex, GEO, Body Atlas, INSiGHT, OMIM (Online Mendelian Inheritance in Man, omim.org), cBioPortal (cbioportal.org), CIViC (Clinical Interpretations of Variants in Cancer, civic.genome.wustl.edu), DOCM (Database of Curated Mutations, docm.genome.wustl.edu), and ICGC Data Portal (dcc.icgc.org). In a further example, the COSMIC (Catalogue of Somatic Mutations in Cancer) database allows for searching of biomarkers by cancer, gene, or mutation type. Biomarkers may also be determined de novo by conducting experiments such as case control or association (e.g., genome-wide association studies) studies.

One or more biomarkers may be detected in the sequencing panel. A biomarker may be one or more genetic variants associated with cancer. Biomarkers can be selected from single nucleotide variants (SNVs), copy number variants (CNVs), insertions or deletions (e.g., indels), gene fusions and inversions. Biomarkers may affect the level of a protein. Biomarkers may be in a promoter or enhancer, and may alter the transcription of a gene. The biomarkers may affect the transcription and/or translation efficacy of a gene. The biomarkers may affect the stability of a transcribed mRNA. The biomarker may result in a change to the amino acid sequence of a translated protein. The biomarker may affect splicing, may change the amino acid coded by a particular codon, may result in a frameshift, or may result in a premature stop codon. The biomarker may result in a conservative substitution of an amino acid. One or more biomarkers may result in a conservative substitution of an amino acid. One or more biomarkers may result in a nonconservative substitution of an amino acid.

One or more of the biomarkers may be a driver mutation. A driver mutation is a mutation that gives a selective advantage to a tumor cell in its microenvironment, through either increasing its survival or reproduction. None of the biomarkers may be a driver mutation. One or more of the biomarkers may be a passenger mutation. A passenger mutation is a mutation that has no effect on the fitness of a tumor cell but may be associated with a clonal expansion because it occurs in the same genome with a driver mutation.

The frequency of a biomarker may be as low as 0.001%. The frequency of a biomarker may be as low as 0.005%. The frequency of a biomarker may be as low as 0.01%. The frequency of a biomarker may be as low as 0.02%. The frequency of a biomarker may be as low as 0.03%. The frequency of a biomarker may be as low as 0.05%. The frequency of a biomarker may be as low as 0.1%. The frequency of a biomarker may be as low as 1%.

No single biomarker may be present in more than 50%, of subjects having the cancer. No single biomarker may be present in more than 40%, of subjects having the cancer. No single biomarker may be present in more than 30%, of subjects having the cancer. No single biomarker may be present in more than 20%, of subjects having the cancer. No single biomarker may be present in more than 10%, of subjects having the cancer. No single biomarker may be present in more than 5%, of subjects having the cancer. A single biomarker may be present in 0.001% to 50% of subjects having cancer. A single biomarker may be present in 0.01% to 50% of subjects having cancer. A single biomarker may be present in 0.01% to 30% of subjects having cancer. A single biomarker may be present in 0.01% to 20% of subjects having cancer. A single biomarker may be present in 0.01% to 10% of subjects having cancer. A single biomarker may be present in 0.1% to 10% of subjects having cancer. A single biomarker may be present in 0.1% to 5% of subjects having cancer.

Detection of a biomarker may indicate the presence of one or more cancers. Detection may indicate presence of a cancer selected from the group including ovarian cancer, pancreatic cancer, breast cancer, colorectal cancer, non-small cell lung carcinoma (e.g., squamous cell carcinoma, or adenocarcinoma) or any other cancer. Detection may indicate the presence of any cancer selected from the group including ovarian cancer, pancreatic cancer, breast cancer, colorectal cancer, non-small cell lung carcinoma (squamous cell or adenocarcinoma) or any other cancer. Detection may indicate the presence of any of a plurality of cancers selected from the group including ovarian cancer, pancreatic cancer, breast cancer, colorectal cancer and non-small cell lung carcinoma (squamous cell or adenocarcinoma), or any other cancer. Detection may indicate presence of one or more of any of the cancers mentioned in this application.

One or more cancers may exhibit a biomarker in at least one exon in the panel. One or more cancers selected from the group including ovarian cancer, pancreatic cancer, breast cancer, colorectal cancer, non-small cell lung carcinoma (squamous cell or adenocarcinoma), or any other cancer, each exhibit a biomarker in at least one exon in the panel. Each of at least 3 of the cancers may exhibit a biomarker in at least one exon in the panel. Each of at least 4 of the cancers may exhibit a biomarker in at least one exon in the panel. Each of at least 5 of the cancers may exhibit a biomarker in at least one exon in the panel. Each of at least 8 of the cancers may exhibit a biomarker in at least one exon in the panel. Each of at least 10 of the cancers may exhibit a biomarker in at least one exon in the panel. All of the cancers may exhibit a biomarker in at least one exon in the panel.

If a subject has a cancer, the subject may exhibit a biomarker in at least one exon or gene in the panel. At least 85% of subjects having a cancer may exhibit a biomarker in at least one exon or gene in the panel. At least 90%, of subjects having a cancer may exhibit a biomarker in at least one exon or gene in the panel. At least 92% of subjects having a cancer may exhibit a biomarker in at least one exon or gene in the panel. At least 95% of subjects having a cancer may exhibit a biomarker in at least one exon or gene in the panel. At least 96% of subjects having a cancer may exhibit a biomarker in at least one exon or gene in the panel. At least 97% of subjects having a cancer may exhibit a biomarker in at least one exon or gene in the panel. At least 98% of subjects having a cancer may exhibit a biomarker in at least one exon or gene in the panel. At least 99% of subjects having a cancer may exhibit a biomarker in at least one exon or gene in the panel. At least 99.5% of subjects having a cancer may exhibit a biomarker in at least one exon or gene in the panel.

If a subject has a cancer, the subject may exhibit a biomarker in at least one region in the panel. At least 85% of subjects having a cancer may exhibit a biomarker in at least one region in the panel. At least 90%, of subjects having a cancer may exhibit a biomarker in at least one region in the panel. At least 92% of subjects having a cancer may exhibit a biomarker in at least one region in the panel. At least 95% of subjects having a cancer may exhibit a biomarker in at least one region in the panel. At least 96% of subjects having a cancer may exhibit a biomarker in at least one region in the panel. At least 97% of subjects having a cancer may exhibit a biomarker in at least one region in the panel. At least 98% of subjects having a cancer may exhibit a biomarker in at least one region in the panel. At least 99% of subjects having a cancer may exhibit a biomarker in at least one region in the panel. At least 99.5% of subjects having a cancer may exhibit a biomarker in at least one region in the panel.

Detection may be performed with a high sensitivity and/or a high specificity. Sensitivity can refer to a measure of the proportion of positives that are correctly identified as such. In some cases, sensitivity refers to the percentage of all existing biomarkers that are detected. In some cases, sensitivity refers to the percentage of sick people who are correctly identified as having certain disease. Specificity can refer to a measure of the proportion of negatives that are correctly identified as such. In some cases, specificity refers to the proportion of unaltered bases which are correctly identified. In some cases, specificity refers to the percentage of healthy people who are correctly identified as not having certain disease. The non-unique tagging method described previously significantly increases specificity of detection by reducing noise generated by amplification and sequencing errors, which reduces frequency of false positives. Detection may be performed with a sensitivity of at least 95%, 97%, 98%, 99%, 99.5%, or 99.9% and/or a specificity of at least 80%, 90%, 95%, 97%, 98% or 99%. Detection may be performed with a sensitivity of at least 90%, 95%, 97%, 98%, 99%, 99.5%, 99.6%, 99.98%, 99.9% or 99.95%. Detection may be performed with a specificity of at least 90%, 95%, 97%, 98%, 99%, 99.5%, 99.6%, 99.98%, 99.9% or 99.95%. Detection may be performed with a specificity of at least 70% and a sensitivity of at least 70%, a specificity of at least 75% and a sensitivity of at least 75%, a specificity of at least 80% and a sensitivity of at least 80%, a specificity of at least 85% and a sensitivity of at least 85%, a specificity of at least 90% and a sensitivity of at least 90%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% a sensitivity of 100%. In some cases, the methods can detect a biomarker at a sensitivity of sensitivity of about 80% or greater. In some cases, the methods can detect a biomarker at a sensitivity of sensitivity of about 95% or greater. In some cases, the methods can detect a biomarker at a sensitivity of sensitivity of about 80% or greater, and a sensitivity of sensitivity of about 95% or greater.

Detection may be highly accurate. Accuracy may apply to the identification of biomarkers in cell free DNA, and/or to the diagnosis of cancer. Statistical tools, such as covariate analysis described above, may be used to increase and/or measure accuracy. The methods can detect a biomarker at an accuracy of at least 80%, 90%, 95%, 97%, 98% or 99%, 99.5%, 99.6%, 99.98%, 99.9%, or 99.95%. In some cases, the methods can detect a biomarker at an accuracy of at least 95% or greater.

### Cancer Treatments, Therapies

In some cases, the cancer treatment includes, without limitation, imatinib, gefatinib, afatinib, dacomitinib, sunitinib, sorafenib, vandetanib, brivanib, cabozantib, neratinib, tivantinib, bevacizumab, cixutumumab, dalotuzumab, figitumumab, rilotumumab, onartuzumab, ganitumab, ramucirumab, ridaforolimus, tensirolimus, everolimus, BMS-690514, BMS-754807, EMD 525797, GDC-0973, GDC-0941, MK-2206, AZD6244, GSK1120212, PX-866, XL821, IMC-A12, MM-121, PF-02341066, RG7160, and Sym004. Antibodies suitable for use as anti-EGFR therapy include cetuximab (Trade Name: Erbitux) and panitumumab (Trade Name: Vectibex). In some cases. In some cases, the cancer treatment includes EGFR tyrosine kinase inhibitors such as gefitinib (Trade Name: Iressa), erlotinib (Trade Name: Tarceva), lapatinib, canertinib, and cetuximab.

In some instances, therapies may be used in combination, such as an anti-EGFR therapy and an anti-EGFR therapy. Anti-EGFR therapy may be used in combination with any combination of chemotherapeutic agents or chemotherapeutic regimens, for example, FOLFOX (fluorouracil [5-FU]/leucovorin/oxaliplatin), FOLFIRI (5-FU/leucovorin/irinotecan), and the like.

In some embodiments, the therapy includes an epigenetic regulator, including HAT, HDAC inhibitors as examples. Other examples including, EP015666, LLY-283, JNJ-64619178, BRD0639, AMG193, TNG908, SCR-6920, PRT543, PRT811, MRTX1719, cycloleucine, aminobicycle-hexane-carboxcyclic acid, FIDAS agents, PF-9366, AGR-25696, AG-270, Compound 28, IDE397. Other examples include agents described in Bray et al., Front. Onco. 2023, which is fully incorporated by reference herein.

In some embodiments, the therapy includes paclitaxel (chemotherapeutic drug), ipatasertib (AKT inhibitor), PI3K-Beta inhibitor, AZD8186, docetaxel, tyrosine kinase inhibitor, pazopanib, mTOR inhibitor, everolimus (NCT01430572), PI3K-Beta inhibitor GSK2636771, and immunotherapy, pembrolizumab (NCT03131908), tastuzumab. Other examples include agents described in Ertay et al., Genes and Diseases. 2023, and Dillon and Miller Curr Drug Targets 2015, each of which is fully incorporated by reference herein.

In some aspects, a cancer treatment is administered to a subject. In some cases, the cancer treatment is administered in combination another therapy, such as a non-anti-EGFR therapy with anti-EGFR therapy.

### Sequencing Panel

To improve the likelihood of detecting tumor indicating mutations, the region of DNA sequenced may comprise a panel of genes or genomic regions. Selection of a limited region for sequencing (e.g., a limited panel) can reduce the total sequencing needed (e.g., a total amount of nucleotides sequenced. A sequencing panel can target a plurality of different genes or regions to detect a single cancer, a set of cancers, or all cancers.

In some aspects, a panel targets a plurality of different genes or genomic regions is selected such that a determined proportion of subjects having a cancer exhibits a genetic variant or biomarker in one or more different genes or genomic regions in the panel. The panel may be selected to limit a region for sequencing to a fixed number of base pairs. The panel may be selected to sequence a desired amount of DNA. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity and/or a theoretical accuracy for detecting one or more genetic variants in a sample.

Probes for detecting the panel of regions can include those for detecting hotspots regions as well as nucleosome-aware probes (e.g., KRAS codons 12 and 13) and may be designed to optimize capture based on analysis of cfDNA coverage and fragment size variation impacted by nucleosome binding patterns and GC sequence composition. Regions used herein can also include non-hotspot regions optimized based on nucleosome positions and GC models. The panel can comprise a plurality of subpanels, including subpanels for identifying tissue of origin (e.g., use of published literature to define 50-100 baits representing genes with most diverse transcription profile across tissues (not necessarily promoters)), whole genome scaffold (e.g., for identifying ultra-conservative genomic content and tiling sparsely across chromosomes with handful of probes for copy number base lining purposes), transcription start site (TSS)/CpG islands (e.g., for capturing differential methylated regions (e.g., Differentially Methylated Regions (DMRs)) in for example in promoters of tumor suppressor genes (e.g., SEPT9/VIM in colorectal cancer)). In some embodiments, markers for a tissue of origin are tissue-specific epigenetic markers.

The one or more regions in the panel can comprise one or more loci from one or a plurality of genes. The plurality of genes may be selected for sequencing and biomarker detection. Genes included in the region to be sequenced may be selected from genes known to be involved in cancer, or from genes not involved in cancer. For example the plurality of genes in the panel may be oncogenes, tumor suppressors, growth factors, DNA repair genes, signaling genes, transcription factors, receptors or metabolic genes.

Example related to MTAP include one or more of: CDKN2A-DT, ELAVL2, THBD, CYP27C1, SH3BP2, TXNRD1, ESYT3, LOC105378933, MN1, HTRA3, IDH2, IDH2-DT, ELFN2, ACTC1, PTGS2, PACERR, WWC2, WWC2-AS2, OSMR-AS1, OSMR, CHST15, COL18A1, TGFBI, SHISA2, HACD4, PRKG1-AS1, DKK1, CCK, CLIP4, HIF3A, GPR156, MIR6068, CDKN2B-AS1, CDKN2A, VLDLR-AS1, PTHLH, H2BP2, PAK5, COL4A3, COL4A4, BMPR1B-DT, BMPR1B, PCDHGC3, PLA2R1, RBPMS-AS1, CAV2, RASL12, PPFIA2, TIAM1, PAPLN, MIR31HG, LOXL2, LRRC71, MAMDC2, CSGALNACT1, GLP1R, PDGFD, EPB41L4A, EPB41L4A-DT, TMEM200B, CYP19A1, GLDN, SLC9A7P1, NXNL2, LOC100130207, IER3, FLOT1, IER3-AS1, C7orf57, GPR12, CXCL1, SPAG17, BEGAIN, HSD52, UHRF2, ERC2, NT5E, MAP10, MIR4785, LINC01574, DMRTA1, AKR1B1, SPOCK3, CASR, ALKALI, FMN1, CHST11, STK32A, STK32A-AS1, LOC100507144, CD44, AFAP1L2, ICAM1, RAD52, WIPF1, EMBP1, SLC6A20, RBP7, CHRNB4, KCNS2, DOCK8, GALNT16, CPNE8, DUOXA1, DUOX1, FRMD5, FAM217B, SYCP2, FLNC, SCHIP1, RPL39L, LINC02609, LINC01763, and/or CD40.

Additional examples related to MTAP include one or more of TRIM67, PCDH8, OLIG2, AP000282.2, RP11-65J21.3, MIR193B, RP11-65J21.3, LTBP2, NKX2-1-AS1, GLYATL2, GLYATL1, RP11-522I20.3, UBQLN1, RP11-147L13.2, AMZ2, SQSTM1, MGAT4B, PRR14, ADAMTS20, FOXG1-AS1, FOXG1, TCTEX1D1, EVL, TOR1B, BARHL2, OSTM1, NR2E1, ACSF2, MIR193A, RP1-41C23.4, CCDC178, ABCC9, CACNG8, ANKRD20A8P, NRN1, DNMT3A, ITGA8, RP11-501C14.5, IGF2BP1, RP11-501C14.5, ELMO1, MICU3, FOXF2, ZNF775, TGFBR1, RAG1, TRAF6, RP11-545D22.1, ZNF607, CTD-2528L19.4, RP11-742D12.2, ST8SIA5, OTX2, ATG12, AP3S1, AJ003147.9, RP11-816J6.3, ACTRT3, TET2, BVES, BVES-AS1, NT5E, PCDHGA10, PCDHGB1, PCDHGB3, PCDHGB2, PCDHGA8, PCDHGA9, PCDHGB7, PCDHGB6, PCDHGB5, PCDHGB4, PCDHGA2, PCDHGA3, PCDHGA1, PCDHGA6, PCDHGA7, PCDHGA4, PCDHGA5, CNPY1, AC008060.5, CCDC140, PAX3, ZNF667-AS1, ZNF667, TMEM155, PP12613, CTC-512J12.4, CTC-512J12.6, ZNF285, EPB41L3, GSX1, CDH3, SMC5-AS1, SMC5, ELAVL4, PCOLCE2, CCDC140, PAX3, SPAG6, PDCL, HNF1B, ZNF790-AS1, SORCS3, PNMA1, PRMT3, FAM183A, DLX2-AS1, DLX2, EPB41L3, SOX11, LINC01248, AC108025.2, SOX11, LINC01248, SOX11, HOXA11-AS, RP1-170019.14, ALDH7A1, GPR158-AS1, GPR158, RAI1, AC093887.1, POU4F2, GALT, RP11-195F19.29, AC008753.6, MYADM, ST20-AS1, ST20, ST20-MTHFS, RP11-81A1.6, ST20, ST20-MTHFS, ST20-AS1, KLHL9, RP11-744N12.3, FLI1, RYR1, KCNIP2, SLC6A2, LAMA1, GSG1L, CUX2, RXRG, SH3GL3, KIRREL, NCR3LG1, AC093627.12, and/or FAM20C.

Other examples related to PTEN include one or more of: HOXC-AS3, FGF8, TJP1, RHBDD1, IRS1, ATP10A, HTR1B, BASP1, CHMP4A, MEIS1, CH25H, SYT5, HOXB5, HOXC-AS3, CDHR5, SCT, MIR6719, DYRKIB, PAH, IL12RB2, PMP22, H2BC3, FOXCUT, FOXC1, CXXC5, PAM, PCDHAC1, GATA4, OSR1, MIR193B, MIR193BHG, RUSC1, PIK3R3, SESN3, CACNAlC-AS1, HOXB-AS3, GHRL, LINC02560, STK3, OSR2, ZNF565, RHBDD1, BHLHE40, BHLHE40-AS1, CAST, ACTC1, OSBP2, RECQL4, HMGA1, MIR6835, EYA1, LOC101929340, SHISA8, H3C8, H2BC10, LINC01006, PRDM16, PRDM16-DT, LINC01833, ANKRD45, LOC105379152, ADHFE1, RRS1-AS1, H3C7, H4C7, E2F8, TBX1, TBX15, HPN, CALHM2, NPPB, LBX2, DERL3, NKX3-2, HOXB5, PHPT1, KCNJ3, HSF4, TRADD, RPP25, SIM2, STX16-NPEPL1, STX16, PTTG1IP, ZNF292, SPEG, FES, PKP1, LHX1, HOXB3, PRDM16-DT, MMP23B, AGMAT, DUOX2, NKX2-2, TBX6, LOC101928595, MVP, PAGR1, HEBP1, CRYM, NTRK2, LOC145845, ATP13A3, TJP1, SERHL, ARHGEF19, LOC100507634, MIR4422HG, USP24, RASSF1, RASSF1-AS1, RHOH, BARX2, FILIPIL, CMSS1, ZNF546, RARA-AS1, USP3, CBX4, SPATA18, ODAD2P1, SETBP1, NUTF2, THAP11, C6orf141, RND2, VAT1, and/or CAMTA2.

Additional examples related to PTEN include one or more of: SGIP1, SYT11, GON4L, STC2, STC2, MTUS2, STK32B, FBXL2, SYK, GBX2, AC079135.1, LMO1, ZNF423, POU4F1, RNF219-AS1, RIN3, HOXD11, HOXB8, HOXB7, AC005597.1, ZNF578, LINC01415, RP11-397A16.1, DPY19L2P1, C10orf54, CDH23, ZCCHC24, C2orf43, OLIG3, BARX1, BARX1-AS1, FAM149A, FAM69B, PCDHA2, PCDHA3, PCDHA1, PCDHA6, PCDHA7, PCDHA4, PCDHA5, CTC-270D5.2, PCDHA9, PCDHAC2, PCDHAC1, PCDHA10, PCDHA11, PCDHA12, PCDHA13, PCDHA8, LRRC32, ST8SIA2, TUB, ROBO3, KIAA0319, SSPN, TBX1, NTF3, FBLL1, RGS17, CTNNA2, KCND2, TNS3, HIF3A, UPP1, ABR, C1QTNF1, C1QTNF1-AS1, FAM19A5, KCTD2, FENDRR, FOXF1, FENDRR, MIR193A, RP1-41C23.4, PLCD1, ZKSCAN7, RP11-62H20.1, LRRC9, EIF1B, EIF1B-AS1, AJ003147.9, ATP10A, MCHR2-AS1, MCHR2, WNT9A, RP11-428C19.4, E2F8, FAM110C, PTEN, KLLN, GSDMD, FES, ADD2, ELMO1, BNIP3, RP11-114H24.4, MIAT, MIAT_exon1, NALCN, RNF219-AS1, RP11-52L5.6, NRN1, CAPN5, OAT, COCH, LPL, GLIS2, GLIS2-AS1, MOB3B, COLCA1, COLCA2, N4BP2L1, RP11-298P3.4, GS1-72M22.1, NEFM, PRMT8, FOXB2, STK25, RP11-2N1.2, CCBE1, ISL1, CTD-2314G24.2, SFRP1, PCDH18, ARMC3, ST6GAL2, WNK2, SLC35C2, RP11-39402.3, LEPR, ADAM19, NIPAL4, CTB-109A12.1, BBC3, MIR3190, TFAP2A, TFAP2A-AS1, RP11-470C13.4, INSM1, and/or LINC01096.

Other examples related to EGFR include one or more of: EGFR, CPZ, GPR78, TOX2, ADCY8, GAS7, TRIM71, SRRM4, RP4-563E14.1, DIDO1, VPS37B, P2RX2, NIM1K, ZNF131, NIM1K, OPRK1, CLDN10, STMN3, LINC00472, VIM-AS1, VIM, RP11-32K4.1, PVR, CTB-171A8.1, CACNA1G-AS1, CACNA1G, CCND2-AS1, CCND2-AS2, CCND2, CCND2-AS1, CCND2-AS2, RHEB, MIR6068, ANKRD63, PLCB2, FOXJ1, RP11-449J21.5, LHX5, GRID1, PAX7, LINC00925, PIK3R5, CTB-41I6.1, LIMS2, ZNF132, CTD-2619J13.19, PTPRO, RERG, WIF1, FAIM2, C5orf38, IRX2, SOX1, HSP90AA1, NACAD, ANK1, GPR158-AS1, GPR158, BEND6, DST, EFCC1, KIAA1257, SUN1, C9orf50, NTMT1, EYA2, ZSCAN31, KAZN, ADAMTS5, ZFP37, SETD1B, SETD1B, LINC01089, RHOF, SETD1B, LINC01089, RP11-467K18.2, NRIP3, ZFPM2, MIR193A, RP1-41C23.4, SHISA6, GLT1D1, RP11-672L10.2, RP11-672L10.3, ADCYAP1, RP11-379F12.4, GATA3-AS1, RP11-379F12.4, FGF14, RIMS4, RNF220, FES, ARRDC2, SEZ6L, USH1C, RAMP2-AS1, RAMP2, WNT2, ZIK1, TOX2, AAAS, ADAMTS18, RP11-449J10.1, RP11-713D19.1, SPDYA, NPR3, FAM184B, NID1, PHYHIPL, TMEM121, RASGRF1, SETD1B, TBX4, BNIP3 and/or CCNO.

Other example related to PTEN include one or more of: SGIP1, SYT11, GON4L, STC2, STC2, MTUS2, STK32B, FBXL2, SYK, GBX2, AC079135.1, LMO1, ZNF423, POU4F1, RNF219-AS1, RIN3, HOXD11, HOXB8, HOXB7, AC005597.1, ZNF578, LINC01415, RP11-397A16.1, DPY19L2P1, C10orf54, CDH23, ZCCHC24, C2orf43, OLIG3, BARX1, BARX1-AS1, FAM149A, FAM69B, PCDHA2, PCDHA3, PCDHA1, PCDHA6, PCDHA7, PCDHA4, PCDHA5, CTC-270D5.2, PCDHA9, PCDHAC2, PCDHAC1, PCDHA10, PCDHA11, PCDHA12, PCDHA13, PCDHA8, LRRC32, ST8SIA2, TUB, ROBO3, KIAA0319, SSPN, TBX1, NTF3, FBLL1, RGS17, CTNNA2, KCND2, TNS3, HIF3A, UPP1, ABR, C1QTNF1, C1QTNF1-AS1, FAM19A5, KCTD2, FENDRR, FOXF1, FENDRR, MIR193A, RP1-41C23.4, PLCD1, ZKSCAN7, RP11-62H20.1, LRRC9, EIF1B, EIF1B-AS1, AJ003147.9, ATP10A, MCHR2-AS1, MCHR2, WNT9A, RP11-428C19.4, E2F8, FAM110C, PTEN, KLLN, GSDMD, FES, ADD2, ELMO1, BNIP3, RP11-114H24.4, MIAT, MIAT_exon1, NALCN, RNF219-AS1, RP11-52L5.6, NRN1, CAPN5, OAT, COCH, LPL, GLIS2, GLIS2-AS1, MOB3B, COLCA1, COLCA2, N4BP2L1, RP11-298P3.4, GS1-72M22.1, NEFM, PRMT8, FOXB2, STK25, RP11-2N1.2, CCBE1, ISL1, CTD-2314G24.2, SFRP1, PCDH18, ARMC3, ST6GAL2, WNK2, SLC35C2, RP11-39402.3, LEPR, ADAM19, NIPAL4, CTB-109A12.1, BBC3, MIR3190, TFAP2A, TFAP2A-AS1, RP11-470C13.4, INSM1, and/or LINC01096.

Examples of genes that may be in the panel include, but are not limited to: APC, AR, ARID1A, BRAF, BRCA1, BRCA2, CCND1, CCND2, CCNE1, CDK4, CDK6, CDKN2A, CDKN2B, EGFR, ERBB2, FGFR1, FGFR2, HRAS, KIT, KRAS, MET, MYC, NF1, NRAS, PDGFRA, PIK3CA, PTEN, RAF1, TP53, AKT1, ALK, ARAF, ATM, CDH1, CTNNB1, ESR1, EZH2, FBXW7, FGFR3, GATA3, GNA11, GNAQ, GNAS, HNF1A, IDH1, IDH2, JAK2, JAK3, MAP2K1, MAP2K2, MLH1, MPL, NFE2L2, NOTCH1, NPM1, NTRK1, PTPN11, RET, RHEB, RHOA, RIT1, ROS1, SMAD4, SMO, SRC, STK11, TERT, VHL.

In some cases, the one or more regions in the panel can comprise one or more loci from one or a plurality of genes, including one or more of AKT1, ALK, APC, ATM, BRAF, CTNNB1, EGFR, ERBB2, ESR1, FGFR2, GATA3, GNAS, IDH1, IDH2, KIT, KRAS, MET, NRAS, PDGFRA, PIK3CA, PTEN, RB1, SMAD4, STK11, and TP53.

In some cases, the one or more regions in a panel for colorectal cancer can comprise one or more loci from one or a plurality of genes, including one of, two of, three of, four of, or five of TP53, APC, BRAF, KRAS, and NRAS. In some cases, the one or more regions in a panel for ovarian cancer can comprise one or more loci from one or a plurality of genes, including TP53. In some cases, the one or more regions in a panel for pancreatic cancer can comprise one or more loci from one or a plurality of genes, including one or both of TP53 and KRAS. In some cases, the one or more regions in a panel for lung adenocarcinoma can comprise one or more loci from one or a plurality of genes, including one of, two of, three of, four of, five of, six of, seven of, or eight of TP53, BRAF, KRAS, EGFR, ERBB2, MET, STK11, and ALK. In some cases, the one or more regions in a panel for lung squamous cell carcinoma can comprise one or more loci from one or a plurality of genes, including one of, two of, three of, four of, or five of TP53, BRAF, KRAS, MET, and ALK. In some cases, the one or more regions in a panel for breast cancer can comprise one or more loci from one or a plurality of genes, including one of, two of, three of, or four of TP53, GATA3, PIK3CA, and ESR1. In some cases, one or more regions in a panel can comprise one or more loci from a combination of any of the above genes, for example, to detect a combination of cancer types. In some cases, one or more regions in a panel can comprise one or more loci from each of the preceding genes, for example, in a pan-cancer panel.

In some cases, the one or more regions in a panel for lung cancer can comprise one or more loci from a plurality of genes, including one of, two of, three of, four of, five of, six of, seven of, eight of, nine of, 10 of, 11 of, 12 of, 13 of, 14 of, 15 of, 16 of, 17 of, 18 of, 19 of, or 20 of EGFR, KRAS, TP53, CDKN2A, STK11, BRAF, PIK3CA, RB1, ERBB2, PTEN, NFE2L2, MET, CTNNB1, NRAS, MUC16, NF1, BAI3, SMARCA4, ATM, NTRK3, and ERBB4. Such a panel also may include, or have substituted for any or all of the above, any or all of an EGFR Exon 19 deletion, EGFR L858R, EGFR C797S, EGFR T790M, EGFR S645C, ARAF S214C and S214F, ERBB2 S418T, MET exon 14 skipping, SNVs and indels. Many of these genes may be clinically actionable, such that an observed anomaly in MAF (e.g., significantly higher or lower than in normal control subjects) may be indicative of a clinical state relevant to lung cancer, such as diagnosis, prognosis, risk stratification, treatment selection, tumor resistance to treatment, tumor burden, etc. Such a lung cancer targeted panel may comprise a relatively small number of these lung cancer associated genes.

In some cases, the one or more regions in a panel for breast cancer can comprise one or more loci from a plurality of genes, including any one of, or any combination of, ACVRL1, AFF2, AGMO, AGTR2, AHNAK, AHNAK2, AKAP9, AKT1, AKT2, ALK, APC, ARID1A, ARIDIB, ARID2, ARID5B, ASXL1, ASXL2, ATR, BAP1, BCAS3, BIRC6, BRAF, BRCA1, BRCA2, BRIP1, CACNA2D3, CASP8, CBFB, CCND3, CDH1, CDKN1B, CDKN2A, CHD1, CHEK2, CLK3, CLRN2, COL12A1, COL22A1, COL6A3, CTCF, CTNNA1, CTNNA3, DCAF4L2, DNAH11, DNAH2, DNAH5, DTWD2, EGFR, EP300, ERBB2, ERBB3, ERBB4, FAM20C, FANCA, FANCD2, FBXW7, FLT3, FOXO1, FOXO3, FOXP1, FRMD3, GATA3, GH1, GLDC, GPR124, GPR32, GPS2, HDAC9, HERC2, HIST1H2BC, HRAS, JAK1, KDM3A, KDM6A, KLRG1, KMT2C, KRAS, L1CAM, LAMA2, LAMB3, LARGE, LDLRAP1, LIFR, LIPI, MAGEA8, MAP2K4, MAP3K1, MAP3K10, MAP3K13, MBL2, MEN1, LL2, MLLT4, MTAP, MUC16, MYH9, MYO1A, MYO3A, NCOA3, NCOR1, NCOR2, NDFIP1, NEK1, NF1, NF2, NOTCHI, NPNT, NR2F1, NR3C1, NRAS, NRG3, NT5E, OR6A2, PALLD, PBRM1, PDE4DIP, PIK3CA, PIK3R1, PPP2CB, PPP2R2A, PRKACG, PRKCE, PRKCQ, PRKCZ, PRKG1, PRPS2, PRR16, PTEN, PTPN22, PTPRD, PTPRM, RASGEFIB, RB1, ROS1, RPGR, RUNX1, RYR2, SBNO1, SETD1A, SETD2, SETDB1, SF3B1, SGCD, SHANK2, SIAH1, SIK1, SIK2, SMAD2, SMAD4, SMARCB1, SMARCC1, SMARCC2, SMARCD1, SPACA1, STAB2, STK11, STMN2, SYNE1, TAF1, TAF4B, TBL1XR1, TBX3, TG, THADA, THSD7A, TP53, TTYH1, UBR5, USH2A, USP28, USP9X, UTRN, and ZFP36L1. Many of these genes may be clinically actionable, such that an observed anomaly in MAF (e.g., significantly higher or lower than in normal control subjects) may be indicative of a clinical state relevant to breast cancer, such as diagnosis, prognosis, risk stratification, treatment selection, tumor resistance to treatment, tumor burden, etc. Such a breast cancer targeted panel may comprise a relatively small number of these breast cancer associated genes.

In some cases, the one or more regions in a panel for colorectal cancer can comprise one or more loci from a plurality of genes, including one of, two of, three of, four of, five of, or six of TP53, BRAF, KRAS, APC, TGFBR, and PIK3CA. Many of these genes may be clinically actionable, such that an observed anomaly in MAF (e.g., significantly higher or lower than in normal control subjects) may be indicative of a clinical state relevant to colorectal cancer, such as diagnosis, prognosis, risk stratification, treatment selection, tumor resistance to treatment, tumor burden, etc. Such a colorectal cancer targeted panel may comprise a relatively small number of these colorectal cancer associated genes.

In some embodiments, the one or more regions in the panel comprise one or more loci from one or a plurality of genes for detecting residual cancer after surgery. This detection can be earlier than is possible for existing methods of cancer detection. In some embodiments, the one or more regions in the panel comprise one or more loci from one or a plurality of genes for detecting cancer in a high-risk patient population. For example, smokers have much higher rates of lung cancer than the general population. Moreover, smokers can develop other lung conditions that make cancer detection more difficult, such as the development of irregular nodules in the lungs. In some embodiments, the methods described herein detect cancer in high risk patients earlier than is possible for existing methods of cancer detection.

A region may be selected for inclusion in a sequencing panel based on a number of subjects with a cancer that have a biomarker in that gene or region. A region may be selected for inclusion in a sequencing panel based on prevalence of subjects with a cancer and a biomarker present in that gene. Presence of a biomarker in a region may be indicative of a subject having cancer.

In some instances, the panel may be selected using information from one or more databases. The information regarding a cancer may be derived from cancer tumor biopsies or cfDNA assays. A database may comprise information describing a population of sequenced tumor samples. A database may comprise information about mRNA expression in tumor samples. A databased may comprise information about regulatory elements in tumor samples. The information relating to the sequenced tumor samples may include the frequency various genetic variants and describe the genes or regions in which the genetic variants occur. The genetic variants may be biomarkers. A non-limiting example of such a database is COSMIC. COSMIC is a catalogue of somatic mutations found in various cancers. For a particular cancer, COSMIC ranks genes based on frequency of mutation. A gene may be selected for inclusion in a panel by having a high frequency of mutation within a given gene. For instance, COSMIC indicates that 33% of a population of sequenced breast cancer samples have a mutation in TP53 and 22% of a population of sampled breast cancers have a mutation in KRAS. Other ranked genes, including APC, have mutations found only in about 4% of a population of sequenced breast cancer samples. TP53 and KRAS may be included in a sequencing panel based on having relatively high frequency among sampled breast cancers (compared to APC, for example, which occurs at a frequency of about 4%). COSMIC is provided as a non-limiting example, however, any database or set of information may be used that associates a cancer with biomarker located in a gene or genetic region. In another example, as provided by COSMIC, of 1156 biliary tract cancer samples, 380 samples (33%) carried mutations in TP53. Several other genes, such as APC, have mutations in 4-8% of all samples. Thus, TP53 may be selected for inclusion in the panel based on a relatively high frequency in a population of biliary tract cancer samples.

A gene or region may be selected for a panel where the frequency of a biomarker is significantly greater in sampled tumor tissue or circulating tumor DNA than found in a given background population. A combination of regions may be selected for inclusion of a panel such that at least a majority of subjects having a cancer will have a biomarker present in at least one of the regions or genes in the panel. The combination of regions may be selected based on data indicating that, for a particular cancer or set of cancers, a majority of subjects have one or more biomarkers in one or more of the selected regions. For example, to detect cancer 1, a panel including regions A, B, C, and/or D may be selected based on data indicating that 90% of subjects with cancer 1 have a biomarker in regions A, B, C, and/or D of the panel. Alternately, biomarkers may be shown to occur independently in two or more regions in subjects having a cancer such that, combined, a biomarker in the two or more regions is present in a majority of a population of subjects having a cancer. For example, to detect cancer 2, a panel including regions X, Y, and Z may be selected based on data indicating that 90% of subjects have a biomarker in one or more regions, and in 30% of such subjects a biomarker is detected only in region X, while biomarkers are detected only in regions Y and/or Z for the remainder of the subjects for whom a biomarker was detected. Biomarkers present in one or more regions previously shown to be associated with one or more cancers may be indicative of or predictive of a subject having cancer if a biomarker is detected in one or more of those regions 50% or more of the time. Computational approaches such as models employing conditional probabilities of detecting cancer given a known cancer frequency for a set of biomarkers within one or more regions may be used to predict which regions, alone or in combination, may be predictive of cancer. Other approaches for panel selection involve the use of databases describing information from studies employing comprehensive genomic profiling of tumors with large panels and/or whole genome sequencing (WGS, RNA-seq, Chip-seq, bisulfate sequencing, ATAC-seq, and others). Information gleaned from literature may also describe pathways commonly affected and mutated in certain cancers. Panel selection may be further informed by the use of ontologies describing genetic information.

Genes included in the panel for sequencing can include the fully transcribed region, the promoter region, enhancer regions, regulatory elements, and/or downstream sequence. To further increase the likelihood of detecting tumor indicating mutations only exons may be included in the panel. The panel can comprise all exons of a selected gene, or only one or more of the exons of a selected gene. The panel may comprise of exons from each of a plurality of different genes. The panel may comprise at least one exon from each of the plurality of different genes.

In some aspects, a panel of exons from each of a plurality of different genes is selected such that a determined proportion of subjects having a cancer exhibit a genetic variant in at least one exon in the panel of exons.

At least one full exon from each different gene in a panel of genes may be sequenced. The sequenced panel may comprise exons from a plurality of genes. The panel may comprise exons from 2 to 100 different genes, from 2 to 70 genes, from 2 to 50 genes, from 2 to 30 genes, from 2 to 15 genes, or from 2 to 10 genes.

A selected panel may comprise a varying number of exons. The panel may comprise from 2 to 3000 exons. The panel may comprise from 2 to 1000 exons. The panel may comprise from 2 to 500 exons. The panel may comprise from 2 to 100 exons. The panel may comprise from 2 to 50 exons. The panel may comprise no more than 300 exons. The panel may comprise no more than 200 exons. The panel may comprise no more than 100 exons. The panel may comprise no more than 50 exons. The panel may comprise no more than 40 exons. The panel may comprise no more than 30 exons. The panel may comprise no more than 25 exons. The panel may comprise no more than 20 exons. The panel may comprise no more than 15 exons. The panel may comprise no more than 10 exons. The panel may comprise no more than 9 exons. The panel may comprise no more than 8 exons. The panel may comprise no more than 7 exons.

The panel may comprise one or more exons from a plurality of different genes. The panel may comprise one or more exons from each of a proportion of the plurality of different genes. The panel may comprise at least two exons from each of at least 25%, 50%, 75% or 90% of the different genes. The panel may comprise at least three exons from each of at least 25%, 50%, 75% or 90% of the different genes. The panel may comprise at least four exons from each of at least 25%, 50%, 75% or 90% of the different genes.

The sizes of the sequencing panel may vary. A sequencing panel may be made larger or smaller (in terms of nucleotide size) depending on several factors including, for example, the total amount of nucleotides sequenced or a number of unique molecules sequenced for a particular region in the panel. The sequencing panel can be sized 5 kb to 50 kb. The sequencing panel can be 10 kb to 30 kb in size. The sequencing panel can be 12 kb to 20 kb in size. The sequencing panel can be 12 kb to 60 kb in size. The sequencing panel can be at least 10 kb, 12 kb, 15 kb, 20 kb, 25 kb, 30 kb, 35 kb, 40 kb, 45 kb, 50 kb, 60 kb, 70 kb, 80 kb, 90 kb, 100 kb, 110 kb, 120 kb, 130 kb, 140 kb, or 150 kb in size. The sequencing panel may be less than 100 kb, 90 kb, 80 kb, 70 kb, 60 kb, or 50 kb in size.

The panel selected for sequencing can comprise at least 1, 5, 10, 15, 20, 25, 30, 40, 50, 60, 80, or 100 regions. In some cases, the regions in the panel are selected that the size of the regions are relatively small. In some cases, the regions in the panel have a size of about 10 kb or less, about 8 kb or less, about 6 kb or less, about 5 kb or less, about 4 kb or less, about 3 kb or less, about 2.5 kb or less, about 2 kb or less, about 1.5 kb or less, or about 1 kb or less or less. In some cases, the regions in the panel have a size from about 0.5 kb to about 10 kb, from about 0.5 kb to about 6 kb, from about 1 kb to about 11 kb, from about 1 kb to about 15 kb, from about 1 kb to about 20 kb, from about 0.1 kb to about 10 kb, or from about 0.2 kb to about 1 kb. For example, the regions in the panel can have a size from about 0.1 kb to about 5 kb.

The panel selected herein can allow for deep sequencing that is sufficient to detect low-frequency genetic variants (e.g., in cell-free nucleic acid molecules obtained from a sample). An amount of genetic variants in a sample may be referred to in terms of the minor allele frequency for a given genetic variant. The minor allele frequency may refer to the frequency at which minor alleles (e.g., not the most common allele) occurs in a given population of nucleic acids, such as a sample. Genetic variants at a low minor allele frequency may have a relatively low frequency of presence in a sample. In some cases, the panel allows for detection of genetic variants at a minor allele frequency of at least 0.0001%, 0.001%, 0.005%, 0.01%, 0.05%, 0.1%, or 0.5%. The panel can allow for detection of genetic variants at a minor allele frequency of 0.001% or greater. The panel can allow for detection of genetic variants at a minor allele frequency of 0.01% or greater. The panel can allow for detection of genetic variant present in a sample at a frequency of as low as 0.0001%, 0.001%, 0.005%, 0.01%, 0.025%, 0.05%, 0.075%, 0.1%, 0.25%, 0.5%, 0.75%, or 1.0%. The panel can allow for detection of biomarkers present in a sample at a frequency of at least 0.0001%, 0.001%, 0.005%, 0.01%, 0.025%, 0.05%, 0.075%, 0.1%, 0.25%, 0.5%, 0.75%, or 1.0%. The panel can allow for detection of biomarkers at a frequency in a sample as low as 1.0%. The panel can allow for detection of biomarkers at a frequency in a sample as low as 0.75%. The panel can allow for detection of biomarkers at a frequency in a sample as low as 0.5%. The panel can allow for detection of biomarkers at a frequency in a sample as low as 0.25%. The panel can allow for detection of biomarkers at a frequency in a sample as low as 0.1%. The panel can allow for detection of biomarkers at a frequency in a sample as low as 0.075%. The panel can allow for detection of biomarkers at a frequency in a sample as low as 0.05%. The panel can allow for detection of biomarkers at a frequency in a sample as low as 0.025%. The panel can allow for detection of biomarkers at a frequency in a sample as low as 0.01%. The panel can allow for detection of biomarkers at a frequency in a sample as low as 0.005%. The panel can allow for detection of biomarkers at a frequency in a sample as low as 0.001%. The panel can allow for detection of biomarkers at a frequency in a sample as low as 0.0001%. The panel can allow for detection of biomarkers in sequenced cfDNA at a frequency in a sample as low as 1.0% to 0.0001%. The panel can allow for detection of biomarkers in sequenced cfDNA at a frequency in a sample as low as 0.01% to 0.0001%.

A genetic variant can be exhibited in a percentage of a population of subjects who have a disease (e.g., cancer). In some cases, at least 1%, 2%, 3%, 5%, 10%, 20%, 30%, 40%, 50%, 60%, 70%, 80%, 90%, 95%, or 99% of a population having the cancer exhibit one or more genetic variants in at least one of the regions in the panel. For example, at least 80% of a population having the cancer may exhibit one or more genetic variants in at least one of the regions in the panel.

The panel can comprise one or more regions from each of one or more genes. In some cases, the panel can comprise one or more regions from each of at least 1, 2, 3, 4, 5, 6, 7, 8, 9, 10, 15, 20, 25, 30, 40, 50, or 80 genes. In some cases, the panel can comprise one or more regions from each of at most 1, 2, 3, 4, 5, 6, 7, 8, 9, 10, 15, 20, 25, 30, 40, 50, or 80 genes. In some cases, the panel can comprise one or more regions from each of from about 1 to about 80, from 1 to about 50, from about 3 to about 40, from 5 to about 30, from 10 to about 20 different genes.

The regions in the panel can be selected so that one or more epigenetically modified regions are detected. The one or more epigenetically modified regions can be acetylated, methylated, ubiquitylated, phosphorylated, sumoylated, ribosylated, and/or citrullinated. For example, the regions in the panel can be selected so that one or more methylated regions are detected.

The regions in the panel can be selected so that they comprise sequences differentially transcribed across one or more tissues. In some cases, the regions can comprise sequences transcribed in certain tissues at a higher level compared to other tissues. For example, the regions can comprise sequences transcribed in certain tissues but not in other tissues.

The regions in the panel can comprise coding and/or non-coding sequences. For example, the regions in the panel can comprise one or more sequences in exons, introns, promoters, 3′ untranslated regions, 5′ untranslated regions, regulatory elements, transcription start sites, and/or splice sites. In some cases, the regions in the panel can comprise other non-coding sequences, including pseudogenes, repeat sequences, transposons, viral elements, and telomeres. In some cases, the regions in the panel can comprise sequences in non-coding RNA, e.g., ribosomal RNA, transfer RNA, Piwi-interacting RNA, and microRNA.

The regions in the panel can be selected to detect (diagnose) a cancer with a desired level of sensitivity (e.g., through the detection of one or more genetic variants). For example, the regions in the panel can be selected to detect the cancer (e.g., through the detection of one or more genetic variants) with a sensitivity of at least 50%, 55%, 60%, 65%, 70%, 75%, 80%, 85%, 90%, 95%, 96%, 97%, 98%, 99%, 99.5%, or 99.9%. The regions in the panel can be selected to detect the cancer with a sensitivity of 100%.

The regions in the panel can be selected to detect (diagnose) a cancer with a desired level of specificity (e.g., through the detection of one or more genetic variants). For example, the regions in the panel can be selected to detect cancer (e.g., through the detection of one or more genetic variants) with a specificity of at least 50%, 55%, 60%, 65%, 70%, 75%, 80%, 85%, 90%, 95%, 96%, 97%, 98%, 99%, 99.5%, or 99.9%. The regions in the panel can be selected to detect the one or more genetic variant with a specificity of 100%.

The regions in the panel can be selected to detect (diagnose) a cancer with a desired positive predictive value. Positive predictive value can be increased by increasing sensitivity (e.g., chance of an actual positive being detected) and/or specificity (e.g., chance of not mistaking an actual negative for a positive). As a non-limiting example, regions in the panel can be selected to detect the one or more genetic variant with a positive predictive value of at least 50%, 55%, 60%, 65%, 70%, 75%, 80%, 85%, 90%, 95%, 96%, 97%, 98%, 99%, 99.5%, or 99.9%. The regions in the panel can be selected to detect the one or more genetic variant with a positive predictive value of 100%.

The regions in the panel can be selected to detect (diagnose) a cancer with a desired accuracy. As used herein, the term “accuracy” may refer to the ability of a test to discriminate between a disease condition (e.g., cancer) and health. Accuracy may be can be quantified using measures such as sensitivity and specificity, predictive values, likelihood ratios, the area under the ROC curve, Youden's index and/or diagnostic odds ratio.

Accuracy may presented as a percentage, which refers to a ratio between the number of tests giving a correct result and the total number of tests performed. The regions in the panel can be selected to detect cancer with an accuracy of at least 50%, 55%, 60%, 65%, 70%, 75%, 80%, 85%, 90%, 95%, 96%, 97%, 98%, 99%, 99.5%, or 99.9%. The regions in the panel can be selected to detect cancer with an accuracy of 100%.

A panel may be selected such that when one or more regions or genes in the panel are removed, specificity is appreciably decreased. Removal of one region from the panel may result in a decrease in specificity of at least 5%, 10%, 15%, 20%, 25%, 30%, 35%, 40%, 45%, 50%, or more.

A panel may be selected such that the addition of one or more regions or genes to the panel does not appreciably increase the specificity of the panel, e.g., does not increase the specificity by more than 1%, 2%, 5%, 10%, 15%, or 20%.

A panel may be of a size such that when one or more regions or genes in the panel are removed, this appreciably decreases sensitivity, e.g., sensitivity is decreased by at least 5%, 10%, 15%, 20%, 25%, 30%, 35%, 40%, 45%, 50%, or more.

A panel may be selected such that the addition of one or more regions or genes to the panel does not appreciably increase the sensitivity of the panel, e.g., does not increase the sensitivity by more than 1%, 2%, 5%, 10%, 15%, or 20%.

A panel may be of a size such that when one or more regions or genes in the panel are removed, accuracy is appreciably decreased, e.g., accuracy is decreased by at least 5%, 10%, 15%, 20%, 25%, 30%, 35%, 40%, 45%, 50%, or more.

A panel may be selected such that the addition of one or more regions or genes to the panel does not appreciably increase the accuracy of the panel, e.g., does not increase the accuracy by more than 1%, 2%, 5%, 10%, 15%, or 20%.

A panel may be of a size such that when one or more regions or genes the panel are removed, positive predictive value is appreciably decreased, e.g., positive predictive value is decreased by at least 5%, 10%, 15%, 20%, 25%, 30%, 35%, 40%, 45%, 50%, or more.

A panel may be selected such that the addition of one or more regions or genes to the panel does not appreciably increase the positive predictive value of the panel, e.g., does not increase the positive predictive value by more than 1%, 2%, 5%, 10%, 15%, or 20%

A panel may be selected to be highly sensitive and detect low frequency genetic variants. For instance, a panel may be selected such that a genetic variant or biomarker present in a sample at a frequency as low as 0.01%, 0.05%, or 0.001% may be detected at a sensitivity of at least 50%, 55%, 60%, 65%, 70%, 75%, 80%, 85%, 90%, 95%, 96%, 97%, 98%, 99%, 99.5%, or 99.9%. Regions in a panel may be selected to detect a biomarker present at a frequency of 1% or less in a sample with a sensitivity of 70% or greater. A panel may be selected to detect a biomarker at a frequency in a sample as low as 0.1% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a biomarker at a frequency in a sample as low as 0.01% with a sensitivity of at least 50%, 55%, 60%, 65%, 70%, 75%, 80%, 85%, 90%, 95%, 96%, 97%, 98%, 99%, 99.5%, or 99.9%. A panel may be selected to detect a biomarker at a frequency in a sample as low as 0.001% with a sensitivity of at least 50%, 55%, 60%, 65%, 70%, 75%, 80%, 85%, 90%, 95%, 96%, 97%, 98%, 99%, 99.5%, or 99.9%.

A panel may be selected to be highly specific and detect low frequency genetic variants. For instance, a panel may be selected such that a genetic variant or biomarker present in a sample at a frequency as low as 0.01%, 0.05%, or 0.001% may be detected at a specificity of at least 50%, 55%, 60%, 65%, 70%, 75%, 80%, 85%, 90%, 95%, 96%, 97%, 98%, 99%, 99.5%, or 99.9%. Regions in a panel may be selected to detect a biomarker present at a frequency of 1% or less in a sample with a specificity of 70% or greater. A panel may be selected to detect a biomarker at a frequency in a sample as low as 0.1% with a specificity of at least 70%, 75%, 80%, 85%, 90%, 95%, 96%, 97%, 98%, 99%, 99.5%, or 99.9%. A panel may be selected to detect a biomarker at a frequency in a sample as low as 0.01% with a specificity of at least 70%, 75%, 80%, 85%, 90%, 95%, 96%, 97%, 98%, 99%, 99.5%, or 99.9%. A panel may be selected to detect a biomarker at a frequency in a sample as low as 0.001% with a specificity of at least 70%, 75%, 80%, 85%, 90%, 95%, 96%, 97%, 98%, 99%, 99.5%, or 99.9%.

A panel may be selected to be highly accurate and detect low frequency genetic variants. A panel may be selected such that a genetic variant or biomarker present in a sample at a frequency as low as 0.01%, 0.05%, or 0.001% may be detected at an accuracy of at least 70%, 75%, 80%, 85%, 90%, 95%, 96%, 97%, 98%, 99%, 99.5%, or 99.9%. Regions in a panel may be selected to detect a biomarker present at a frequency of 1% or less in a sample with an accuracy of 70% or greater. A panel may be selected to detect a biomarker at a frequency in a sample as low as 0.1% with an accuracy of at least 70%, 75%, 80%, 85%, 90%, 95%, 96%, 97%, 98%, 99%, 99.5%, or 99.9%. A panel may be selected to detect a biomarker at a frequency in a sample as low as 0.01% with an accuracy of at least 70%, 75%, 80%, 85%, 90%, 95%, 96%, 97%, 98%, 99%, 99.5%, or 99.9%. A panel may be selected to detect a biomarker at a frequency in a sample as low as 0.001% with an accuracy of at least 70%, 75%, 80%, 85%, 90%, 95%, 96%, 97%, 98%, 99%, 99.5%, or 99.9%.

A panel may be selected to be highly predictive and detect low frequency genetic variants. A panel may be selected such that a genetic variant or biomarker present in a sample at a frequency as low as 0.01%, 0.05%, or 0.001% may have a positive predictive value of at least 70%, 75%, 80%, 85%, 90%, 95%, 96%, 97%, 98%, 99%, 99.5%, or 99.9%.

The concentration of probes or baits used in the panel may be increased (2 to 6 ng/μL) to capture more nucleic acid molecule within a sample. The concentration of probes or baits used in the panel may be at least 2 ng/μL, 3 ng/μL, 4 ng/μL, 5 ng/μL, 6 ng/μL, or greater. The concentration of probes may be about 2 ng/μL to about 3 ng/μL, about 2 ng/μL to about 4 ng/μL, about 2 ng/μL to about 5 ng/μL, about 2 ng/μL to about 6 ng/μL. The concentration of probes or baits used in the panel may be 2 ng/μL or more to 6 ng/μL or less. In some instances this may allow for more molecules within a biological to be analyzed thereby enabling lower frequency alleles to be detected.

### Genetic Analysis

Genetic analysis includes detection of nucleotide sequence variants and copy number variations. Genetic variants can be determined by sequencing. The sequencing method can be massively parallel sequencing, that is, simultaneously (or in rapid succession) sequencing any of at least 100,000, 1 million, 10 million, 100 million, or 1 billion polynucleotide molecules. Sequencing methods may include, but are not limited to: high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next-generation sequencing, Single Molecule Sequencing by Synthesis (SMSS)(Helicos), massively-parallel sequencing, Clonal Single Molecule Array (Solexa), shotgun sequencing, Maxam-Gilbert or Sanger sequencing, primer walking, sequencing using PacBio, SOLiD, Ion Torrent, or Nanopore platforms and any other sequencing methods known in the art.

Sequencing can be made more efficient by performing sequence capture, that is, the enrichment of a sample for target sequences of interest, e.g., sequences including the KRAS and/or EGFR genes or portions of them containing sequence variant biomarkers. Sequence capture can be performed using immobilized probes that hybridize to the targets of interest.

Cell free DNA can include small amounts of tumor DNA mixed with germline DNA. Sequencing methods that increase sensitivity and specificity of detecting tumor DNA, and, in particular, genetic sequence variants and copy number variation, can be useful in the methods of this invention. Such methods are described in, for example, in WO 2014/039556. These methods not only can detect molecules with a sensitivity of up to or greater than 0.1%, but also can distinguish these signals from noise typical in current sequencing methods. Increases in sensitivity and specificity from blood-based samples of cfDNA can be achieved using various methods. One method includes high efficiency tagging of DNA molecules in the sample, e.g., tagging at least any of 50%, 75% or 90% of the polynucleotides in a sample. This increases the likelihood that a low-abundance target molecule in a sample will be tagged and subsequently sequenced, and significantly increases sensitivity of detection of target molecules.

Another method involves molecular tracking, which identifies sequence reads that have been redundantly generated from an original parent molecule, and assigns the most likely identity of a base at each locus or position in the parent molecule. This significantly increases specificity of detection by reducing noise generated by amplification and sequencing errors, which reduces frequency of false positives.

Methods of the present disclosure can be used to detect genetic variation in non-uniquely tagged initial starting genetic material (e.g., rare DNA) at a concentration that is less than 5%, 1%, 0.5%, 0.1%, 0.05%, or 0.01%, at a specificity of at least 99%, 99.9%, 99.99%, 99.999%, 99.9999%, or 99.99999%. Sequence reads of tagged polynucleotides can be subsequently tracked to generate consensus sequences for polynucleotides with an error rate of no more than 2%, 1%, 0.1%, or 0.01%.

Copy number variation determination can involve determining a quantitative measure of polynucleotides in a sample mapping to a genetic locus, such as the EGFR gene or KRAS gene. The quantitative measure can be a number. Once the total number of polynucleotides mapping to a locus is determined, this number can be used in standard methods of determining Copy Number Variation at the locus. A quantitative measure can be normalized against a standard. In one method, a quantitative measure at a test locus can be standardized against a quantitative measure of polynucleotides mapping to a control locus in the genome, such as gene of known copy number. In another method, the quantitative measure can be compared against the amount of nucleic acid in the original sample. For example, the quantitative measure can be compared against an expected measure for diploidy. In another method, the quantitative measure can be normalized against a measure from a control sample, and normalized measures at different loci can be compared. In another method, quantifying involves quantifying parent or original molecules in a sample mapping to a locus, rather than number of sequence reads. A copy number variation may be an amplification or a deletion or truncation of a gene. An amplification may be 3, 4, 5, 6, 7, 8, 9, 10, or 10 or more copies of a gene. A deletion or truncation may be 0 or 1 copies of a gene.

An example of a method for detecting copy number variation may include an array. The array may comprise a plurality of capture probes. The capture probes can be oligonucleotides that are bound to the surface of the array. The capture probes may hybridize to at least one of the genes as set forth in Table 1. The capture probes may bind to at least 1, 2, 3, 4, 5, 6, 7, 8, 9, 10, 11, or 12 genes as set forth in Table 1. DNA derived from the subject may be labeled (e.g., with a fluorophore) prior to hybidization for detection.

In other examples, a gene of interest may be amplified using primers that recognize the gene of interest. The primers may hybridize to a gene upstream and/or downstream of a particular region of interest (e.g., upstream of a mutation site). A detection probe may be hybridized to the amplification product. Detection probes may specifically hybridize to a wild-type sequence or to a mutated/variant sequence. Detection probes may be labeled with a detectable label (e.g., with a fluorophore). Detection of a wild-type or mutant sequence may be performed by detecting the detectable label (e.g., fluorescence imaging). In examples of copy number variation, a gene of interest may be compared with a reference gene. Differences in copy number between the gene of interest and the reference gene may indicate amplification or deletion/truncation of a gene. Examples of platforms suitable to perform the methods described herein include digital PCR platforms such as e.g., Fluidigm Digital Array.

### EXAMPLES

### Example 1—Performance Characteristics

It was observed that detection can occur when tumor fraction is ˜20%. If ˜20% TF requirement is measured, this performance in liquid is both sufficient and noteworthy. For example, some CDx deletion biomarker have 40% or greater TF requirement. Nevertheless, a, 20% tumor fraction could be regarded as a detection rate that is low when tested in a Tissue +ve cohort.

### Example 2—Identifying a Methylation Signature

For this aim, identifying a methylation signature that has lower TF requirement would provide beneficial. In some instance, one can indirectly, albeit effectively, genotype MTAP deletion. Without being bound by any particular theory, MTAP deletion causes major biochemical changes to the cell, which can be readout in distinct methylation pattern.

In an illustrative example, clustering samples by methylation markers show potential for methylation signature of MTAP status: MTAP deletion enriched, MTAP depleted, and intermediate clusters.

Additionally, it was observed that samples with MTAP loss (LoH or Homozgous deletions) trend toward distinct cluster from samples with intact MTAP. Here, a minimum >10% TF for all included samples was applied. Of further interest is to expand subset of MTAP-deleted samples, which may minimize batch-specific bias.

### Example 3—a Signature that Captures MTAP Deletion, Loss of Function

Of interest is determining a signature that captures MTAP LoF, which one of skill appreciates can characterize the far reaching effects well beyond MTAP deletion alone. For example, conventional measurement, if singularly limited to subjects afflicted MTAP copy number loss, may not capture the broadest possible patient population that could benefit from a therapy wherein MTAP LoF more widely present.

Herein, the Inventors have devised an approach for determination of methylation signature that broadens the population eligible for therapy beyond current genomic MTAP CN loss population. This is provided by the measurement of 20K+ different methylated regions (DMRs) in a holistic manner, which can capture MTAP pathway functional state and broaden patient population for therapies. More specifically, regions selected are based on MTAP comparing samples with known MTAP deletions to matched (cancer type and tumor fraction) samples that did not have MTAP deletions. Given the wide ranging effects of MTAP dysfunction, loss of function in MTAP will likely have an analogous methylation pattern to MTAP deletion within the methylation regions selected.

In one example, patients are pre-screened by tissue for MTAP CN loss=200 training+200 validation), as well as −200 MTAP CN loss −ve samples by tissue and liquid result from epigenomic methylation detection. Of interest is identifying those patient samples not tissue +ve for MTAP loss for signature validation and their outcomes.

Briefly, in a first step, MTAP CN loss samples by tissue screening further curated by epigenomic methylation detection provides higher resolution characterization of 9p21.3 locus and MTAP loss. Thereafter, one can develop a signature indicative of MTAP loss-of-function.

The aforementioned 20K+ differential methylation regions capture MTAP pathway functional state and broaden patient population for therapies. In addition, this may further include promoter methylation and other DMRs. In a subsequent step, one can train/test predictive score/model via −200 MTAP CN loss +ve as well as −200 MTAP CN loss −ve Build score/model from DMRs identified previously and for prediction of MTAP pathway functional state.

Finally, analytical and clinical validation in future study supports the use of predictive score as an element in a future trial to enroll a broader population. Other mechanisms of MTAP LoF have greater potential for wide uses of therapies. For example, loss of function of a transcription factor regulating the expression of the MTAP pathway (e.g., master regulator) or a mutation or epigenetic mark(histone/methylation) at an enhancer that regulates expression of multiple MTAP pathway genes (e.g., rearrangement of promoter-enhancer looping).

Other examples of MTAP LoF, include deletion, promoter methylation, posttranslational modifications that direct proteins for degradation by the proteasome, etc. In other words, the method can be used to detect MTAP deletion and MTAP LoF.

### Example 4—Predictive Methyl Signature for Therapies

Of interest is identifying signatures that identifies who will respond to therapeutic agents at baseline. These predictive methyl signature can be highly informative in treatment decision making, including the new class of protein arginine methyltransferase 5 (PRMT5) inhibitors that have shown promising preliminary clinical efficacy.

Detecting MTAP deletion is only one way of determining MTAP LoF, and does not capture other mechanisms/pathway changes. Methylation signature can capture the broader range of mechanisms that lead to MTAP LoF.

As an example, the Inventors have previously used this approach to identify predictive biomarkers for drug efficacy. For example, blinatumomab, a bispecific T-cell engager, has strong tumor activity in ALL, but efficacy in other heme malignancies has not been confirmed. Here, the Inventors established means to identify blinatumomab predictive biomarkers across various malignancies.

More specifically, analysis can be described as follows: ˜400 blinatumomab-treated patients with outcomes (200 training+200 validation), with clinical outcomes (PFS) for validation. Thereafter, a variety of assessments are applied including, molecular response classification, wherein one classifies responder/non-responder via tumor quantification from thousands of differentially methylated regions (DMRs) between baseline and early on-treatment timepoints. Optionally, one can confirm response with imaging from the study.

Thereafter, comparison of MR groups can identify effect size and mutual information of 20K+ normalized DMRs/genomic markers will serve to identify small subset of DMRs that have the highest predictive power in differentiating responders from non-responders.

Further, one can train and test predictive score models via ˜200 patients serves to build score/model from subset of DMRs that have the highest predictive power. Uses baseline sample data from earlier steps predicts patient response. Validation can be made by analysing whether predictive score can identify positive patient cohort with clinically meaningful superior response. Of use is clinical outcomes (ORR and PFS) in addition to MR and imaging. Thereafter, such model can be subject to analytical and clinical validation in future study for use of predictive score as an element in a future prospective studies, accompanied by focus via biomarker stratification.

### Example 5—Closed-Loop and Real World Evidence

Here, a chief aim is to better understand how MTAP LoF patients respond to current SOC treatments. Closed-loop capability consists of multiple analytical approaches, including validation of historical diagnostic results using RWE, optimization and development of new bioinformatic algorithms based on RWE insights and development and validation of new products via running historical samples on the new test and validating their analytical and clinical performance using RWE. This includes ascertaining biomarker status, and clinical diagnoses, as well as their outcomes summarized in variables such as real world time to next treatment (rwTTNT), and real world survival (rwOS).

### Example 6—Epigenotyping, Deletion Caller

The MTAP gene, located at chromosomal locus 9p21, is flanked by CDKN2A and miR-31 and the gene is frequently co-deleted, in many different tumors, with the CDKN2A and CDKN2B genes that encode the tumor suppressors p15, p16, p19 and with the genes for interferons alpha and beta that lie telomeric to miR-31, Selective MTAP deficiency, without co-deletion of the CDKN2 genes, has also been reported, due either to selective deletion of the MTAP locus or to methylation of the MTAP promoter.

Traditionally, three types of sequence data based signals are used for deletion detection, including discordant reads pairs, reads depth and split reads. Discordant read pairs are the reads pairs that are inconsistent with the reference genome. Read pairs that are mapped too far apart may be related to deletions. Read-depth approaches assume a random distribution in mapping depth and deletions show reduced read depth when compared to wild-type regions. Split reads are single reads that are mapped to the reference genome discontinuously as two or more segments. A potential breakpoint is used as the basis of a split sequence-read signature disturbs alignment of a read into multiple segments on the reference.

There are some limitations for those three signals. Discordant read pairs may give inexact positions of breakpoints. Split-read methods have are time consuming with high false positive and false negative rates. Read depths are not able to identify smaller events and much poorer at localizing breakpoints. In short, actual identification of the deletion site is largely ineffective given any of the aforementioned techniques.

Moreover, signal intensity may vary. A homozygous deletion, having only the deletion allele, can therefore be expected to have a stronger signal for split reads and discordant read pairs. A stronger signal makes a homozygous variant easier to detect than a heterozygous variant. Methods failing to detect homozygous variants are virtually guaranteed to detect heterozygous variants.

Here, the aforementioned obstacles are obviated by indirectly genotyping MTAP deletion since MTAP deletion is known to cause major biochemical changes to the cell. Instead of the limitations of sequence reads described above, the deletion is instead readout in distinct methylation pattern. One of skill readily appreciates such approach could be applied in other forms of genotyping, such as detecting fusions, indels, etc.

Insertion and deletion calling is highly dependent on double-stranded molecules and sometimes generates unstable results. Here, the current approach is more robust and aggregates information from both double stranded molecules and single stranded non-singleton molecules. Whereas dependence on tumor fraction for MTAP deletion calling may be high, using thousands/hundreds of hyper methylated regions may lower the TF requirement.

### Example 7—Synthetic Lethality

As described, some therapeutic interventions have been posited as related to MTAP deletion and dysfunction. This includes protein arginine methyltransferase 5 (PRMT5) inhibitors, which appear to be capable as functioning as a synthetic lethal target for MTAP-deleted (MTAP del) cancers. However, characterization of the purported synthetic lethal phenotype has been postulated, but the scope of this mechanisms has not been described.

Without being bound by any particular theory, selective inhibition of PRMT5 in the presence of MTA, which is elevated in MTAP del cancers, may effectuate antitumor activity and inhibition of PRMT5-dependent SDMA modification in MTAP del tumors. However, elucidating this mechanism may reveal minimal effects in WT, while also identifying possible other points of intervention or side effects. In addition, observing responses in patients with MTAP del may identify dose dependent response, if any, while also identifying efficacy in different cancers, such as adenocarcinoma, non-small cell lung cancer, and other malignant tumors may exhibit different amounts of responsiveness due to underlying dysfunction.

### Example 8 Expanding MTAP Diagnostic Concept

Described herein is the approach for methylation signature model development, including description of training performance and detection specificity characteristics. Demonstrated herein is the detection rate among development samples and overlap between genomic and methylation based calls, wherein the described methylation based detection scheme overlaps, but notably broadens the patient population detected compared to genomic only techniques.

Typically, it is understood that liquid biopsy can expand the testable population and increases enrollment efficiency but is associated with decreased sensitivity for MTAP deletion due to tumor shedding. However, as shown detection rate more than doubles by addition of methyl signature. Additionally, liquid signature is available for many advanced cancer patients in which tissue is not available. MTAP promoter hypermethylation is mutually exclusive to MTAP deletion and adds additional patients.

### Example 9—Region Behavior

Having established the efficacy of liquid methylation of interest is characterizing the liquid methylation regions differential between +ve and −ve and making comparison to what has been reported for TCGA methylation regions differential between +ve and −ve. For these determinations, biological characterization can be done using the following. Pathway analysis was done using the Database for Advanced Visual and Integrated Discovery (DAVID) 6.8 platform at david.ncifcrf.gov. P-values were corrected for multiple comparisons using the Benjamini-Hochberg method for lowering the false discovery rate. Benjamini-Hochberg calculation was performed in DAVID using the default EASE score (modified Fisher's exact P-value)<0.1. Benjamini Hochberg FDR adjusts for multiple comparisons using the rank of the p value, the total number of comparisons, and (in the DAVID platform) the EASE score (0.1) and a minimum requirement of 2 genes per term. Additional statistical tests are performed using Graphpad Prism (t tests, log rank test for Kaplan-Meier curves, ANOVAs) and Statgraphics Centurion (ANOVA, Kruskal Wallis, Mann Whitney U).

Applying the above, 46 promoter methylation regions were identified as candidate features. Identifying genes associated with the promoter methylation regions and running DAVID pathway analysis, the Inventor observed 8 enrichment clusters with high corrected p-value.

### Example 10—Enrichment Data of MTAP Deleted and MTAP Intact Samples

After identifying individual DMR regions of interested, of interest is identifying true enrichment between MTAP intact and MTAP deleted samples. Here, it is important to understand how other factors like tumor fraction, inherent noise in the regions etc affect the analysis. Further, the Biological relevance of the DMRs observed can be determined as follows.

Here, 62 in the initial 96 DMR regions without baseline noise and TF dependence are taken to be our final DMR regions which show enrichment between MTAP intact and MTAP null samples.

Thereafter, the Inventors identified the closest genes associated with the final DMR regions and run DAVID pathway analysis. Here the Inventors identified 7 enrichment clusters with high corrected p-value. Further works needs to be done to further fine tune this analysis.

### Example 11—Additional Epigenotyping Applications Using Deletion Caller

As shown herein, the Inventors were able to utilize the aforementioned methods and techniques in utilizing the epigenotyping deletion caller for detecting PTEN and RB1 deletion in a manner similar to MTAP.

It is of interest to identify those regions with strong cancer enriched prevalence that are positioned next to key genes known to be deleted in cancer. This systematic inclusion of genes commonly deleted in cancer important because epigenotyping approaches, including for deletion detection, is best supported by monitoring regions with strong cancer enriched prevalence that are positioned next to the gene of interest. Also, an important aspect is incorporating regions where hypermethylation is biologically increased when the tumor has the gene deletion, and/or include sample level tumor methylation tumor fraction as a covariate. This is to mainly to guard against false positives since the part A method above is always co-deletion of the flanking cancer enriched region e.g. reduction in hypermethylation, which could look similar in liquid to a sample with low tumor fraction.

A variety of genes related to the methods and techniques described herein, including biomarkers and genetic targets that may be included in a sequencing panel as described.

### Example 12—Additional Epigenotyping Applications (SNVs, Indels, Etc.)

Further shown herein, the Inventors were able to utilize the aforementioned methods and techniques in utilizing the epigenotyping approach for detecting SNVs and indels, including application for example, in detecting EGFR activating SNVs and small indels (L858R/ex19 deletion and a few other EGFR activating SNVs).

In view of the foregoing, a multi-part analytical approach includes the following framework for detecting deletions, fusions, amplifications:
(1) Sensitive Region-level methylation detection, Region-level methylation LoD ˜1% MAF. Much more sensitive than deletion or amplification LoD;
(2) Prevalent, cancer-specific methylation Some regions are frequently methylated in cancer. E.g. >15% of all cancer samples. Unlike coverage profile, cancer-specific methylation only has signal from tumor.
(3) Deletion or amplification impacts Mb of DNA. Even focal deletions or amplifications co-delete Mb surrounding DNA, including methylated loci.
(4) Mutual exclusivity. Cancer-specific region methylation is present when region is not deleted in tumor and NEVER observed when region is deleted in the tumor;
(5) Multiple beacons. A single cancer-specific methylated region is insufficient, but multiple such regions act together as beacon for deletion/amplification;
(6) Build model to integrate beacons to detect loss or gain of tumor specific DNA at low tumor fraction.

1. A method, comprising:
generating a methylation profile for at least one nucleic acid sequence obtained from a human subject; and
selecting a treatment suitable for the human subject based on the methylation profile.

2. The method of claim 1, wherein the methylation profile comprises at least one differentially methylated region (DMR).

3. The method of claim 1, wherein the at least one DMR is determined based on a comparison to a threshold determined from one or more healthy subjects.

4. The method of claim 1, wherein the methylation profile characterizes a gain or loss of function for at least one or more genes.

5. (canceled)

6. The method of claim 1, wherein the methylation profile characterizes a genomic mutation.

7. The method of claim 1, wherein the methylation profile characterizes a gene target.

8. The method of claim 7, wherein the methylation profile characterizes gene dysfunction.

9. The method of claim 7, wherein the characterization is of a gene target capable of conferring synthetic lethality.

10. The method of claim 7, wherein the characterization is based on hypermethylation status.

11. The method of claim 4, wherein the one or more genes comprises CDKN2A.

12. The method claim 4, wherein the one or more genes comprises a metabolic enzyme.

13. The method of claim 12, wherein the metabolic enzyme comprises Methylthioadenosine Phosphorylase (MTAP).

14. The method of claim 1, wherein the methylation profile is detected using a methyl binding domain (MBD) partitioning assay.

15. The method of claim 14, wherein the MBD partitioning assay comprises combining a plurality of nucleic acid molecules derived from the human subject with a solution including an amount of methyl binding domain (MBD) proteins to produce a nucleic acid-MBD protein solution; and performing a plurality of washes of the nucleic acid-MBD protein solution with a salt solution to produce a number of nucleic acid fractions, individual nucleic acid fractions having a threshold number of methylated cytosines in regions of the plurality of nucleic acids having at least the threshold cytosine-guanine content.

16. The method of claim 1, wherein the treatment comprises an PRMT5-MTA5 inhibitor and/or immunotherapy.

17. The method of claim 6, 7, or 8, wherein the genomic mutation and/or gene dysfunction modulates PRMT5-MTA complex and/or MTA accumulation.

18. The method of claim 14, wherein the detection comprises a method selected from the group consisting of Next Generation Sequencing (NGS), Third-Generation Sequencing (TGS), Sanger Sequencing, Microarray Analysis, PCR, Fluorescence In Situ Hybridization (FISH), Real-Time PCR (RT-PCR), Digital PCR (dPCR), Droplet Digital PCR (ddPCR), Targeted Sequencing, Nanopore Sequencing, Short-Read Sequencing, Long-Read Sequencing Technologies (e.g., PacBio and Nanopore).

19. The method of claim 1, wherein the subject is afflicted with a cancer selected form the group consisting of: breast cancer, lung cancer, prostate cancer, colorectal cancer, skin cancer, ovarian cancer, pancreatic cancer, leukemia, lymphoma, liver cancer, cervical cancer, brain cancer, stomach cancer, kidney cancer, bladder cancer, thyroid cancer, esophageal cancer, melanoma, testicular cancer, epithelioid sarcoma, relapsed or refractory follicular lymphoma, non-Hodgkin lymphomas (NHL), relapsed or refractory adult T-cell leukaemia/lymphoma (R/R ATL), follicular lymphoma, castration-resistant prostate cancer (CRPC), small cell lung cancer, and sarcoma.

20. The method of claim 1, wherein the sample is selected from the group consisting of blood, plasma, cell free DNA(cfDNA), cell free RNA(cfRNA), saliva, urine, cerebrospinal fluid, synovial fluid, amniotic fluid, lymph, semen, vaginal fluid, tears, breast milk, mucus, sweat, pericardial fluid, pleural fluid, peritoneal fluid, bile, and interstitial fluid.

21. The method of claim 1, comprising selecting a treatment suitable for the human subject comprises use of a database.

22. The method of claim 21, wherein the database comprises a plurality of nucleic acid sequence information, including methylation status from a plurality of subjects.

23. The method of claim 1, wherein selecting a treatment comprises identifying from a plurality of subjects with matching genetic, epigenetic information, prior treatment of the plurality of subjects with matching genetic information.

24. A method, comprising:
generating a methylation profile using a methyl binding domain (MBD) partitioning assay for at least one nucleic acid sequence obtained from a human subject;
selecting a treatment suitable for the human subject based on the methylation profile using a database, wherein the database comprises a plurality of nucleic acid sequence information, and methylation status from a plurality of subjects and identifying from the plurality of subjects with matching genetic and epigenetic information, prior treatment of the plurality of subjects with matching genetic information.
